(19) United States
(12) Patent Application Publication
(10) Pub. No.: US 2002/0068302 A1
(43) Pub. Date: Jun. 6, 2002

(54) Clasp-4 transmembrane protein

(76) Inventors: Peter S. Lu, Mountain View, CA (US); Jonathan D. Garman, San Jose, CA (US); Albert F. Candia III, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/736,969

(22) Filed: May 7, 2001

Related U.S. Application Data
(60) Provisional application No. 60/240,508, filed on Oct. 13, 2000.

(51) Int. Cl. G01N 33/53; C07H 21/02; C07H 21/04; C07K 1/00; C07K 14/00; C07K 17/00

(52) U.S. Cl. 435/7.1; 530/350; 536/23.1

(57) ABSTRACT

The present invention relates to a cell surface molecule, designated cadherin-like asymmetry protein-4 (“CLASP-4”). In particular, it relates to CLASP-4 polynucleotides, polypeptides, fusion proteins, and antibodies. The invention also relates to methods of modulating an immune response by interfering with CLASP-4 function.

### CROSS-REFERENCE TO RELATED APPLICATIONS

This application claims priority to U.S. application Ser. Nos. 60/240,508, 60/240,503, 60/240,539, 60/240,543 (all filed Oct. 13, 2000); 09/547,276, 60/196,267, 60/196,527, 60/196,528, 60/196,460 (all filed Apr. 11, 2000); 60/182,296 (filed Feb. 14, 2000), 60/176,195 (filed Jan. 14, 2000), 60/170,453 (filed Dec. 13, 1999), 60/162,498 (filed Oct. 29, 1999), 60/160,860 (filed Oct. 21, 1999).

### FIELD OF THE INVENTION

The present invention relates to molecules expressed in cells of the immune system. In particular, the invention relates to a transmembrane protein that contains certain classical cadherin characteristics.

### BACKGROUND OF THE INVENTION

The generation of an immune response against an antigen is carried out by a number of distinct immune cell types that work in concert within the context of a particular antigen. The helper T cell (T_{H}) plays a pivotal role to coordinate two types of antigen-specific immune response; i.e., cellular and humoral immune response. Recognition of antigen by T cells requires the formation of a specialized junction between the T cell and the antigen-presenting cell (APC) called the “immulogical synapse” (Dustin, et al., 1998, Cell 94: 667-677). The immune synapse orchestrates recruitment and exclusion of specific proteins from the contact area by an unknown mechanism and is thought to be initiated by T-cell antigen receptor (TCR) recognition of peptides bound to MHC molecules (antigen) (Monk, et al. 1998, Nature 395: 82). However, the low affinity of the TCR for antigen as well as limited number of ligands makes it unlikely that TCR: antigen interaction alone is sufficient to drive the formation of the immunological synapse (Matsui et al., 1994, Proc. Natl. Acad. Sci. U.S.A. 91: 12861-12866).

Costimulatory molecules such as CD4, ICAM-1, LFA-1, CD28, CD2 have been proposed to stabilize the cell-cell contact (Dustin, et al., 1999, Science 283: 649). However, since these molecules are recruited to the synapse after activation they cannot account for the high specificity and avidity during the early phases of T-cell antigen recognition. Recent work demonstrated that a portion of the T cell surface at the leading edge is specialized to mediate the early phases of synapse formation (Negulescu, et al., 1996, Immunity 4: 421-430). Such a specialization must be a pre-formed structure containing cell surface adhesion proteins (ectodomains) to augment TCR engagement and corresponding cytoplasmic portions (endodomains) to transduce signals and bind cytoskeleton to maintain structural/functional polarity.

The ectodomain of the pre-formed synapse or “immune gateway” was recently discovered and is created in part by CLASP-1 (U.S. Ser. No. 09/411,328, filed Oct. 1, 1999; PCT/US99/22996). In addition to cadherin motifs, CLASP-1 also contains a CRK-SH3 binding domain, tyrosine phosphorylation sites, and coiled/coil domains suggesting direct interaction with cytoskeleton and regulation by adaptor molecules such as CRK. The CLASP-1 transcript is present in lymphoid organs and neural tissue, and the protein is expressed by T and B lymphocytes and macrophages in the MOMA-1 subregion of the marginal zone of the spleen, an area known to be important in T: B cell interaction. CLASP-1 staining of individual T and B cells exhibits a preactivation structural polarity, being organized as a “ball” or “cap” structure in B cells, and forming a “ring”, “ball” or “cap” structure in T cells. The placement of these structures is adjacent to the microtubule-organizing center (“MTOC”). CLASP-1 antibody staining indicates that CLASP- 1 is at the interface of T-B cell conjugates that are fully committed to differentiation. Antibodies to the extracellular domain of CLASP-1 also block T-B cell conjugate formation and T cell activation.

### SUMMARY OF THE INVENTION

The present invention relates to a cell surface molecule, a member of a new multigene family designated cadherin-like asymmetry protein(s) (“CLASP(s)”). In particular, it relates to a polynucleotide comprising a coding sequence for CLASP-4, a polynucleotide that selectively hybridizes to the complement of a CLASP-4 coding sequence, expression vectors containing such polynucleotides, genetically-engineered host cells containing such polynucleotides, CLASP-4 polypeptides, CLASP-4 fusion proteins, therapeutic compositions, CLASP-4 domain mutants, antibodies specific for CLASP-4 polypeptides, methods for detecting the expression of CLASP-4, and methods of inhibiting an immune response by interfering with CLASP-4 function. A wide variety of uses are encompassed by the invention, including but not limited to, treatment of autoimmune diseases and hypersensitivities, prevention of transplantation rejection responses, and augmentation of immune responsiveness in immunodeficiency states.

In one aspect, the invention provides an isolated CLASP-4 polynucleotide that is: (a) a polynucleotide that has the sequence of SEQ ID NO: 1(b) a polynucleotide that hybridizes under stringent hybridization conditions to (a) and encodes a polypeptide having the sequence of SEQ ID NO:2 or an allelic variant or homologue of a polypeptide having the sequence of SEQ ID NO:2; or (c) a polynucleotide that hybridizes under stringent hybridization conditions to (a) and encodes a polypeptide with at least 25 contiguous residues of the polypeptide of SEQ ID NO:2; or (d) a polynucleotide that hybridizes under stringent hybridization conditions to (a) and has at least 12 contiguous bases identical to or exactly complementary to SEQ ID NO: 1.

In one aspect, the invention provides a CLASP-4 polynucleotide that encodes a polypeptide having the full-length sequence of SEQ ID NO:2. In another aspect, the invention provides a CLASP-4 polynucleotide having the full-length sequence of SEQ ID NO: 1 of fragment thereof. In another aspect of the invention, the cDNA sequence (or protein coding sequence) is encoded by the inserts of ATCC Deposit Nos. PTA-1566, PTA-1572, PTA-2608 or PTA-2610.

In another aspect, the invention further provides an isolated CLASP-4 polynucleotide comprising a nucleotide sequence that has at least 90% percent identity to SEQ ID NO: 1 as calculated using FASTA wherein said sequences are aligned so that highest order match between said sequences is obtained.

The invention further provides an isolated polypeptide comprising a nucleotide sequence that has at least 90% sequence identity to SEQ ID NO:2 and is immunologically crossreactive with SEQ ID NO:2 or shares a biological function with native CLASP-4.

The invention also provides vectors, such as expression vectors, comprising a polynucleotide sequence of the invention In other embodiments, the invention provides host cells or progeny of the host cells comprising a vector of the invention. In certain embodiments, the host cell is a eukaryote. In other embodiments, the expression vector comprises a CLASP-4 polynucleotide in which the nucleotide sequence of the polynucleotide is operatively linked with a regulatory sequence that controls expression of the polynucleotide in a host cell. In certain embodiments, the invention provides a host cell comprising a CLASP-4 polynucleotide, wherein the nucleotide sequence of the polynucleotide is operatively linked with a regulatory sequence that controls expression of the polynucleotide in a host cell, or progeny of the cell.

In another aspect, the invention further provides a CLASP-4 polynucleotide that is an antisense polynucleotide. In a preferred embodiment, the antisense polynucleotide is less than about 200 bases in length. In other embodiments, the invention provides an antisense oligonucleotide complementary to a messenger RNA comprising SEQ ID NO: 1 and encoding CLASP-4, wherein the oligonucleotide inhibits the expression of CLASP-4.

In another aspect, the invention provides an isolated DNA that encodes a CLASP-4 protein as shown in SEQ ID NO:2. In certain embodiments, the CLASP-4 polynucleotide is RNA.

The invention provides a method for producing a polypeptide comprising: (a) culturing the host cell containing a CLASP-4 polynucleotide under conditions such that the polypeptide is expressed; and (b) recovering the polypeptide from the cultured host cell or its cultured medium.

The invention further provides an isolated CLASP-4 polypeptide encoded by a CLASP-4 polynucleotide. In some embodiments, the CLASP-4 polypeptide has the amino acid sequence of SEQ ID NO:2, or a fragment thereof. In some embodiments, the isolated CLASP-4 polypeptide is cell-membrane associated. In other embodiments, the isolated CLASP-4 polypeptide is soluble. In other embodiments, the soluble CLASP-4 polypeptide is fused with a heterologous polypeptide.

The invention further provides an isolated CLASP-4 protein having the sequence as shown in SEQ ID NO:2. In some embodiments, the invention provides a CLASP-4 protein comprising the sequence as shown in SEQ. ID. NO:1 and variants thereof that are at least 95% identical to SEQ ID. NO:2 and specifically binds a cytoskeletal protein. In certain embodiments the cytoskeletal protein is spectrin.

The invention further provides an isolated antibody that specifically binds to a polypeptide having the amino acid sequence as shown in SEQ ID NO:2, or a binding fragment thereof. In some embodiments the antibody is monoclonal. In other embodiments, the invention provides a hybridoma capable of secreting the antibody.

The invention further provides a method of identifying a compound or agent that binds a CLASP-4 polypeptide comprising: i) contacting a CLASP-4 polypeptide with the compound or agent under conditions which allow binding of the compound to the CLASP-4 polypeptide to form a complex and ii) detecting the presence of the complex.

The invention further provides a method of detecting a CLASP-4 polypeptide in a sample, comprising: (a) contacting the sample with a CLASP-4 antibody or binding fragment and (b) determining whether a complex has been formed between the antibody and with CLASP-4 polypeptide.

The invention further provides a method of detecting a CLASP-4 polypeptide in a sample, comprising: (a) contacting the sample with a CLASP-4 polynucleotide or a polynucleotide that comprises a sequence of at least 12 nucleotides and is complementary to a contiguous sequence of the CLASP-4 polynucleotide and (b) determining whether a hybridization complex has been formed.

The invention further provides a method of detecting a CLASP-4 nucleotide in a sample, comprising: (a) using a polynucleotide that comprises a sequence of at least 12 nucleotides and is complementary to a contiguous sequence of a CLASP-4 polynucleotide in an amplification process; and (b) determining whether a specific amplification product has been formed.

The invention further provides pharmaceutical compositions comprising a CLASP-4 polynucleotide, a CLASP-4 polypeptide, or a CLASP-4 antibody and a pharmaceutically acceptable carrier.

In one aspect, the invention provides a method of inhibiting an immune response in a cell comprising: (a) interfering with the expression of a CLASP-4 gene in the cell; (b) interfering with the ability of a CLASP-4 protein to mediate cell-cell interaction (e.g., interfering with a heterotypic and/or homotypic interaction) between CLASP-4 and an extracellular protein; (c) interfering with the ability of a CLASP-4 protein to bind to another protein. In some such methods, the cell is a T cell or a B cell. Some such methods comprise contacting the cell with an effective amount of a polypeptide which comprises the amino acid sequence of SEQ ID NO:2 or a fragment thereof.

In another aspect, the invention provides a method of inhibiting an immune response in a subject, comprising administering to the subject a therapeutically effective amount of an antibody which specifically binds a polypeptide having the sequence of SEQ ID NO:2.

In another aspect, the invention provides a method of preventing or treating a CLASP-4-mediated disease comprising administering to a subject in need thereof a therapeutically effective amount of a CLASP-4 pharmaceutical composition. In some such methods, the CLASP-4-mediated disease is an autoimmune disease.

The invention further provides a method of treating an autoimmune disease in a subject caused or exacerbated by increased activity of T_{H}1 cells consisting of administering a therapeutically effective amount of a CLASP-4 pharmaceutical composition to the subject.

### BRIEF DESCRIPTION OF THE DRAWINGS

FIG. 1. Preliminary CLASP-4 cDNA sequence. Notable protein motifs are labeled above the nucleotide sequence.

FIG. 2. Expression of CLASP-4 in human cell lines and human tissues as determined by Northern hybridization. A CLASP-4-specific DNA fragment (HC4.3) was generated by PCR from a CLASP-4 cDNA clone, using primers129333F2 and 12933R3 (spanning nucleotides 2987-3660 of the cDNA sequence presented in FIG. 1). The fragment was labeled by incorporation of radioactive 32P dCTP. A. Expression in human tissues. The labeled DNA fragment was used as a probe on a human Multiple Tissue Northern (Clonetech MTN Blot, #7780-1). A single band is clearly detected migrating at approximately 7.5 kb only with strong expression only in peirpheral blood lymphocytes (PBL). Slight expression is detected in lung, placenta, small intestine, liver, kidney, spleen, thymus, and heart. B. Expression in hematopoietic cell lines. A Northern with RNA from multiple cells lines was hybridized with the same hCLASP-4 probe. A similarly migrating band is detected in Jurkat (T-cell derived), MV4- 11 (myelomonocytic), 9D10 (B-cell derived) and 293 (human kidney derived) cell lines.

FIG. 3. A. Amino acid sequence of human and rat CLASP proteins. Sequences were aligned using ClustalW. One letter amino acid abbreviation used. Protein motifs are found within the labeled boxes. A “-” indicates gaps that are placed to acquire a best overall alignment. Other abbreviations: “HC2A” Human CLASP-2 sequence, “KIAA” KIAA1058 sequence (Genbank Accession No. AB028981), “rat” TRG gene (Genbank Accession No. X68101), “HC4” Human CLASP-4 sequence, “HCl” Human CLASP-1 sequence, “HC3” Human CLASP-3 sequence, “HC5” Human CLASP-5 sequence. B. Alignment of DOCK motifs found within the human CLASPs and compared to canonical DOCK motifs. Consensus amino acids found within all DOCK motifs are also indicated.

FIG. 4. Southern hybridization analysis of CLASP-4. Genomic DNA prepared by from HeLa cells (ATCC # CCL-17) or a BAC DNA clone was digested with EcoRI or HinDIII (genomic DNA), or EcoRI or Pst I (BAC DNA) and eletrophoresed and transferred to nylon membrane by standard methods (Sambrook, Fritsch and Maniatis, 1989). For a probe, a CLASP-4-specific DNA fragment (HC4.1) was generated by PCR from a CLASP-4 cDNA, using primers HC4S5′ and HC4AS10. The fragment was labeled by incorporation of radioactive 32P dCTP. Probe HC4.1 is 606 bp long (spanning nucleotides 353-958 of the cDNA sequence shown in FIG. 1 and nucleotides 2319-2924 of the cDNA sequence shown in FIG. 5) and it recognizes four fragments (approximately sized at 1.3 kb, 2.8 kb, 4.1 kb and 5.5 kb) on HindIII digested DNA and three fragments (approximately sized at 1.6 kb, 11 kb and 13 kb) on EcoRI digested DNA.

FIG. 5. A. Full length cDNA sequence (SEQ ID NO:1) and predicted amino acid translation (SEQ ID NO:2) of the human CLASP-4 gene. Predicted initiator methionine starts at nucleotide +1. In-frame stop codons upstream of the initiator methionine support the fact that this sequence represents the full length coding region of CLASP-4. Ten independent 1st exons (indicated as 1A through 1J) were discovered and all splice into the second exon starting at nucleotide −94. These different first exons were discovered as RT-PCR products (“P2”), 5′ RACE products (“GR”) or as expressed sequence tags (“EST”) from Genbank. Note that some isoforms do not encode an in-frame stop codon and could encode a longer protein. The methionine encoded at nucleotide 2380 in FIG. 5 is equivalent to the first methionine predicted to be encoded at nucleotide 414 as shown in FIG. 1 above. The sequence presented in FIG. 1 corresponds to nucleotides 2380 to 6360 of FIG. 5. Numerous nucleotide differences exist between the regions in FIG. 1 and FIG. 5 around these methionines due to polymorphisms and the presence of sequences from a pseudogene encoded on chromosome 14. B) Differences between the human CLASP-4 cDNA isoforms. In addition to the differential first exon usage indicated in A), sequencing multiple, independent cDNA products revealed multiple nucleotide differences and a 5 base pair deletion, which may indicate allelic variations between CLASP-4 cDNA isoforms. Additionally, differential exon usage through alternative splicing events was discovered. In this particular case, the use of the exon in B leads to a premature stop codon that could generate a soluble form of CLASP-4. C) Schematic of human CLASP-4 cDNAs. The top line represents nucleotide numbering found in FIG. 5A. Line (i) represents CLASP-4 cDNA sequence as shown in FIG. 1; line (ii) represents the full length CLASP-4 isoforms, where there are ten CLASP-4 full length cDNA isoforms (A+Z, B+Z, C+Z, and so on). Each of the isoforms uses a first exon (A, B, C, and so on) that splices into the rest of the cDNA from exon 2 onwards represented by Z (see FIG. 5A). Line (iii) represents the additional 5′ sequence and overlap between nucleotides 1966 to 2737 shown in FIG. 5A and nucleotides 1-772 of FIG. 1.

FIG. 6. Sequence of human CLASP-4 exons and introns. A. Sequence of human CLASP-4 exons and intron borders. A contigous genomic sequence from the Human Genome Project (GENBANK entry gi5836219) was aligned using the human CLASP-4 cDNA as a template and Sequencher sequence analysis software. Due to the incompleteness of the Human Genome Project, only partial genomic sequence from human CLASP-4 was obtained. The ten independent 1st exons are presented in order (5′ to 3′) as they appear in genomic DNA. Exons 2-16 representing approximately the 5′ 30% of the human CLASP-4 cDNA sequence are presented in predicted 5′ to 3′ order. Exon sequences are underlined and are flanked by intron sequence. This exon/intron map could only have been produced having the isolated human CLASP-4 cDNA. Nucleotide numbers for each exon and flanking intron sequences are indicated and represent the reverse complement of the annotation found in Genbank entry gi5836219. B. Genomic nucleotide sequence upstream of the various CLASP-4 5′ cDNA termini, which represents the putative promoter regions for human CLASP-4. The 5′ termini of the different 1st exons of the CLASP-4 cDNA are underlined. Some potential transcription factor binding sites have been identified and marked corresponding to the key at the bottom of the figure.

FIG. 7. Amino acid alignment and comparison between the human (h) CLASP family members. Amino acid sequences were aligned using ClustalW. The alignment is presented in order of their greatest pairwise similarity scores. Single letter amino acid abbreviations are used. Astericks indicate complete identity, while colons and periods indicate sequence similarity among CLASP family members. Dashes indicate gaps inserted in the amino acid sequence to facilitate alignment. Labelled boxes are domains with similarity to known protein motifs; unlabelled boxes represent regions of similarity between all CLASPs and may represent CLASP-specific domains.

FIG. 8. Expression of CLASP-4 upon T-cell activation as assayed by Northern analysis. Jurkat cells were activated using PMA, Ionomycin, and anti-CD28 antibody. RNA was prepared from cell culture aliquots at 0, 1, 2, 4, 8, 14 hours post activation and Northern analysis was performed (A). Hybridization signals obtained with a CLASP-4 specific probe or the beta actin-specific probe, respectively, were quantified using a phosphor imager system. Relative signal intensities, including signals obtained from hybridization with CLASP-1 specific probe (refering to total signal of the specific probe used), are shown in the bar graph (B). The ethidium staining of the Northern gel (A) demonstrates even RNA loading.

### DETAILED DESCRIPTION OF THE INVENTION

Definitions

Except when noted, the terms “patient” or “subject” are used interchangeably and refer to mammals such as human patients and non-human primates, as well as experimental animals such as rabbits, rats, and mice, and other animals.

The term “biological sample” as used herein is a sample of biological tissue, fluid, or cells that contains hCLASP-4 or nucleic acid encoding hCLASP-4 protein. Such samples include, but are not limited to, tissue isolated from humans. Biological samples may also include sections of tissues such as frozen sections taken for histologic purposes. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans.

The term “treating” includes the administration of the compounds or agents of the present invention to prevent or delay the onset of the symptoms, complications, or biochemical indicia of a disease, alleviating the symptoms or arresting or inhibiting further development of the disease, condition, or disorder (e.g., autoimmune disease). Treatment may be prophylactic (to prevent or delay the onset of the disease, or to prevent the manifestation of clinical or subclinical symptoms thereof) or therapeutic suppression or alleviation of symptoms after the manifestation of the disease.

The term “lymphocyte” as used herein has the normal meaning in the art, and refers to any of the mononuclear, nonphagocytic leukocytes, found in the blood, lymph, and lymphoid tissues, i.e., B and T lymphocytes.

The terms “isolated,” or “purified,” refer to material that is substantially free from components that normally accompany it as found in its native state (e.g., recombinantly produced or purified away from other cell components with which it is naturally associated). Purity and homogeneity are typically determined using analytical chemistry techniques such as polyacrylamide gel electrophoresis or high performance liquid chromatography. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably”and refer to refers to DNA, RNA and nucleic acid polymers containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The amino acids may be natural amino acids, or include an artificial chemical mimetic of a corresponding naturally occurring amino acid.

As used herein a “nucleic acid probe” is defined as a nucleic acid capable of specifically binding to a target nucleic acid of complementary sequence (e.g., through complementary base pairing). As used herein, a probe may include natural (i.e., A, G, C, or T) or modified bases (7-deazaguanosine, inosine, and the like). In addition, the bases in a probe may be joined by a linkage other than a phosphodiester bond, so long as it does not interfere with hybridization (e.g., probes may be peptide nucleic acids). The probes can be directly labeled as with isotopes, chromophores, lumiphores, chromogens, or indirectly labeled such as with biotin to which a streptavidin complex may later bind.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or, in the case of cells, to progeny of a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “sequence identity” refers to a measure of similarity between amino acid or nucleotide sequences, and can be measured using methods known in the art, such as those described below:

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region (see, e.g., SEQ ID NO:1 ), when compared and aligned for maximum correspondence over a comparison window, or designated region as measured using one of the following sequence comparison algorithms or by manual alignment and visual inspection.

The phrase “substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

The phrase “sequence similarity” in the context of two nucleic acids or polypeptides, refers to two or more sequences that are identitical or in the case of amino acids, have homologous amino acid substitutions at either 50%, often at least 60%, often at least 70%, preferably at least 80%, most preferably at least 90% or at least 95% of the indicated positions.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to CLASP-4 nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2: 482), by the homology alignment algorithm of Needleman & Wunsch, 1970, J. Mol. Biol. 48: 443, by the search for similarity method of Pearson & Lipman, 1988, Proc. Natl. Acad. Sci. U.S.A. 85: 2444, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., 1987 (1999 Suppl.), Current Protocols in Molecular Biology, Greene Publishing Associates and Wiley Interscience, N.Y.)

A preferred example of an algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty =−2; and width=16.

Another preferred example of algorithm that is suitable for determining percent sequence identity and sequence similarity are the BLAST and BLAST 2.0 algorithms, which are described in Altschul et al., 1977, Nuc. Acids Res. 25: 3389-3402 and Altschul et al., 1990, J. Mol. Biol. 215: 403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http: //www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. U.S.A. 89: 10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Natl. Acad. Sci. U.S.A. 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35: 351-360. The method used is similar to the method described by Higgins & Sharp, 1989, CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12: 387-395.

Another preferred example of an algorithm that is suitable for multiple DNA and amino acid sequence alignments is the CLUSTALW program (Thompson, J. D. et al., 1994, Nucl. Acids. Res. 22: 4673-4680). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, 1992, Proc. Natl. Acad. Sci. U.S.A. 89: 10915-10919).

A “label” is a composition detectable by spectroscopic, photochemical, biochemical, immunochemical, or chemical means. For example, useful labels include 32P, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO:1 can be made detectable, e.g., by incorporating a radiolabel into the peptide, and used to detect antibodies specifically reactive with the peptide).

The term “sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

The phrase “specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions may require a binding moiety that is selected for its specificity for a particular target antigen. A variety of assay formats may be used to select ligands that are specifically reactive with a particular protein. For example, solid-phase ELISA immunoassays, immunoprecipitation, Biacore and Western blot are used to identify peptides that specifically react with PDZ domain-containing proteins. Typically a specific or selective reaction will be at least twice background signal or noise and more typically more than 10 times background. Specific binding between a monovalent peptide and a PDZ-containing protein means a binding affinity of at least 10⁴ M⁻¹, and preferably 10⁵ or 10⁶ M⁻¹.

The phrase “homotypic interaction” refers to the binding of a given protein to another molecule of the same protein (e.g., the binding of hCLASP-4 to hCLASP-4). The phrase “heterotypic interaction” refers to the binding of a given protein to a different protein or other molecule (e.g., a transcription factor to DNA).

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

The terms “B lymphocyte response” and “B lymphocyte activity” are used interchangeably to refer to the component of immune response carried out by B lymphocytes (i.e. the proliferation and maturation of B lymphocytes, the binding of antigen to cell surface immunogobulin, the internalization of antigen and presentation of that antigen via MHC molecules to T lymphocytes, and the synthesis and secretion of antibodies).

The terms “T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

The term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Components of an immune response may be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen A; et al., 1995, Immunity 2(4): 373-80), (3) antigen presenting cells can be incubated with whole protein antigen and the presentation of that antigen on MHC detected by either T lymphocyte activation assays or biophysical methods (Harding et al., 1989, Proc. Natl. Acad. Sci., 86: 4230-4), (4) mast cells can be incubated with reagents that cross-link their Fc-epsilon receptors and histamine release measured by enzyme immunoassay (Siraganian, et al., 1983, TIPS 4: 432-437).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., 1988, Blood 72: 1310-5); (3) the proliferation of peripheral blood mononuclear cells in response to mitogens or mixed lymphocyte reaction can be measured using 3H-thymidine; (4) the phagocitic capacity of granulocytes, macrophages, and other phagocytes in PBMCs can be measured by placing PMBCs in wells together with labeled particles (Peters et al., 1988); and (5) the differentation of immune system cells can be measured by labeling PBMCs with antibodies to CD molecules such as CD4 and CD8 and measuring the fraction of the PBMCs expressing these markers.

As used herein, the phrase “signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an immune response described above.

A signal transduction pathway refers to the biochemical relationship between a variety of signal transduction molecules that play a role in the transmission of a signal from one portion of a cell to another portion of a cell. Signal transduction molecules of the present invention include, for example, extracellular and intracellular domains of CLASP-4. As used herein, the phrase “cell surface receptor” includes molecules and complexes of molecules capable of receiving a signal and the transmission of such a signal across the plasma membrane of a cell. An example of a “cell surface receptor” of the present invention is the T cell receptor (TCR). As used herein, the phrase “intracellular signal transduction molecule” includes those molecules or complexes of molecules involved in transmitting a signal from the plasma membrane of a cell through the cytoplasm of the cell, and in some instances, into the cell's nucleus. In the present invention, CLASP-4 can be referred to as an “intracellular signal transduction molecule”, but can also be referred to as a “signal transduction molecule”.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; the conformational change of a molecule; calcium release; inositol phosphate production; proteolytic cleavage; cyclic nucleotide production and diacylglyceride production. Typically, signal transduction occurs through phosphorylating a signal transduction molecule. According to the present invention, a CLASP-4 signal transduction pathway refers generally to a pathway in which CLASP-4 protein regulates a pathway that includes engaged-receptors, PKC-substrates, G proteins, and other molecules.

Introduction

The present invention relates to a novel transmembrane protein, CLASP-4, a new member of the CLASP family that contains an endodomain that displays the appropriate properties to organize the cytoskeleton and signal transduction apparatus of the immune gateway.

CLASP-4 functions in cells of the immune system, e.g., T cells and B cells, as well as non-immune cells. The CLASP-4 protein functions in a variety of cellular processes, particularly related to immune function, regulation of T cell and B cell interactions, T cell activation, and in the organization, establishment and maintenance of the “immunological synapse” (see Dustin et al., 1999, Science 283: 680-682; Paul et al., 1994, Cell 76: 241-251; Dustin et al., 1996, J. Immunol. 157: 2014; Dustin et al., 1998, Cell 94: 667), including signal transduction, cytoskeletal interactions, and membrane organization.

Without intending to be bound by a particular mechanism or limited in any way, the CLASP-4 protein is believed to be a component of the lymphocyte organelle called the “immune gateway” that creates a docking site or portal for cell-cell contact during antigen-presentation. It is believed the cytoplasmic domains of CLASP-4 proteins organize it into a patch at the leading edge of T cells. The carboxy-terminus encoded sequences mediate interaction with cytoskeletal proteins (e.g., spectrin or ankyrin) to connect CLASP-4 to the microtubule network and hold the receptors at a polarized configuration just above the microtubule-organizing center (“MTOC”). Thus, when T cells engages a B cell acting as an APC, the CLASP-4 molecules engage one another to dock the two cells and organize the immune synapse.

Modulating the expression of the CLASP-4 protein, and interference with, or enhancement of, CLASP-4 protein interactions with other proteins has a number of beneficial physiological effects, e.g., altered signaling in response to antigen, altered T and B cell response to antigen, and modulation of T cell activation. In one aspect, the CLASP-4 extracellular domain is targeted (e.g., using anti-CLASP-4 antibody, soluble CLASP-4 fragments, and the like) to regulate T cell activation (and thus regulate immune responses). Disorders that can be treated by disrupting CLASP-4 function, include without limitation, multiple sclerosis, juvenile diabetes, rheumatoid arthritis, pemphigus, pemphigoid, epidermolysis bullosa acquista, lupus, endometriosis, toxemia or pregnancy induced hypertension, pruritic urticarial papules and plaques of pregnancy (PUPPP), herpes gestationis, impetigo herpetiformis, pruritus gravidarum, placenta-related disorders, and Rh incompatibility.

In another aspect, the present invention provides methods and reagents for detection of CLASP-4 expression and CLASP-4-expressing cells. Abnormal expression patterns or expression levels are diagnostic for immune and other disorders. For example, diseases characterized by overproduction or depletion of lymphocytes in blood or other organs may be detected or monitored by monitoring the level of CLASP-4 polypeptide or mRNA in a biological sample (e.g., peripheral blood), e.g., the number or percentage of CLASP-4 expressing cells. Diseases characterized by overproduction of T cells include, e.g., leukemia (both ALL and CLL), lymphoma (including non-Hodgkins lymphoma, Burkitt's lymphoma, mycosis fungoides, and sezary syndrome), EBV, CMV, toxoplasmosis, syphilis, typhoid, brucellosis, tuberculosis, influenza, hepatitis, serum sickness, and thyrotoxicosis. Diseases associated with the depletion of T cells include, e.g., HIV and myelodysplasia. Diseases associated with the overproduction of B cells include, e.g., leukemia (both ALL and CLL), non-Hodgkins lymphoma, Burkitt's lymphoma, myeloma, EBV, CMV, toxoplasmosis, syphilis, typhoid, brucellosis, tuberculosis, influenze, hepatitis, serum sickness, and thyrotoxicosis. Diseases associated with the depletion of B cells include, e.g., myelodysplasia.

CLASP-4 CDNA and Polypeptide Structure

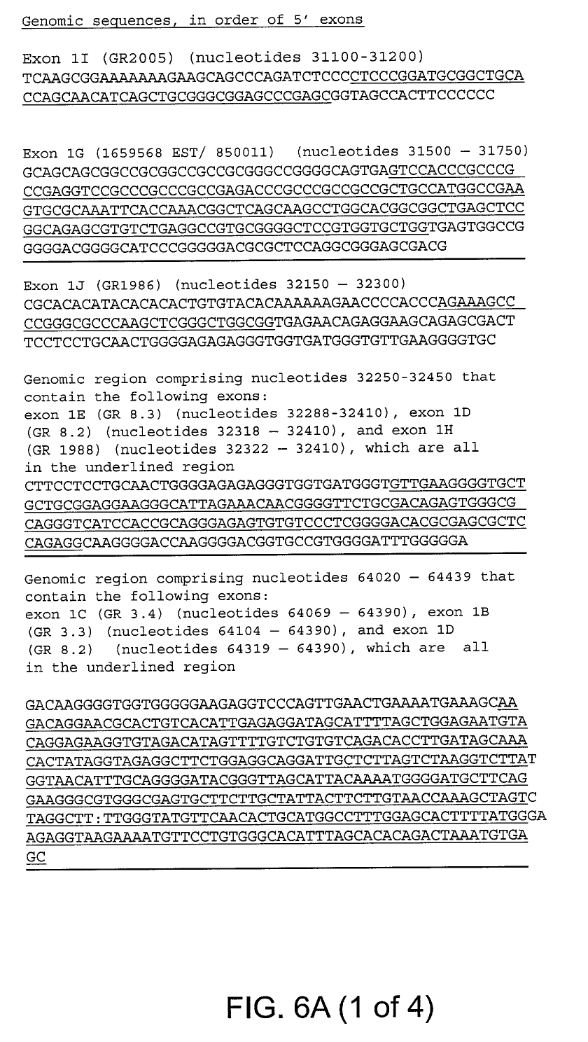

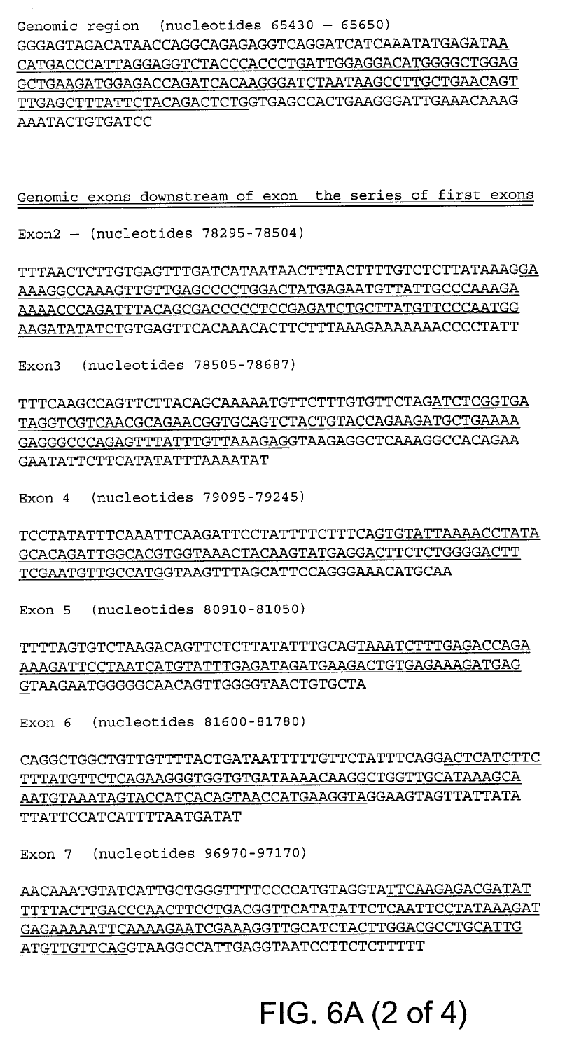

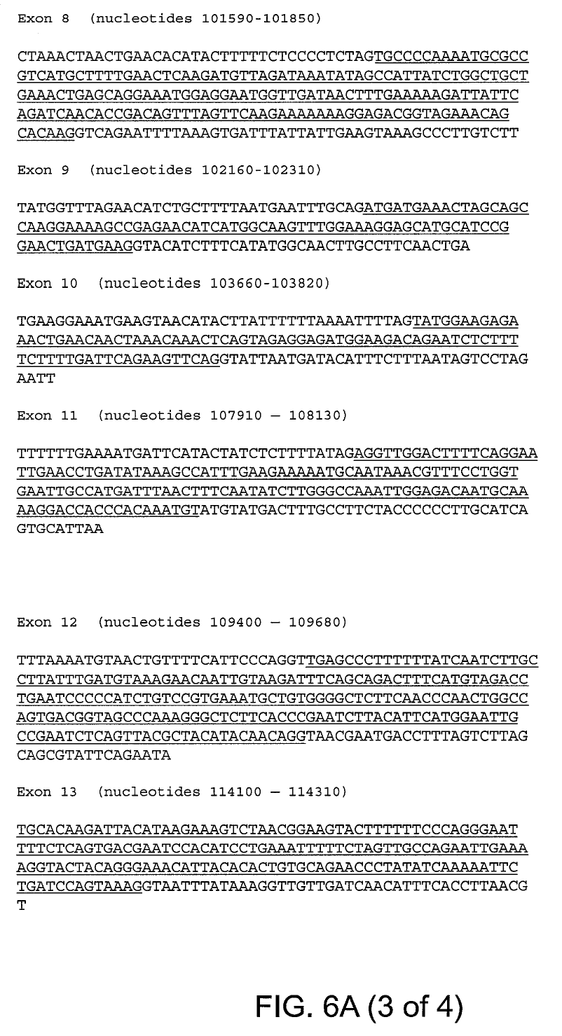

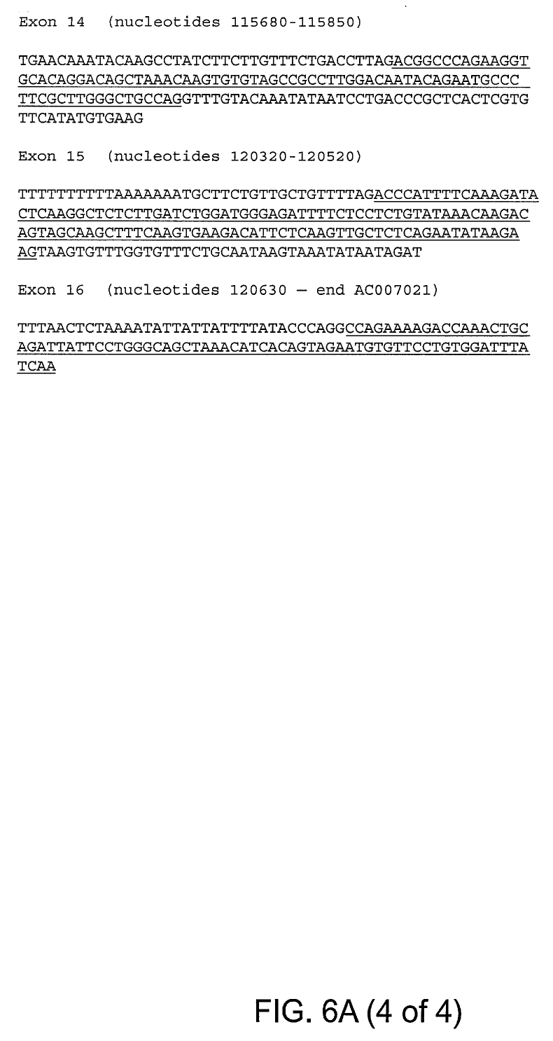

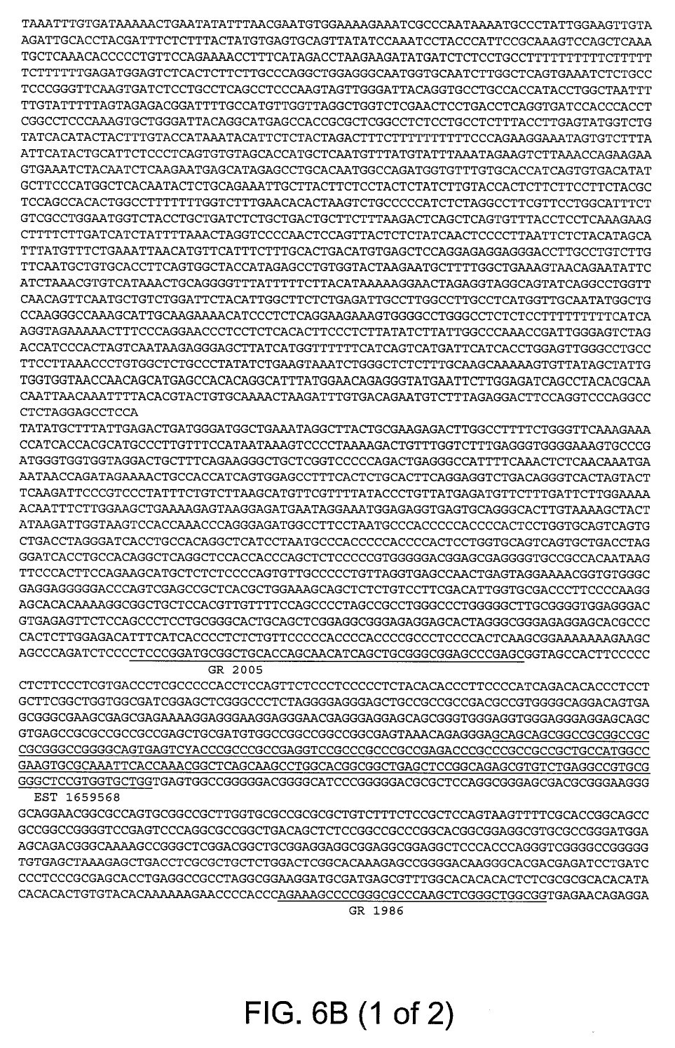

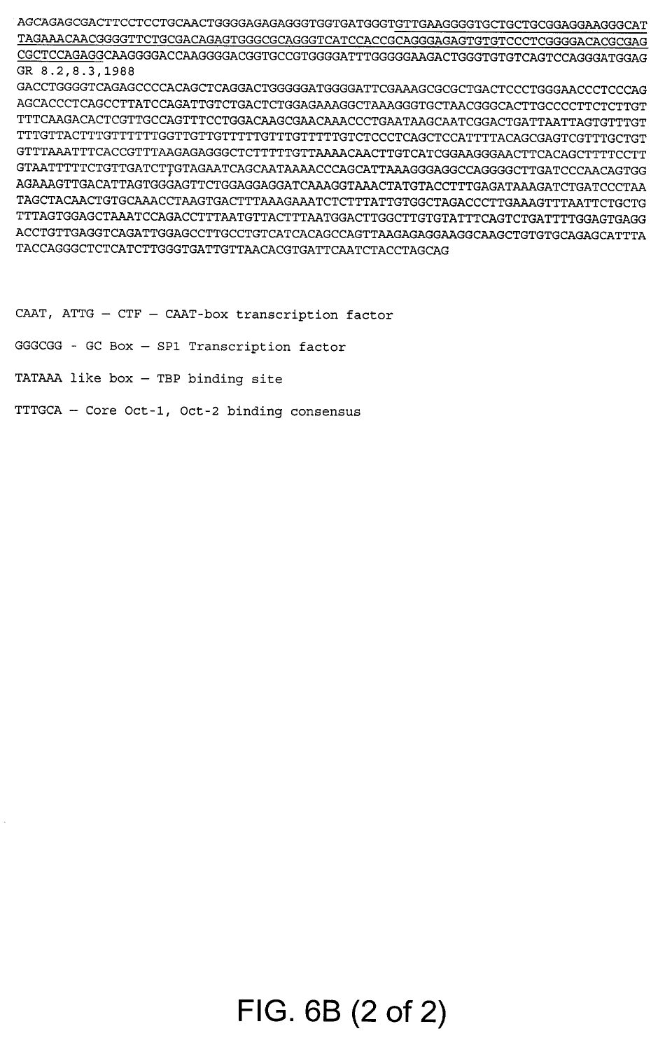

The CLASP-4 protein is type I transmembrane glycoprotein. FIG.6. shows the nucleotide sequence and conceptual translation of human CLASP-4 polypeptide:

hCLASP-4 cDNA (SEQ ID NO: 1) and hCLASP-4 polypeptide (SEQ ID NO:2).

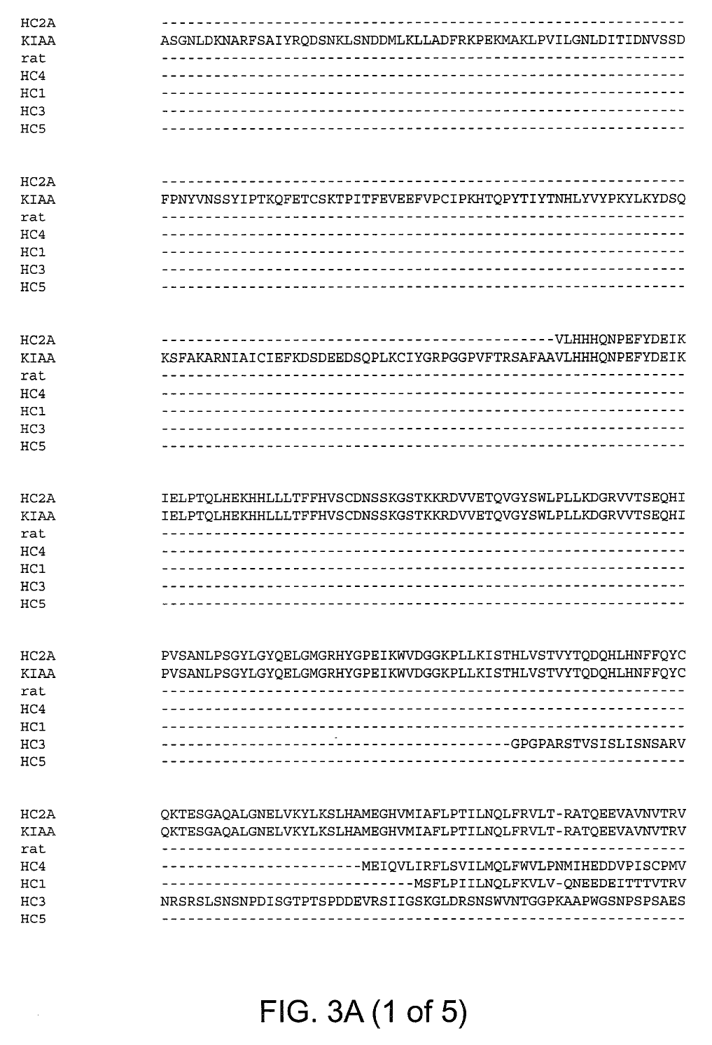

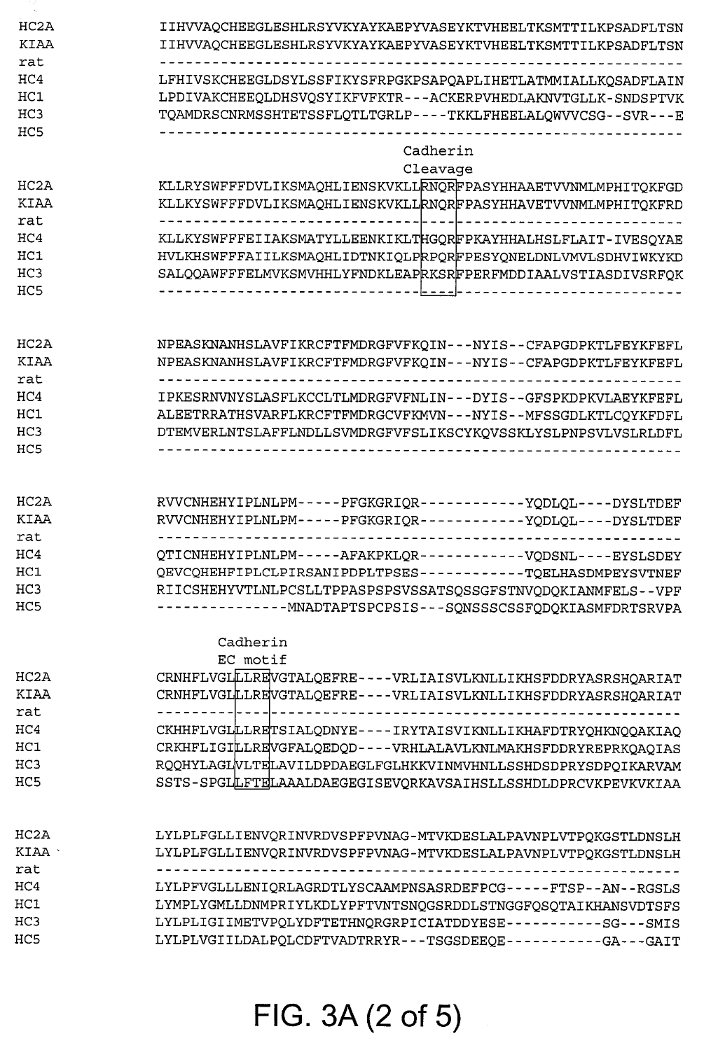

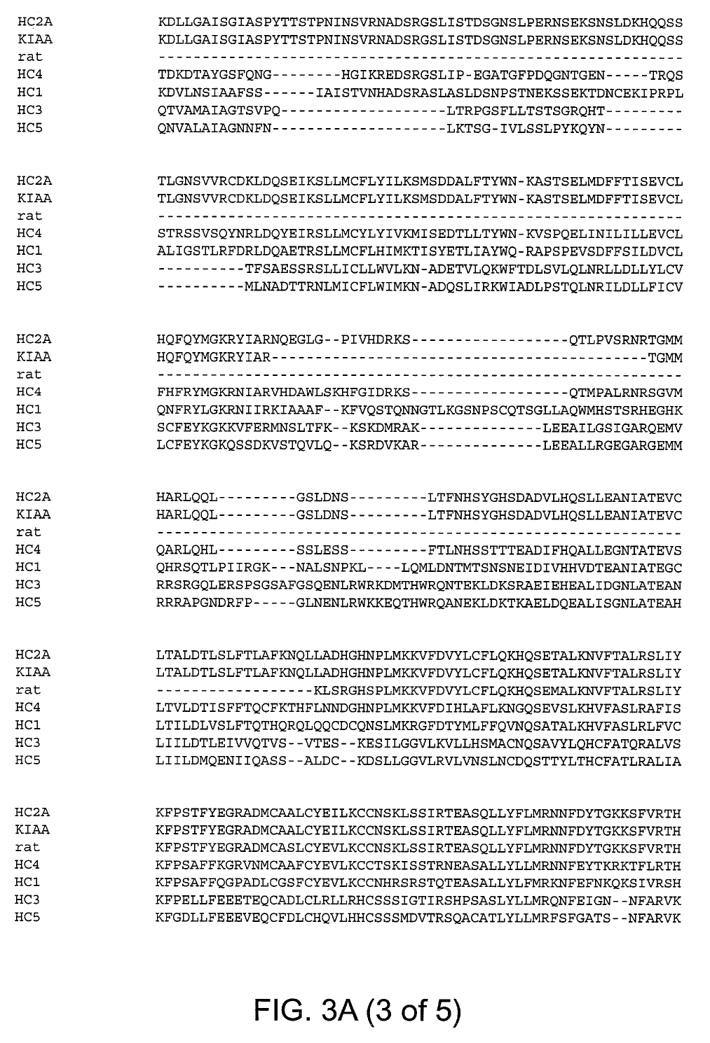

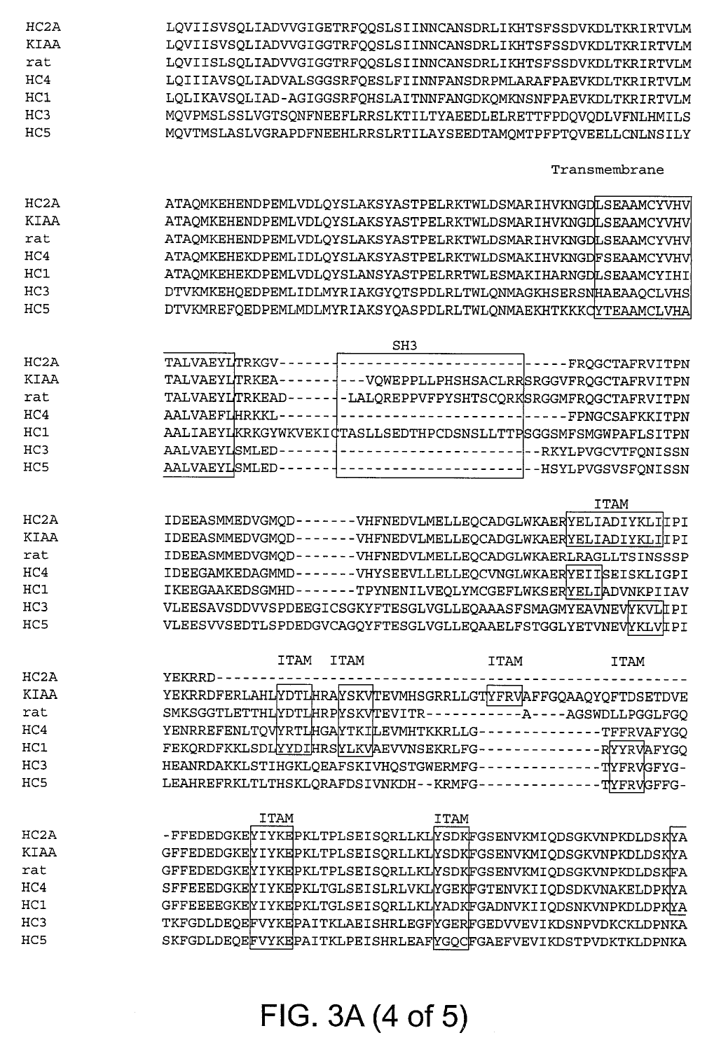

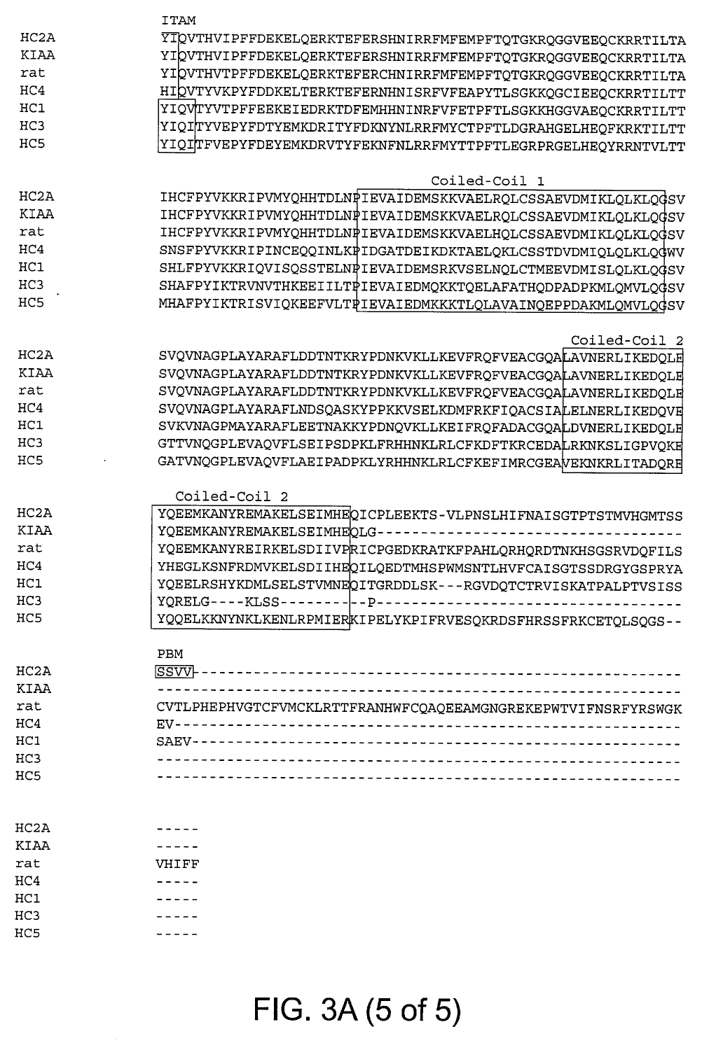

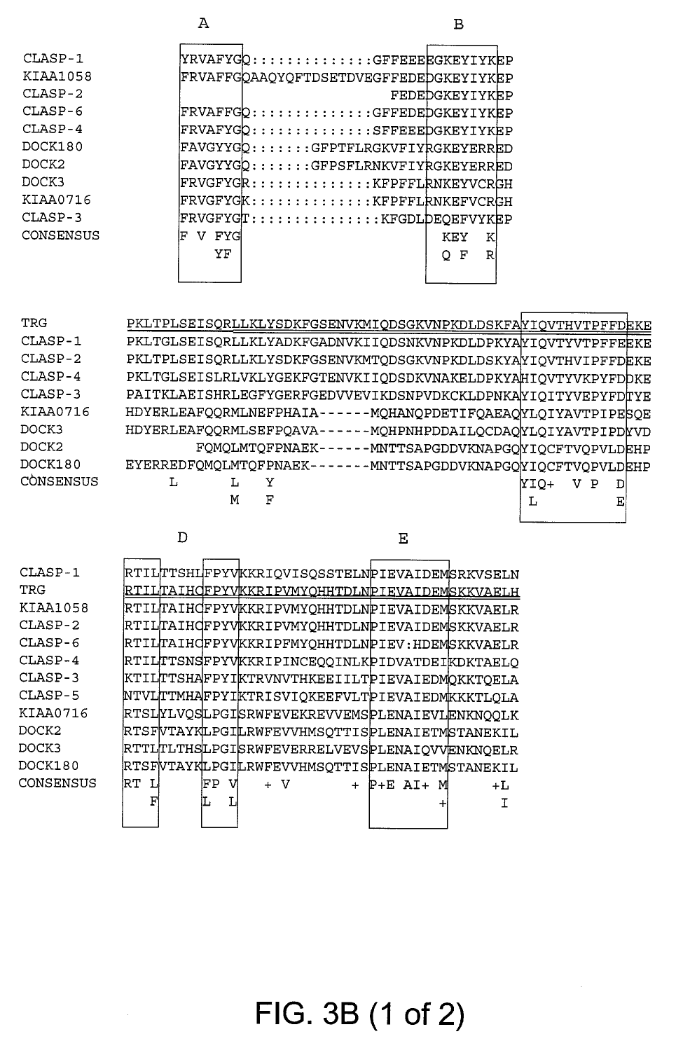

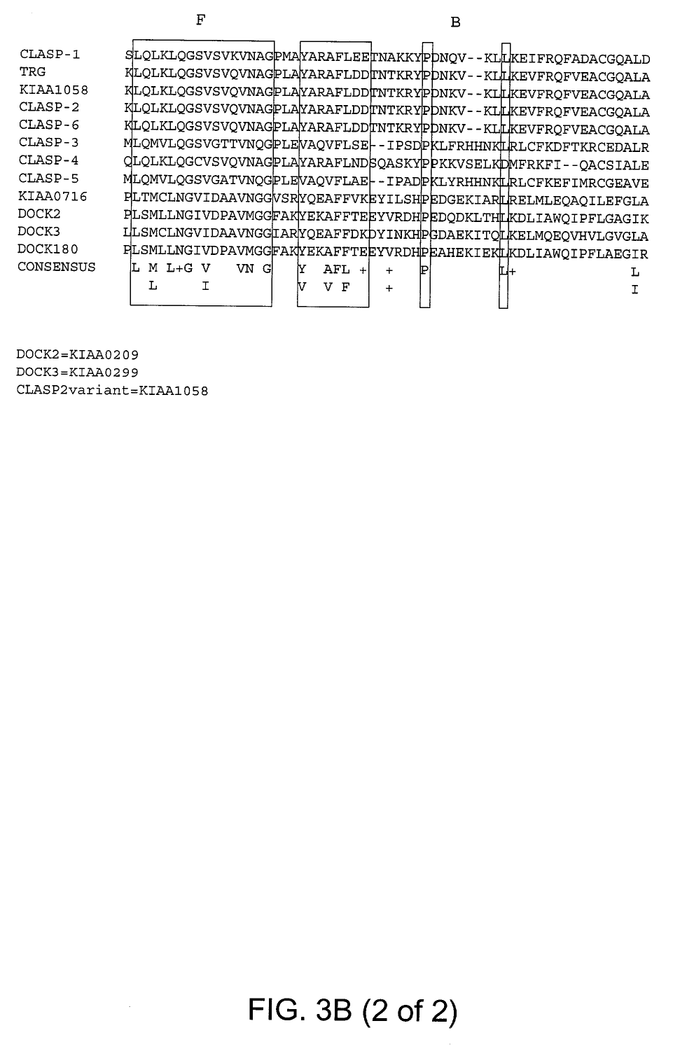

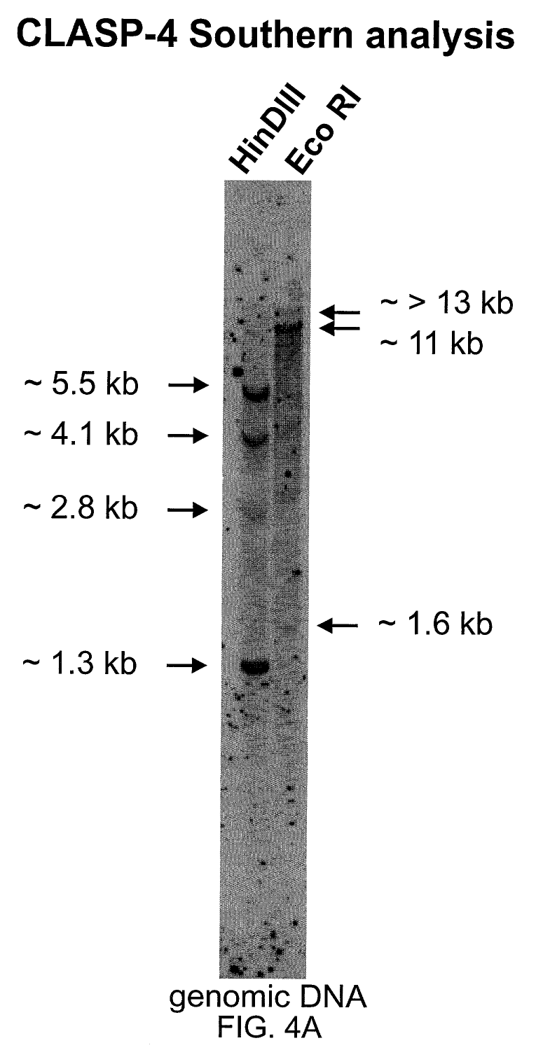

The phrase “human CLASP-4 (hCLASP-4)” as used herein refers to hCLASP-4. As shown in FIG. 3, “KIAA” is KIAA1058 sequence (Genbank Accession No. AB028981), which was described by Kikuno et al., 1999, DNA Res. 6, 197-205 as a cDNA from brain encoding a protein of unknown function.

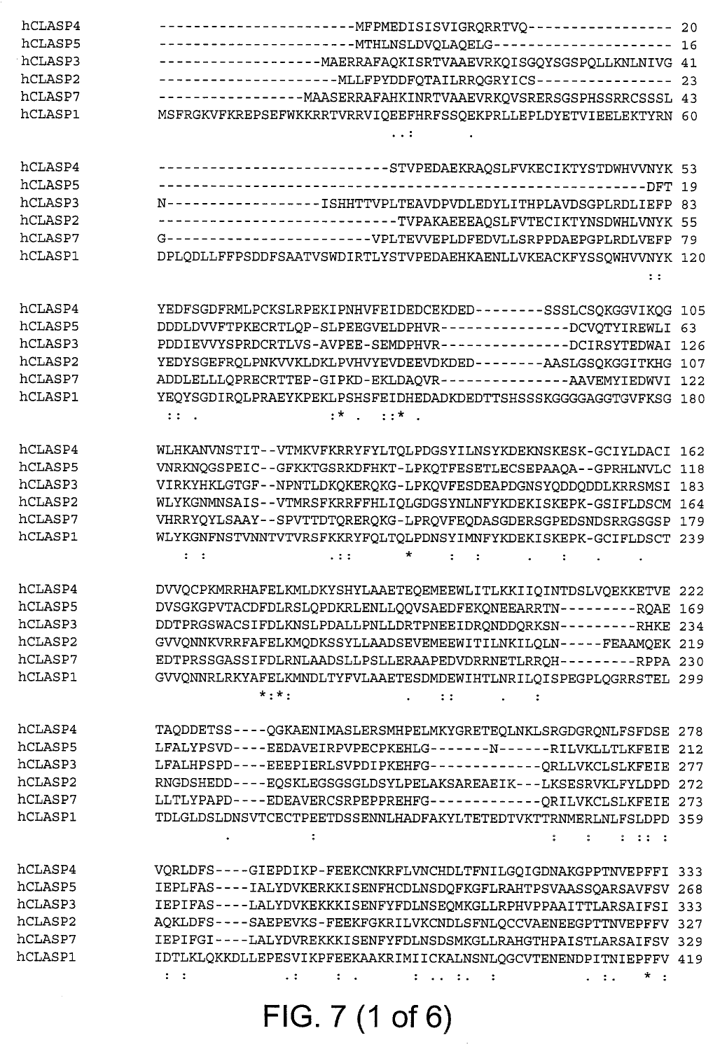

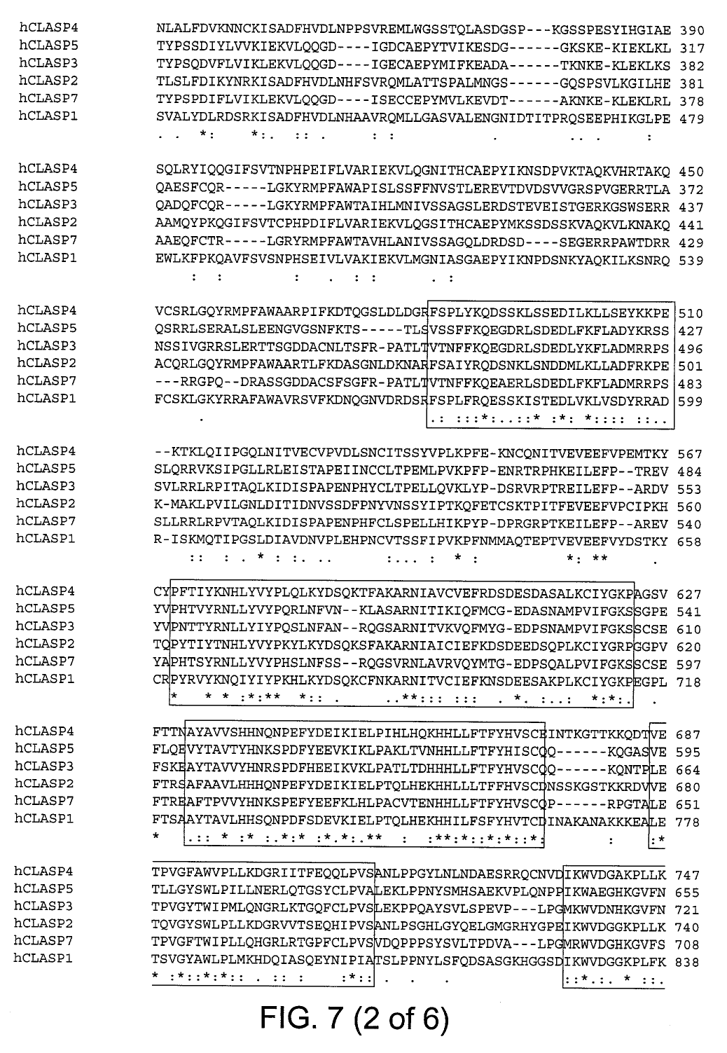

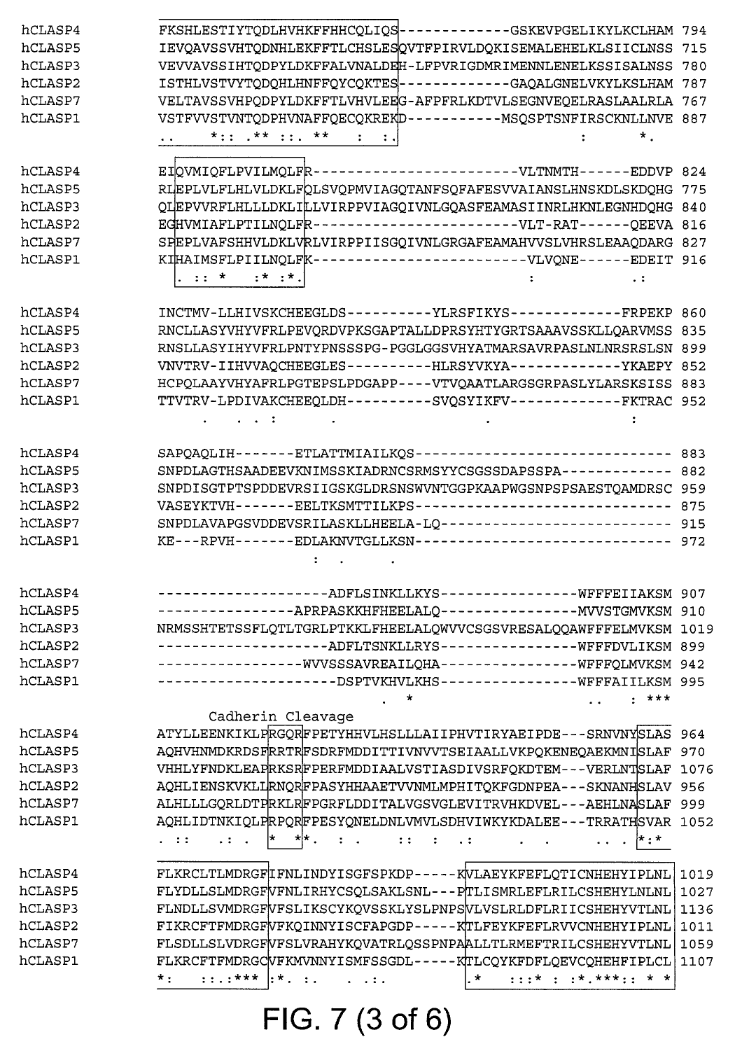

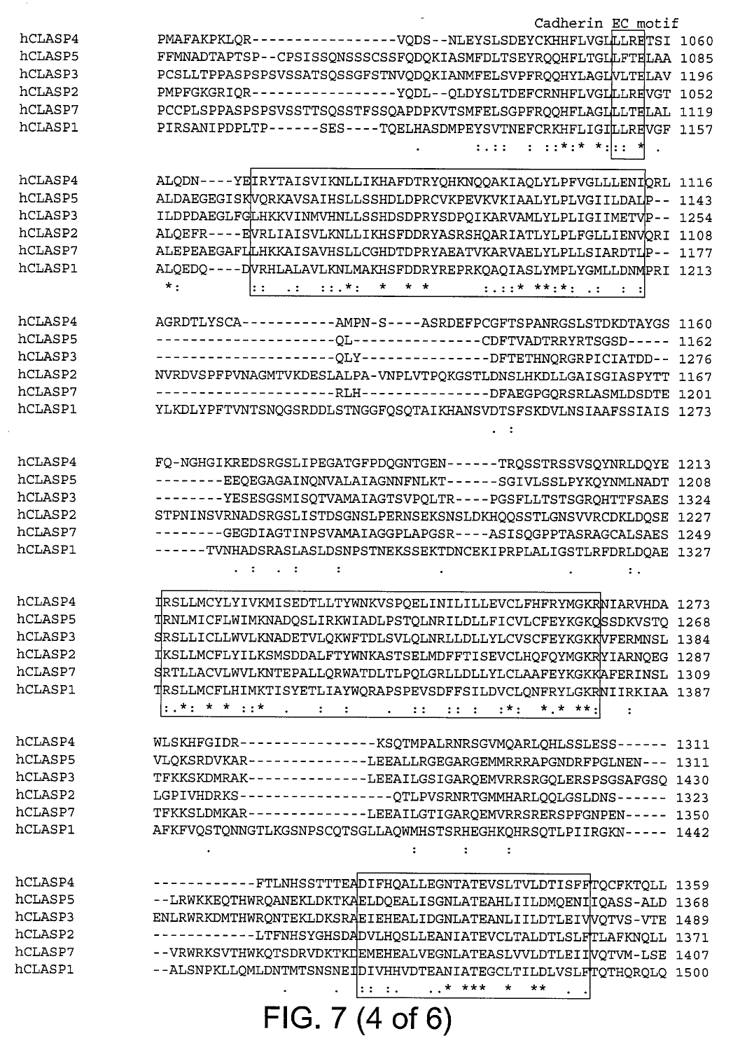

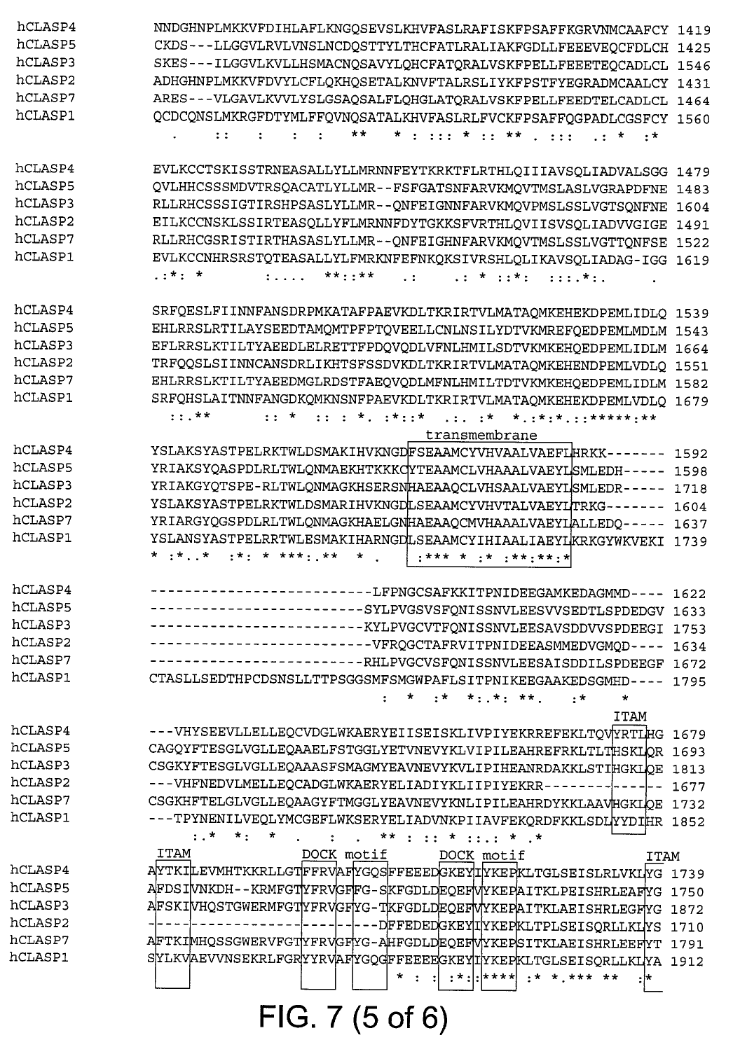

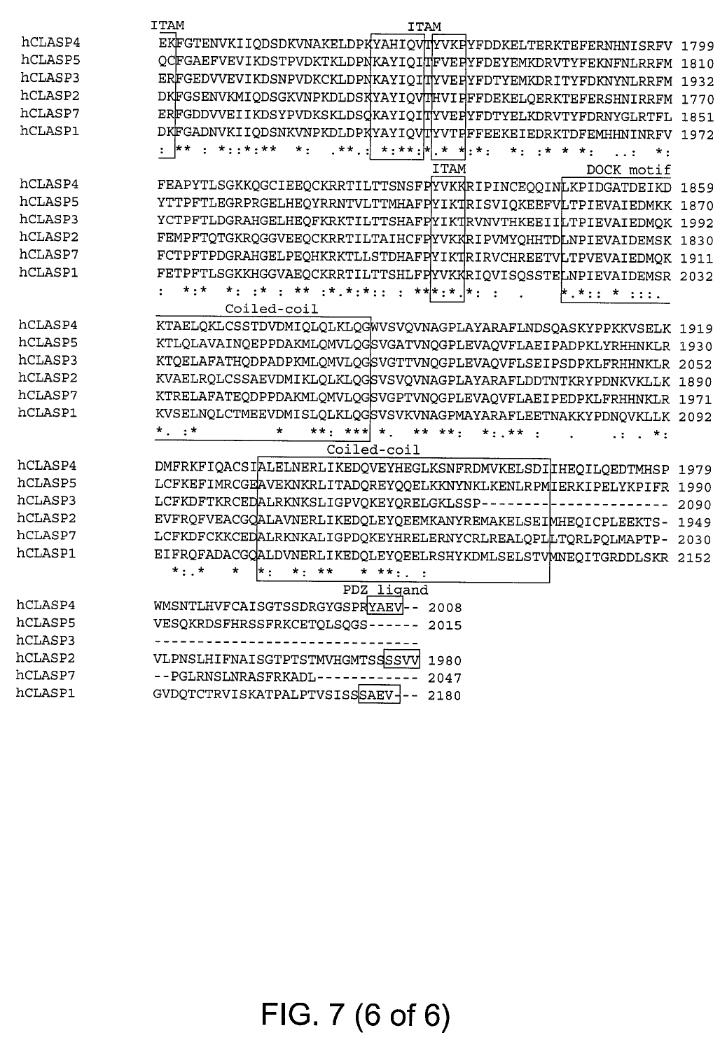

CLASP-4 polypeptides typically include a cadherin proteolytic cleavage signal RXXR, a transmembrane domain (amino acids 1570-1588 in FIG. 7) and an intracellular domain. Immediately adjacent to the transmembrane domain is an extracellular portion of CLASP-4. However, there are additional hydrophobic regions in the region encompassing amino acids 1-1569 that may be potential membrane spanning regions. CLASP-4 therefore contains at least 1 but possibly more transmembrane domains. Standard techniques are available to determine the topology of a protein including cysteine accessibility analysis (see, e.g., Wakabayashi S. et al., 2000, J. Biol. Chem. 275:7942-9); epitope tagging of proteins (see, e.g., Gruarin P., 2000, Biochem Biophys Res Commun 275:446-54; Harms N., 1999, J. Mol. Microbiol. Biotechnol. 1:319-25); and trypsin sensitivity (see, e.g., da Fonseca F. et al., 2000, J. Virol 74: 7508-17). The present invention provides a polynucleotide having the sequence of SEQ. ID. NO: 1, or a fragment thereof, and a polypeptide having the sequence of SEQ. ID NO:2, or a fragment thereof. In addition, the invention provides polynucleotides comprising hCLASP-4 genomic sequences, CLASP-4 homologs from other species, naturally occurring alleles of hCLASP-4, and hCLASP-4 variants as described herein, and methods for using CLASP-4 polynucleotide, polypeptides, antibodies and other reagents.

CLASP-4 Polypeptide Domains

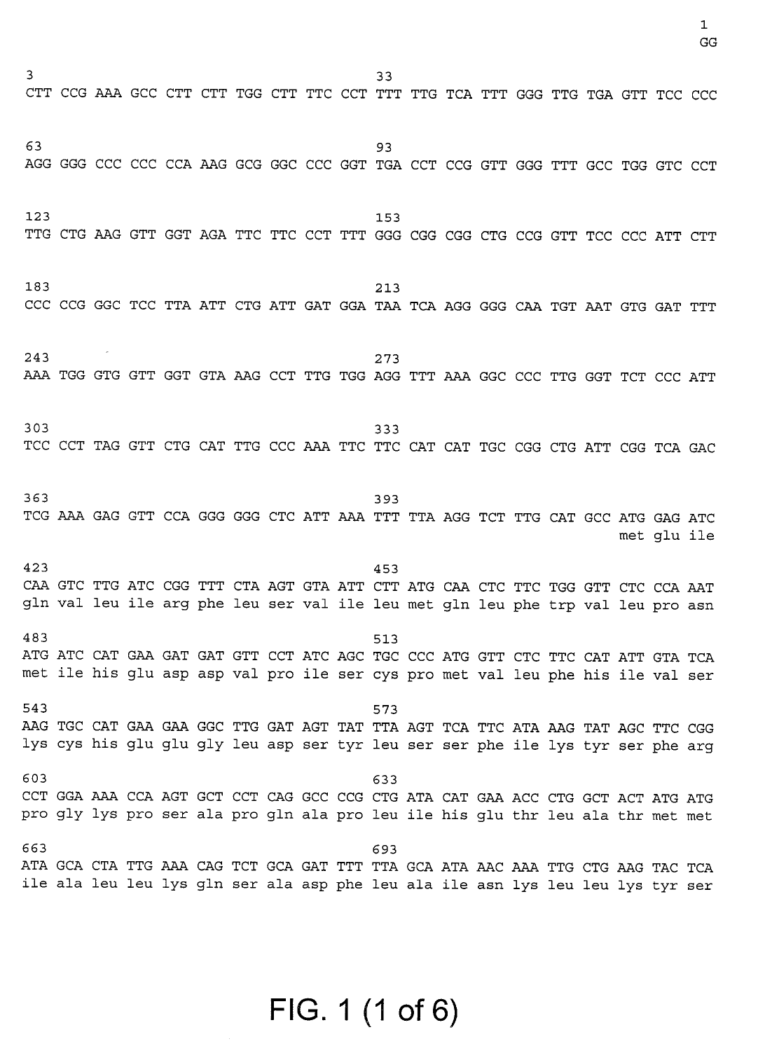

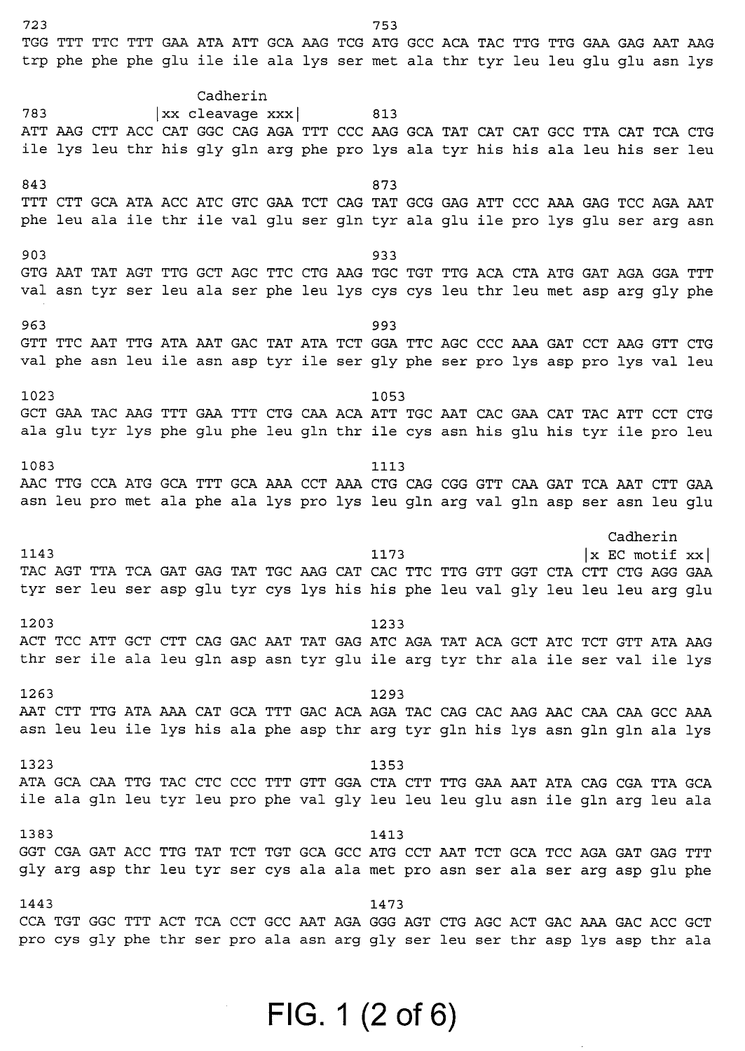

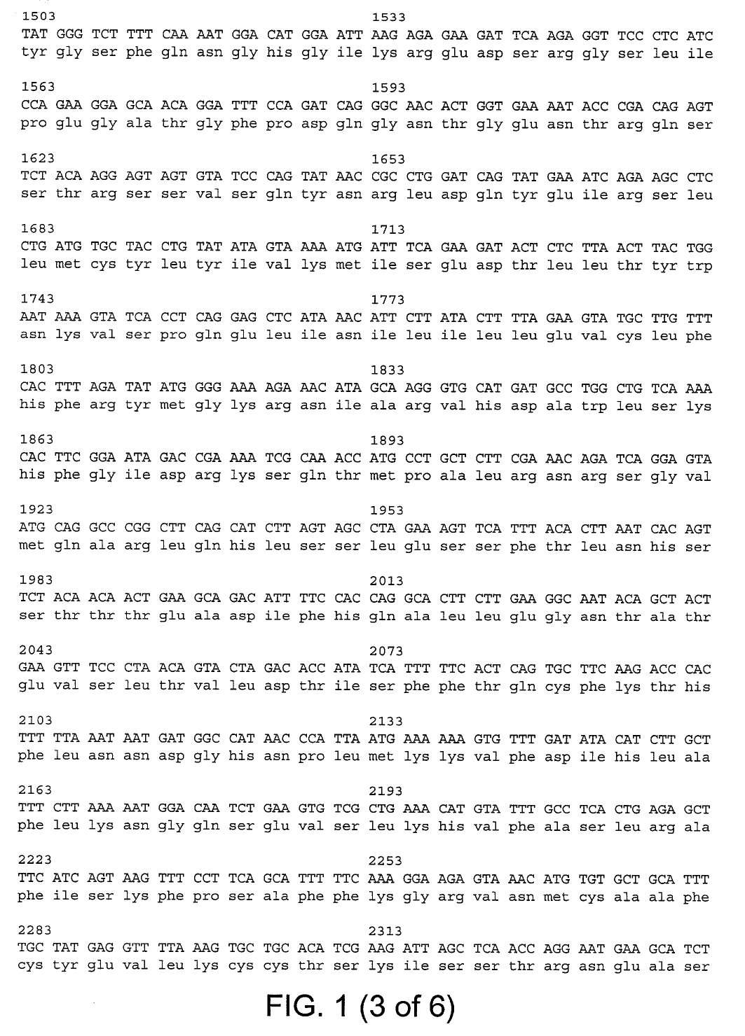

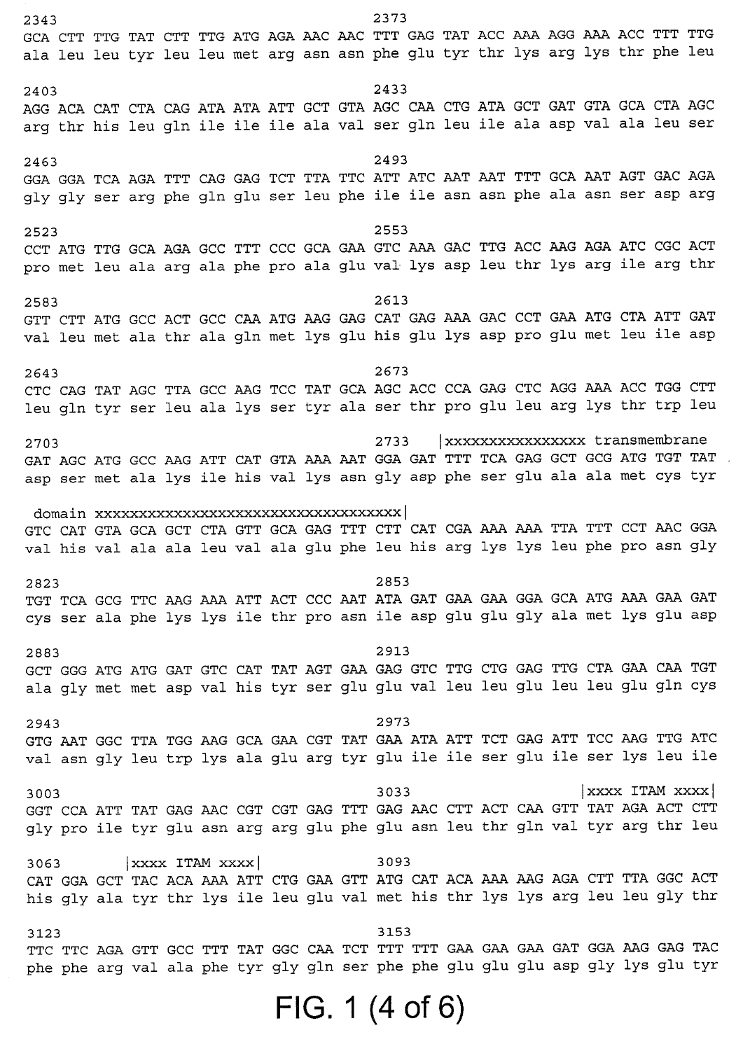

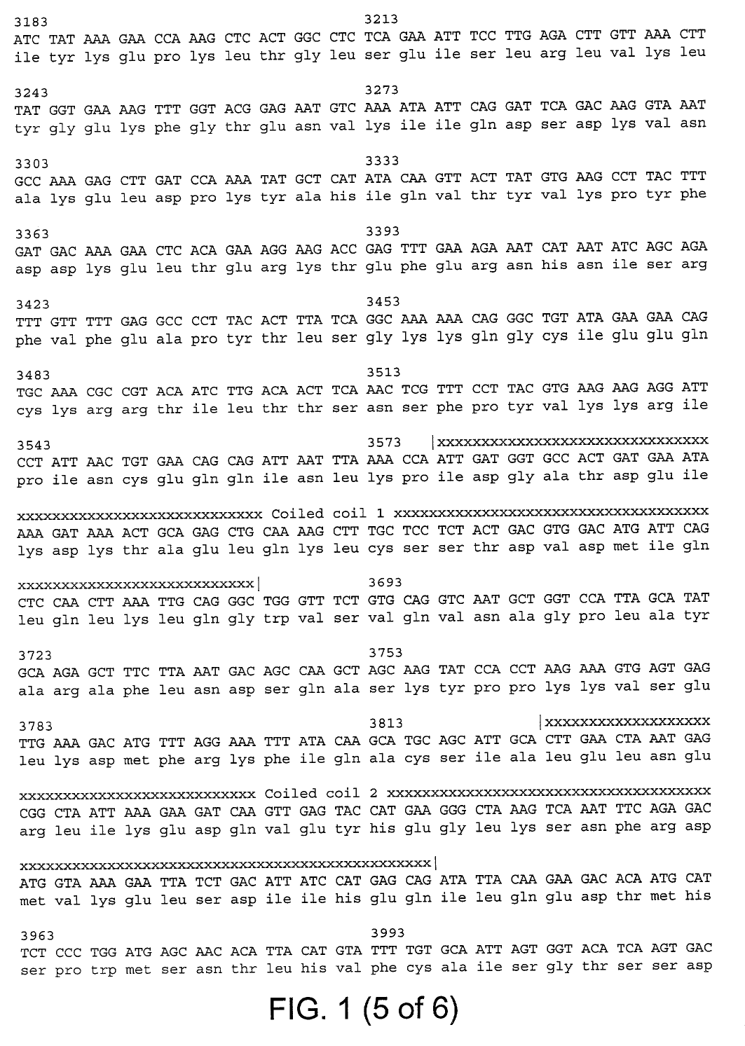

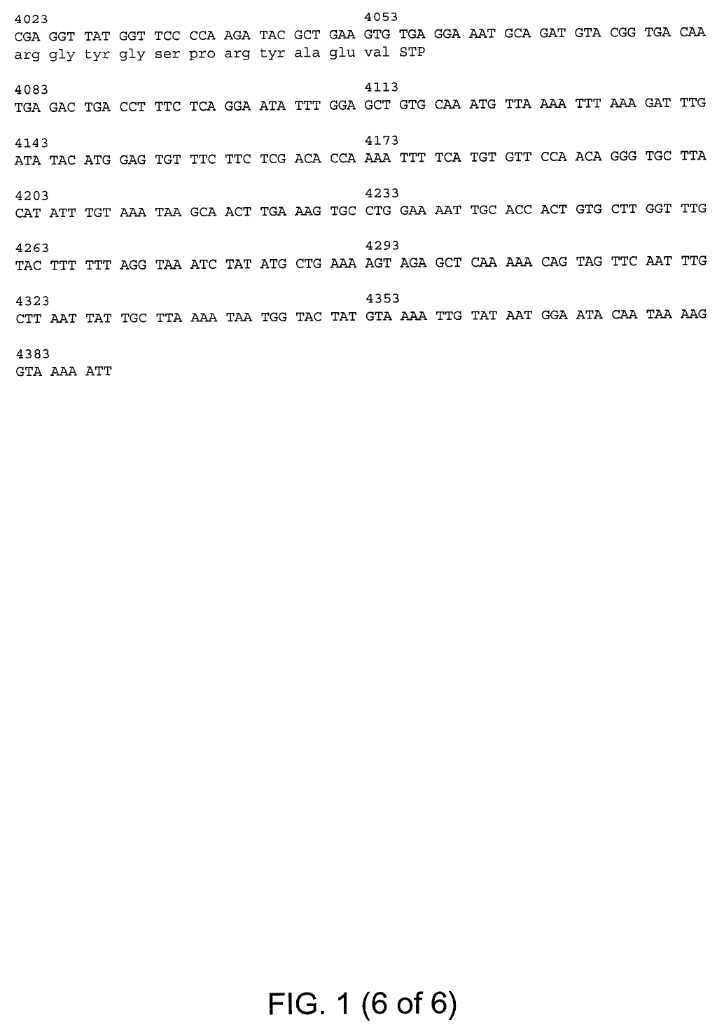

As is shown in FIG. 1, one naturally occurring CLASP-4 cDNA encodes a polypeptide characterized by several structural and functional domains and defined sequence motifs. To provide guidance to the practitioner, the structural features are described infra. However, it will be understood that the present invention is not limited to polypeptides that include all, or any particular one of these domains or motifs. For example, a CLASP-4 fusion protein of the invention contains only the extracellular domain of CLASP-4. Similarly, the CLASP-4 polypeptide of SEQ ID NO:2 does not have the ITAM motifs (discussed infra) found in the other CLASP family polypeptides.

It will be appreciated that the structurally (and functionally) different domains of CLASP-4 polypeptides (and the corresponding region of the mRNA) are of interest, in part, because they may be separately targeted or modified (e.g., deleted or mutated) to affect the activity or expression of a CLASP-4 gene product (in order to, for example, modulate an immune response). For example, the extracellular domain of a CLASP-4 protein can be targeted (e.g., using an anti-CLASP monoclonal antibody to (a) block the interaction of a CLASP-4-expressing cell (e.g., a T cell) and a second cell (e.g., a B cell) displaying a protein that is bound by CLASP-4 (i.e., a CLASP-4 ligand). Similarly, an intracellular domain (e.g., DOCK, see infra) can be targeted to interfere with signal transduction without interfering with extracellular ligand binding.

Generally, inhibiting CLASP-4 expression or CLASP-4 polypeptide function will result in modulation of immune function including, for example, changing the threshold for T cell activation by affecting formation of the immune synapse. Modulation of immune function can be screened and quantitated by a number of assays known in the art and described herein (see also “Biological Activities of CLASP-4” subsection below).

### Signal Peptide

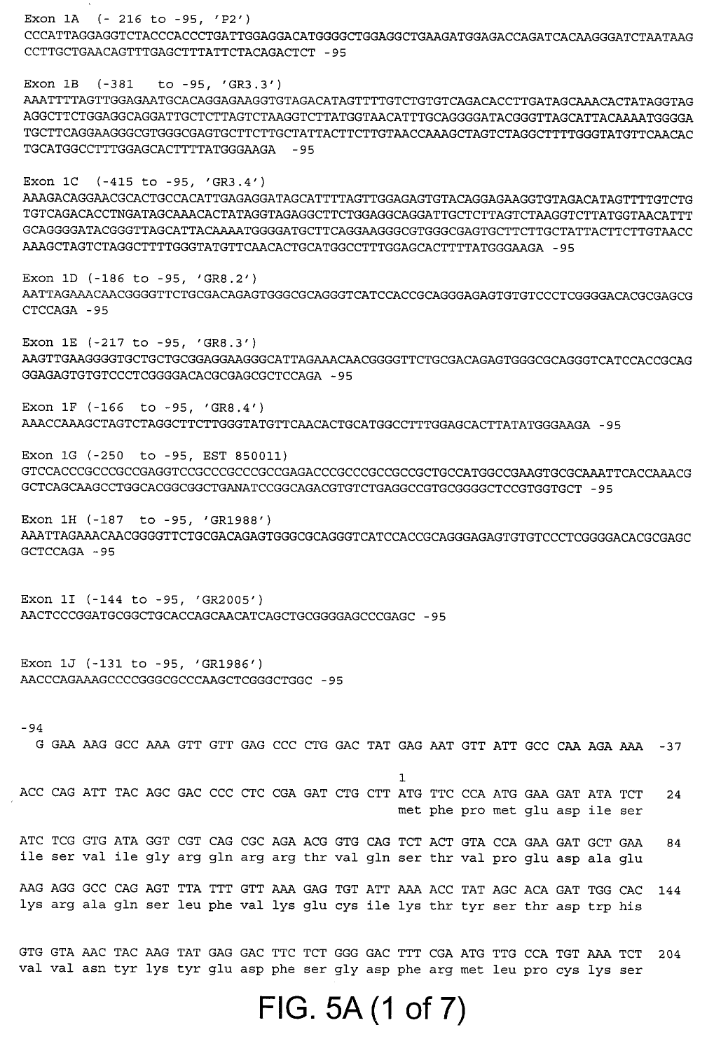

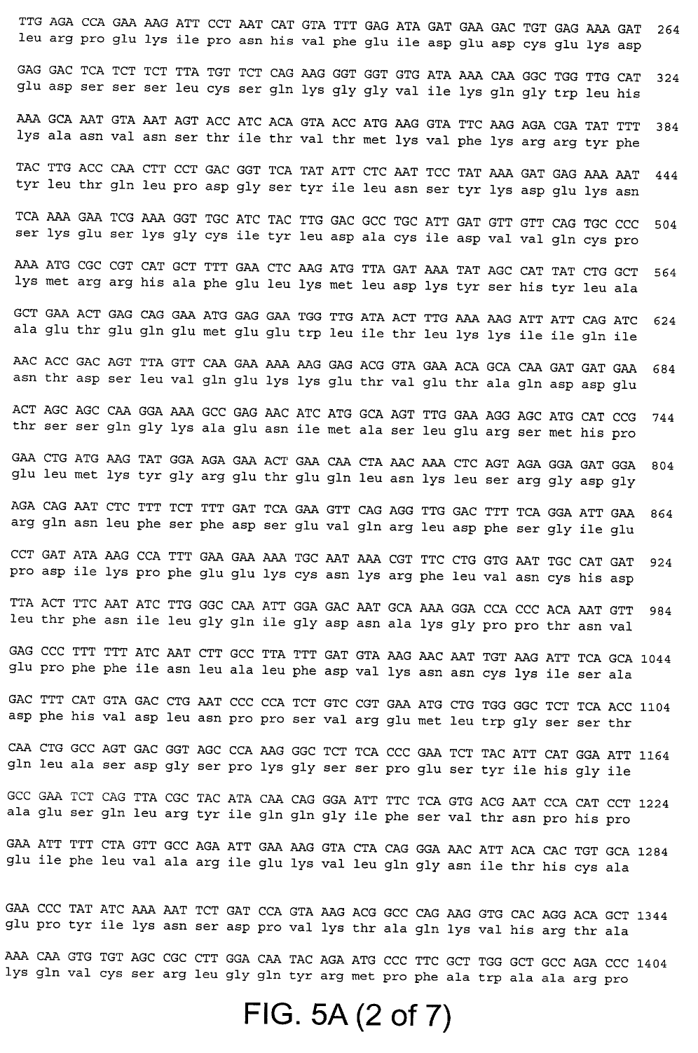

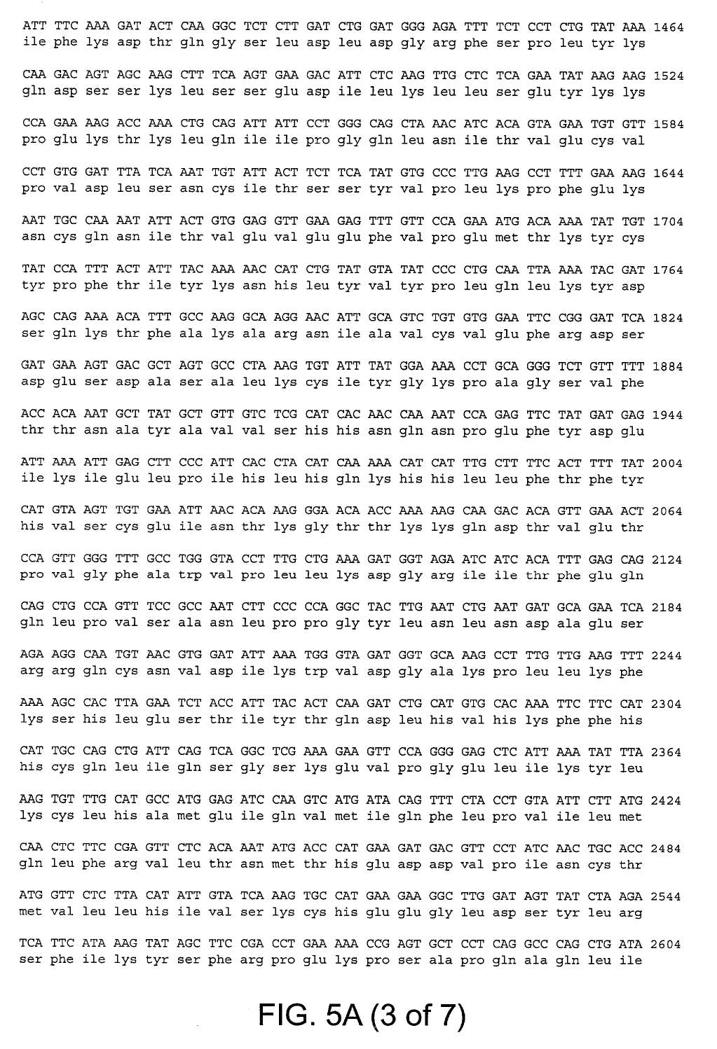

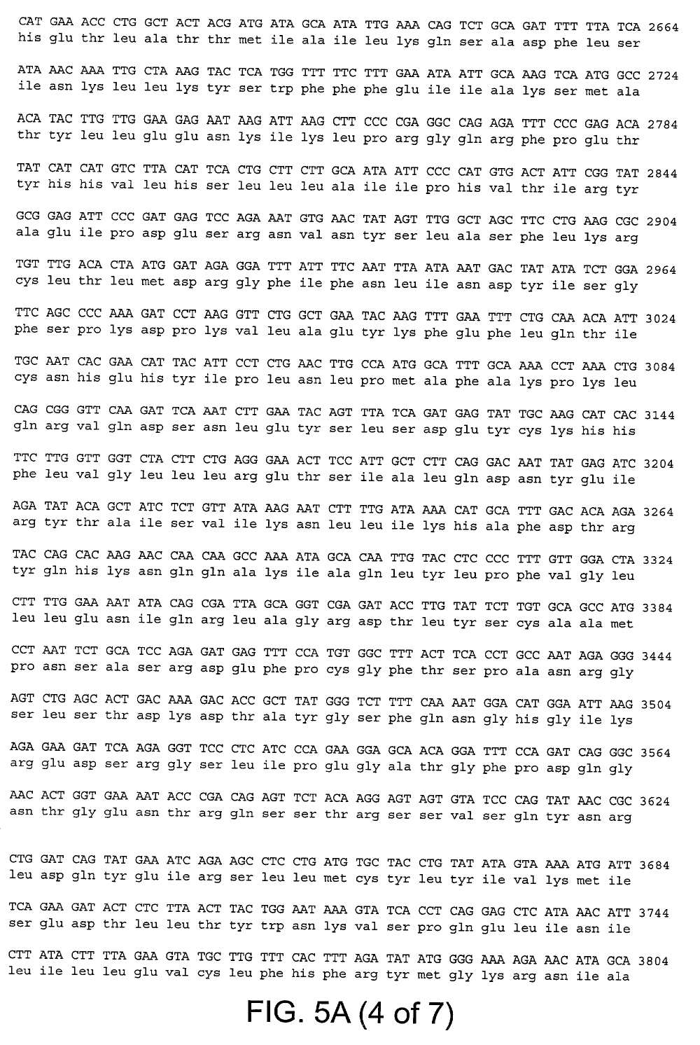

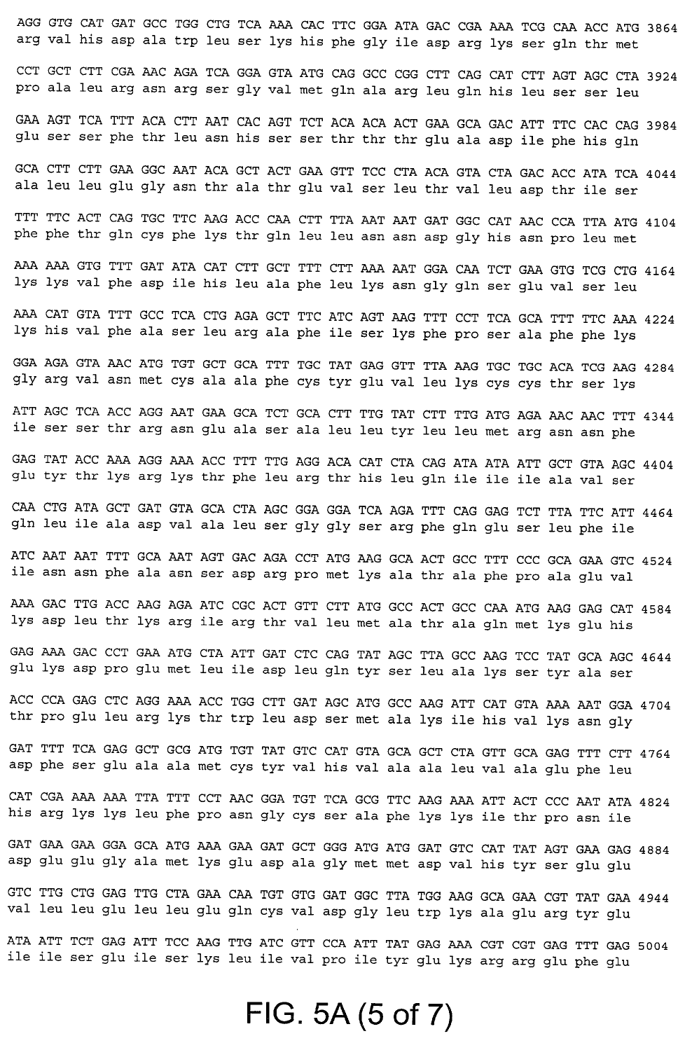

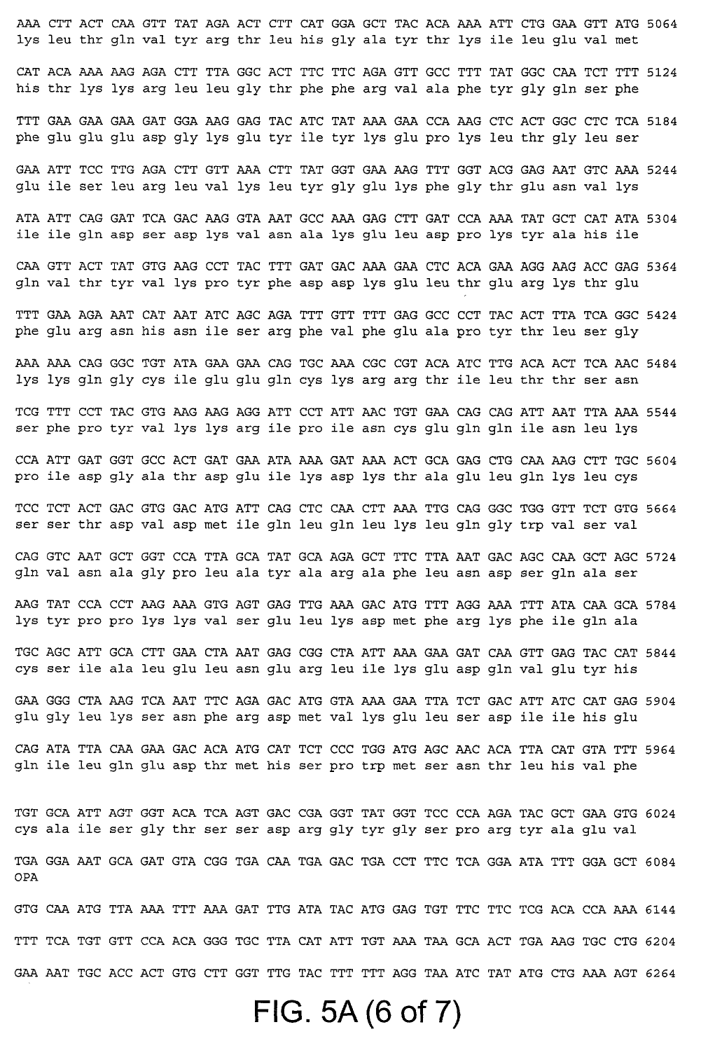

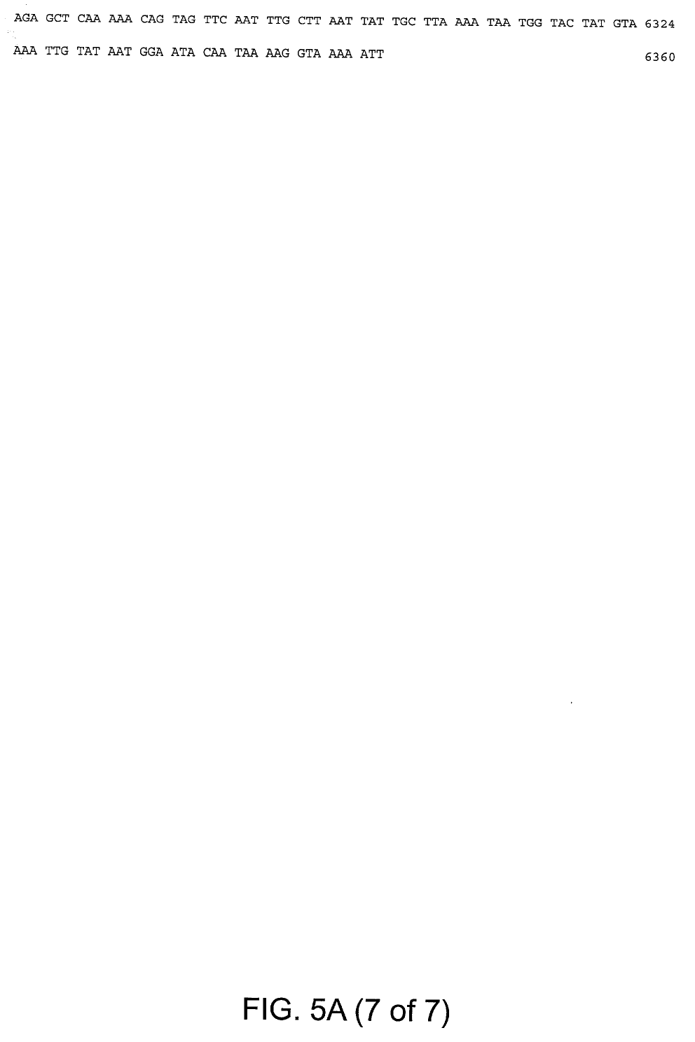

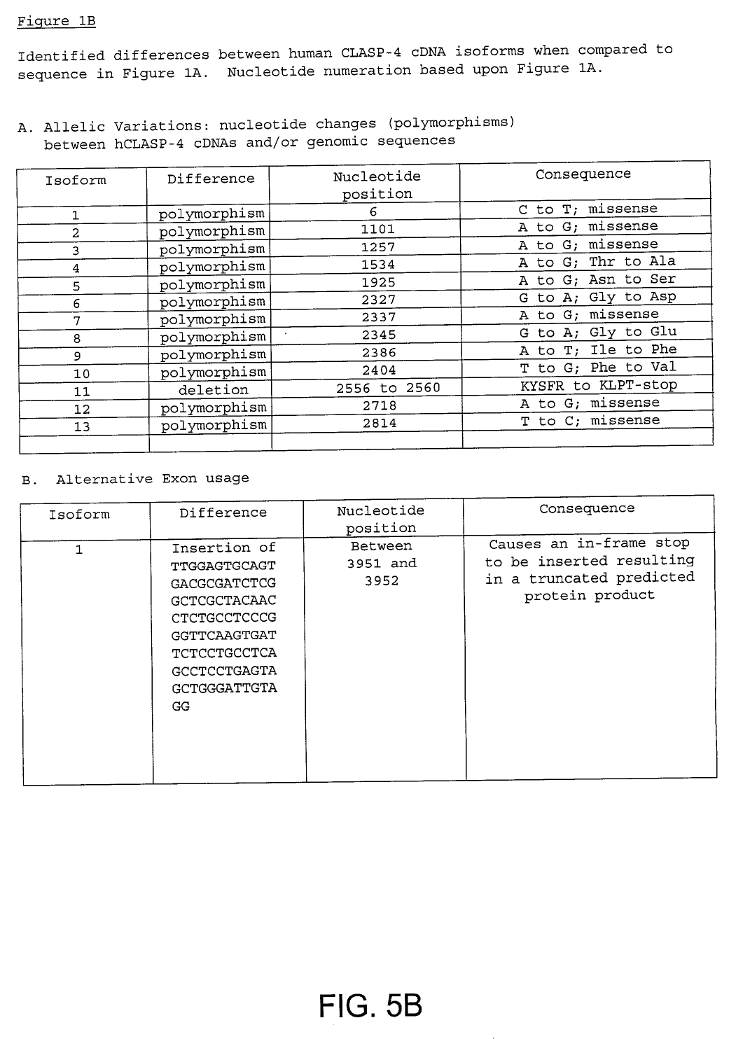

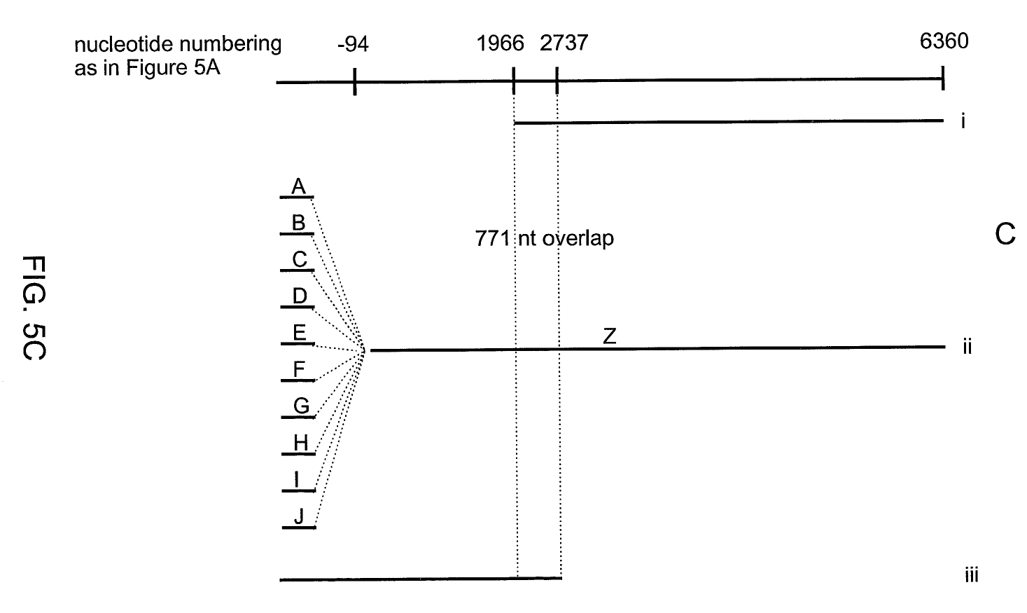

The human CLASP-4 sequence presented in FIG. 1 shows a potential start site for translation. The predicted methionine appears at nucleotide 414 (ATG). It is an acceptable consensus sequence for a translational start (A/GxxATGG; Kozak, M., 1996, Mamm. Genome 7(8): 563-74). A second possibility for a translational start is that the cDNA listed in FIG. 1 is incomplete and another methionine is encoded in frame and upstream of the sequence shown in FIG. 1. Additional CLASP-4 cDNA sequence is shown in FIG. 5 and contains an initiator methionine at nucleotide +1. Sequences 5′ to the ATG at nucleotide 414 in FIG. 1 were obtained by sequencing a genomic BAC clone and could represent either upstream intron sequence or an exon not normally found in expressed cDNAs.

### Extracellular Domain

The CLASP-4 extracellular domain is characterized by one cadherin EC-like motif (Pigott, R. and Power, C., 1993, The Adhesion Molecule Factbook. Academic Press, pg. 6; Jackson, R. M. and Russell, R. B., 2000, J. Mol. Biol. 296: 325-34). Several highly conserved cysteines are found in the extracellular domain, as well as various glycosylation signals. Through its extracellular domains, CLASP-4 may interact with ligands in a homotypic and/or heterotypic manner to establish the immunological synapse in conjunction with molecules such as TCR, MHC class I, MHC class II, CD3 complex and accessory molecules such as CD4, CD3, ICAM-1, LFA-1, and others. Many cadherins contain a pro-domain of approximately 50 to 150 amino acids that is removed before localization to the plasma membrane. This cleavage is presumed to be carried out by Furin (Posthaus, H. et al., 1998, FEBS Let 438: 306-10) at a consensus sequence of RKQR. Furin is a protease that is at least partially responsible for the maturation of certain cadherins. CLASP-4 has the sequence HGQR at nucleotides 795 through 806 as shown in FIG. 1 (nucleotides 2761 through 2772 that encodes the sequence RGQR as shown in FIG. 5). By homology, this region is around 120 amino acids into the predicted protein start site for hCLASP-4. This region may be a pro-domain and cleavage may be required for CLASP-4 function, or aspects of CLASP-4 function.

Antibodies raised against the extracellular domain can be added to cells expressing CLASP-4. These antibodies can either block the interaction of CLASP-4 with potential ligands or stabilize these interactions. Any immunoassay known in the art, e.g., listed and described herein, may be used to assess the modulation of immune function brought about by this approach.

Similarly, portions of the extracellular domain of CLASP-4 can be expressed as soluble protein. This soluble protein can then be added to cells expressing CLASP-4. These proteins may interact with potential ligands to competitively inhibit their binding to endogenous CLASP-4. This could modulate CLASP-4 function via the immunoassays described herein. Recombinant proteins could interfere in a positive or negative fashion with CLASP-4 interactions.

### Transmembrane Domain

CLASP-4 predicted amino acid sequence was analyzed using the PHDhtm analysis software for prediction of transmembrane helices (Rost, B., et al., 1996, Prot. Science 7: 1704-1718). Using the PPHDhtm analysis software, it was determined that a transmembrane domain is located from nucleotides 2739-2795 (as shown in FIG. 1; nucleotides 4708 to 4764 as shown in FIG. 5 Other potential transmembrane domains located in the amino terminal 1569 amino acids (as shown in FIG. 5) are possible.

### Intracellular Domains

The CLASP intracellular domains contain motifs corresponding to several types of protein domains. Depending on the specific CLASP (i.e., specific family member or splice variant) all or only some of the domains can be present. Listed from amino terminus to carboxy terminus, the domains include: (1) ITAM (Chan et al. 1994, Annual Review of Immunology 12: 555-592), (2) a newly discovered DOCK/CLASP-4 motif, (3) a coiled-coil motif, and (4) a C-terminal PDZ binding motif (PBM) (also referred to as PDZ ligand or “PL”).

### ITAM

Immunoreceptor Tyrosine-based Activation Motifs (ITAM motifs; also known as ARAM, or antigen recognition activation motifs) are motifs contained within antigen receptors for T and B cells, and Fc receptors on other leukocytes, and are necessary for proper activation and signal transduction in these cells. They are characterized by the consensus sequence YXXL/I-X7/8-YXXL/I (Grucza et al., 1999, Biochemistry 38: 5024-5033), usually separated by 6-8 amino acids (Watson et al., 1998, Immunol. Today 19: 260-264; Isakov, J. Leukoc. Biol. 61: 6-16). ITAM is used as an intracellular regulatory motif through its ability to be tyrosine phosphorylated by src-family tyrosine kinases such as Lyn that are involved in leukocyte signal transduction. Once phosphorylated, the ITAM acts as a high affinity binding site for SH2 containing proteins. Signal transduction components including ZAP-70, Syk, Lyn, Shc, PI3 kinase, and Grb2 contain SH2 domains and have been shown to bind ITAMs (Clements et al., 1999, Annu. Rev. Immunol. 17: 89-108). This places ITAM-containing molecules in a central role of intracellular signal regulation in leukocytes. ITAM motifs in leukocyte signaling can facilitate signal transduction (e.g., tyrosine kinase signaling) by acting as temporal scaffolds where other transduction components could bind and be properly positioned to mediate transduction. ITAM motifs often appear in multiples in a protein, however, it is known that one set of YXXL/I alone can transduce signals of the PTK pathway, though weakly.

CLASP-4 proteins typically have ITAM YXXL/I motifs (where X is any amino acid) separated by 3 or 13 amino acids. In various embodiments the CLASP-4 polypeptide of the invention is characterized by one or more of the motifs shown in Table 1.

TABLE 1
CLASP-4 ITAM Motifs

| Motif No. | Sequence Motif |
|---|---|
| 1 | YXXL-X₃-YXXI |
| 2 | YXXI-X₁₃-YXXP |
| 3 | YXX(I/L)-X₃-YXX(I/L)-X₁₃-YXX(I/L) |

The presence of multiple ITAM motifs in CLASP proteins indicates that they may be engaged by multiple signal transduction components (e.g., ZAP-70/Syk, Shc, P13 kinase, and Grb2). In general, the ITAM motif in CLASP proteins match identically to the canonical ITAM motif with some motifs containing a conservative amino acid change (i.e. valine instead of isoleucine or leucine). As previously described for other ITAMs, the ITAMs within CLASPs can bind SH2-containing proteins including ZAP-70, Syk, Shc, PI3 kinase, and Grb2. Since CLASPs have an extracellular domain, CLASPs protein can independently initiate a signal transduction cascade through engagement of its extracellular domain. Otherwise CLASPs may cooperate with an antigen receptor signaling complex (e.g., with CD3/TCR, BCR, FcR), to facilitate tyrosine kinase signal transduction The ITAMs have demonstrated different binding specificity and affinities for SH2 domains (Clements, et al.., 1999, Ann. Rev. Immunol. 17: 89-108). For example, Shc, P13 kinase, and Grb2 bind to dual and mono phosphorylated ITAMs with different affinities. Thus the ITAMs in CLASPs are believed to provide quantitative as well as qualitative differences in signal transduction depending up their phosphorylation state, as well as to inhibit or augment specific protein interactions and hence specific tyrosine kinase-mediated signaling pathways in leukocytes.

Antagonizing the PTK-CLASP-4 interaction (e.g., phosphorylation of CLASP-4) will thus inhibit immune function. In one embodiment, interactions between ITAM-bearing human CLASPs and their binding partners are believed to be antagonized by the alpha subtype (SIRPalpha) of signal regulatory proteins that has been shown to negatively regulate ITAM-dependent lymphocyte activation (Lienard H; 1999, J Biol Chem 274: 32493-9). Also, a recently recognized family of immunoreceptor tyrosine-based inhibition motif (ITIM) receptors are thought to inhibit the ITAM-induced activation of immune competent cells (Gergely, et al., 1999, J. Immunol Lett 68: 3-15) and therefore may block CLASP-partner interaction.

### DOCK

CLASP-4 polypeptides contain a new “DOCK” motif, not previously described in the scientific literature. The CLASP DOCK motif includes a series of five tyrosines surrounded by conserved sequences in regions A, B, C, D, and G (see FIG. 3B). There are also two highly conserved non-tyrosine containing regions (E and G) separated by nine amino acids (P+EXAI+XM) and (LXMXL+GXVXXXVNXG) (where X is any amino acid).

The cytoplasmic region of CLASP-4 immediately following the ITAM domains exhibits sequence similarity to the C-terminal third of the so-called “DOCK” proteins. The DOCK gene family includes three molecules that are the human homologues of the C. elegans CED proteins known to be involved in apoptosis. CED-5 (DOCK180), a major CRK-binding protein, alters cell morphology upon translocation to the membrane ( mediates the membrane motion that scavenger cells exhibit as they surround and engulf dying cells; its function can be partially rescued by the human DOCK180 (Wu et al., 1998, Nature 392: 501-504). Myoblast City in Drosophila (MBC) is another member of the DOCK protein family and has been found to be involved in myoblast fusion (Erickson, et al., 1997, J. Cell Biol. 138: 589). Since CLASP-4 expression is found in syncytial tissues such as placenta, muscle, and heart, it is believed that CLASP-4 is involved in mediating or inhibiting cell fusion.

The DOCK family has been implicated in the control of cell shape. DOCK1, when transfected into spindle cells, can make them flattened and polygonal (Takai, et al., 1996, Genomics 35: 403-303). DOCK1 expression is ubiquitous except in hematopoetic cells. DOCK2 is expressed in hematopoetic cells and when transfected into spindle cells can make them round up (Nishihara, H., 1999, Hokkaido Igaku Zasshi 74: 157-66). DOCK2 is expressed in peripheral blood lymphocytes, thymus, spleen, and liver.

### COILED-COIL

CLASP-4s have the two coiled-coil domains (Lupas et al., 1991, Science 252: 1162-64; Lupas, A., 1996, Meth. Enzymology 266: 513-525). Coiled-coil domains are known to interact directly with cytoskeleton, indicating that that CLASP-4 proteins interact directly with the cytoskeleton. Thus, it is believed that CLASP-4 binds cytoskeletal proteins, e.g., spectrin, ankyrin, hsp70, talin, ezrin, tropomyosin, myosin, plectin, syndecans, paralemmin, Band 3 protein, Cytoskeletal protein 4.1, Tyrosine phosphatase PTP36 and other molecules.

### PBM

Some CLASP proteins comprise a PDZ-binding motif (“PBM” or “PL”) at the C-terminus of the protein. This short (3-8 amino acid) motif mediates the binding of proteins terminating at their carboxyl terminus in the motif (most commonly S/T-X-V-free carboxyl-terminus) to other proteins containing one or more specific PDZ domains (See Songyang et al., 1997, Science 275: 72 and Doyle et al., 1996, Cell 85: 1067 for a discussion of PDZ-ligand structures).

PDZ domain-containing proteins are involved in the organization of ion channels and receptors at the neurological synapse and in establishing and maintaining polarity in epithelial cells via their binding to the C-termini of transmembrane receptors. It has been shown that PDZ-domain containing proteins can mediate protein-protein interactions in immune system cells (e.g., DLG1 binds to the lymphocyte potassium channel KV1.3 in human T lymphocytes, (Hanada et al., 1997, J. Biol. Chem. 272: 26899).

### Modulation of Immune Responses

CLASP-4 proteins, as described above, modulate immune function in a variety of ways and through a variety of mechanisms (i.e., changing the threshold for T cell activation) by affecting formation of the immunological synapse. Establishment and maintenance of the immunological synapse can involve: (A) signal transduction, (B) cell-cell interactions, and (C) membrane organization.

### (A) Signal transduction

Human CLASP proteins, as discussed above, contain SH3 domains and tyrosine phosphorylation sites. These regions have been shown to be involved in signal transduction in a variety of cells including lymphocytes. Thus, human CLASP proteins are believed to interact with these regions during signal transduction events which lead to modulation of immune responses.

CLASP proteins can interact with Tec sub-family of nonreceptor tyrosine kinases. The Tec sub-family of nonreceptor tyrosine kinases consists of Tec, Btk, Tsk/Itk/Emt Itk, and Bmx, and is defined by the presence of SH3 and SH2 domains adjacent to the catalytic domain and an amino-terminal region containing a pleckstrin homology (PH) domain, a Tec homology (TH) domain, and a proline-rich region (Mano, H.; 1999, Cytokine Growth Factor Rev 10: 267-80). The T cell specific Tsk/Itk/Emt, and Btk expressed in most hematopoietic cells other than T cells are important components of antigen receptor signaling pathways in hematopoietic cells.

Btk has been identified as the gene defective in murine X-linked immunodeficiency (xid) and human X-linked agammaglobulinemia (XLA) (Nisitani, S., 2000, Proc Natl Acad Sci U.S.A. 97: 2737-42). In xid mice, B cell numbers are reduced to one-half of normal and the titers of specific immunoglobulin isotypes are significantly reduced; in addition, xid B cells are insensitive to a number of mitogenic stimuli. The human disorder is much more severe, resulting in nearly complete elimination of the B cell compartment and dramatically reduced immunoglobulin levels. Biochemical studies have supported multiple roles for Btk in B cell activation. Btk kinase activity and tyrosine phosphorylation are increased after cross-linking either the B cell receptor on B cells or the high affinity IgE receptor, FcRI, on mast cells. Interleukin-5 and interleukin-6 treatment have also been shown to lead to the activation of Btk.

Itk, like Btk, is tyrosine-phosphorylated upon antigen receptor cross-linking (Mano, H., 1999, Cytokine Growth Factor Rev, 10: 267-80). In addition, peripheral T cells from mice lacking functional Itk are refractory to stimulation by antibodies to CD3 plus antigen presenting cells. These Itk-deficient T cells can be stimulated by phorbol ester and calcium ionophore, demonstrating that Itk acts in signaling pathways proximal to the TCR.

Unlike the related Src family tyrosine kinases including Lyn, Lck, Fyn, ZAP-70, SyK, and CSK, the Tec family kinases lack the amino-terminal myristylation site crucial for the membrane localization of Src family kinases, suggesting that some adaptor proteins are required for the their membrane localization (Mano, H., 1999, Cytokine Growth Factor Rev 10: 267-80). Since all the Tec family kinases contain a proline-rich region which could be bound by a SH3 domain, and since all the human CLASPs contain a SH3 domain, it is believed that human CLASPs could serve as adaptors for the members in the Tec family in different hematopoietic cells.

GTP-binding proteins play an important role in immune response (Mach, B., 1999, Science 285: 1367). A number of biochemical events triggered by TCR/CD3-induced T cell activation are ablated by agents that modulate the action of G proteins. Pertinent to this is the ability of cholera toxin to inhibit the cellular proliferation and intracellular Ca2+ mobilization that is mediated by anti-CD3 antibody treatment of T cells. The G protein competitive inhibitor GDPS, can impede the extent of inositol phosphates generated upon stimulation in peripheral T lymphocytes. Nonhydrolyzable analogs of GTP, such as GTPS, or other agents such as ALF that activate G proteins by circumventing the need for receptor engagement, can result in T cell activation.

The Gaq/11subfamily (Stanners, J., 1995, J Biol Chem 270: 30635-42) and Rapl (Lafont, V., 1998, Biochem Pharmacol 55: 319-24) of GTP-binding proteins have been shown to be involved in human T cell receptor/CD3-mediated signal transduction pathway. Also, Cdc42, a Rho family small GTPase, is known to play a critical role in the formation of actin microspikes in response to external stimuli (Miki, H.; 1998, Nature, 391: 93-6). Interestingly, a Cdc42 binding protein, WASP, has a proline-rich domain which could interact with the SH3 domain present in all the human CLASPs. Human CLASPs may interact with these GTP-binding proteins.

Several adaptor proteins including NCK, CBL (Bachmaier, K., 2000 Nature 403: 211-6), SHC, LNK, SLP-76, HS1, SIT, VAV, GrB2, and BRDG1, and two tyrosine phosphotases, EZRIN, SHP-1 and SHP-2 have been shown to interact with ITAM or SH3 domains. These proteins may also interact with CLASP-4. Several proteins have been shown to interact with ITAM or SH3 domains and may also interact with CLASP-4. These include adaptor proteins such as NCK, CBL (Bachmaier, K., 2000, Nature 403: 211-6), SHC, LAT, LNK, SLP-76 (Krause M et al., 2000, J Cell Biol 149: 181-94), HS1, SIT, VAV, GrB2 (Zhang W. and Samelson, L. E., 2000, Semin Immunol 12: 35-41), and BRDG1, kinases such as SYK and LCK, and tyrosine phosphatases such as SHP-1 and SHP-2. These interactions can be defined by a number of different biochemical or cell biological methods including in vitro binding assays, co-immunoprecipitation assays, co-immunostaining (Harlow, E. and Lane, D., 1999, Using Antibodies: A laboratory Manual. Cold Spring Harbor Press) or genetic assays such as yeast the yeast two hybrid system, in which a CLASP-4 protein or fragment can be used as “bait” (Zervos et al., 1993, Cell 72: 223-232; Madura et al., 1993, J. Biol. Chem 268: 12046-12054).

Other assays include in vitro binding assays, co-immunoprecipitation assays, co-immunostaining assays, and yeast two hybrid system screening assays in which a CLASP-4 domain or fragment can be used as “bait” or “trap” protein (Zervos et al. (1993), Cell 72: 223-232; Madura et al. (1993) J. Biol. Chem. 268: 12046-12054).

In other embodiments, CLASP polypeptides are transfected into lymphocytes. After transfection, a variety of standard assays can be used to evaluate, for example, CLASP modulation of T cell activation. These assays include calcium influx assays, NF-AT nuclear translocation assays (e.g., Cell, 1998, 93: 851-61), NF-AT/luciferase reporter assays (e.g., MCB 1996 16: 7151-7160), tyrosine phosphorylation of early response proteins such as HS1, PLC-γ, ZAP-76, and Vav (e.g., J. Biol. Chem. 1997, 272: 14562-14570).

### (B) Cell-Cell Interaction

As discussed above, human CLASP proteins are homologues of E-cadherin. As shown in FIG. 1, CLASP-4 contains a cadherin ectodomain. Therefore CLASP-4 proteins may interact with cadherins through this domain. The cadherins constitute a family of cell surface adhesion molecules that are involved in calcium-dependent cell to cell adhesion. Human cadherins, E-, P- N- and VE-cadherin, have a restricted tissue distribution: E- and P-cadherin are expressed in epithelial tissues, N-cadherin is found mainly on neural cells, and VE-cadherin is found on vascular endothelium. Homophilic binding between cadherins on adjacent cells is vital for the maintenance of strong cell to cell adhesion in these tissues. For example E-cadherin is required for the formation of adherens junctions between mature epithelial cells and is involved in Langerhans cell adhesion to keratinocytes, and VE-cadherin is needed for the maintenance of lateral association between endothelial cells. The extracellular regions of mature mammalian cadherins are comprised of five “CAD” modules of approximately 1110 amino acids. Crystallographic and biochemical studies indicate that cadherins can form dimers on the cell surface, and that interaction with dimeric cadherins on opposing cell surfaces can lead to the formation of “zipper-like” cell junctions.

The integrins are a second family of transmembrane adhesion molecules that are involved in both cell to cell and cell to matrix interactions. At least 15 chains associate with 8 chains to form a large number of heterodimeric integrins that can be classified into several major subfamilies based on their shared use of a particular chain. Members of three subfamilies, the 1, 2, and 7 integrins, are commonly found on leukocytes. The expression of 1 integrins is widespread (for example, 51, CD49e/CD29, is found on T cells, granulocytes, platelets, fibroblasts, endothelium, and epithelium), whereas the 2 and 7 integrins have a restricted pattern of expression.

Interestingly, E-cadherin on human epithelial cells has been found to be a ligand for the mucosal lymphocyte integrin, E7, and a similar interaction has been indicated in the mouse. Monoclonal antibodies to E-cadherin or to E7 block IEL adherence to epithelial cells, and transfection of cells with E7 confers upon them the ability to adhere to cells transfected with E-cadherin.

L929 cells can be transfected with CLASP-4 and Neomycin. G418-resistant clones can be screened for CLASP-expression with anti-CLASP peptide-specific antibodies. CLASP-expressing clones can be used to test for homotypic and/or heterotypic calcium dependent cell adhesion using the “cell aggregation assay” described for cadherin molecules (Murphy-Erdosh, C. et al., 1995, J. Cell Biol. 129: 1379-1390).

Several approaches can be used to identify the amino acids involved in the binding domains. Soluble fusion molecules (e.g., EC12-IgG, ECC-IgG, ECM-IgG, and GST-EC12), peptides, and peptide-specific anti-CLASP antibodies are available for blocking experiments in the above-described assay. Transfectants generated by site-directed mutagenesis can also be used.

### (C) Membrane Anchoring/Cytoskeletal Interactions

Interestingly, tyrosine-phosphorylated ITAMs interact with actin cytoskeleton upon activation of mature T lymphocytes (Rozdzial, M. M., 1995, Immunity 3: 623-633). Since human CLASPs contain both ITAMs and coiled-coil domains which have been shown to interact with cytoskeletal proteins, CLASPs are believed to play an important role in modulating cell surface molecule expression by re-organizing cytoskeletal structure.

F-actin microfilament cytoskeletal organization has been known to be involved in the modulation of cell surface molecule expression. WASP, a GTPase-binding protein, plays a critical role in the formation of actin microspikes in response to external stimuli and ectopic expression of WASP induces the formation of F-actin filament clusters that overlap with the expressed WASP itself. Another WASP family protein, N-WASP, has also been shown to play important roles in filopodium formation. Both of these proteins cause actin polymerization, but with different features when they are expressed in cells; WASP mainly localizes at perinuclear areas and causes actin clustering, but most N-WASP is present at plasma membranes and induces filopodium formation (Miki, H.; 1998, Nature 391: 93-6). Both WASP and N-WASP, contain a proline-rich domain which could interact with the SH3 domain present in all the human CLASPs. CLASP-4 may interact with F-actin filament through CLASP-4 binding to WASP or WASP-like proteins.

Standard assays can be used for detecting CLASP protein interaction with cytoskeletal proteins. These assays include co-sedimentation assays, far western blot analysis (Ohba, T., 1998, Anal. Biochem. 262: 185-192), surface pasman resonance, F-actin staining with phalloidin in CLASP-transfected lymphocytes (e.g., Small, J. et al. 1999, Microsc. Res. Tech. 4: 3-17), and immunocytal analysis of subcellular distribution of focal adhesion proteins (such as paxillin, tensin, vinculin, talin, and FAK in CLASP-transfected lymphocytes; see, e.g., Ridyard, M.S., 1998, Biochem. Cell Biol. 76: 45-58).

### CLASP-4 Exon Structure and Genomic Domains

Alternative splicing is likely to represent a regulatory switch that governs different functions of CLASP-2 in immune responses Additionally, alternative splice variants affecting the untransilated regions of an RNA can be a way of regulating RNA stability.

As noted supra, CLASP-4 gene expression is characterized by alternative exon usage. Intron/exon structure can be predicted by computer analysis of genomic DNA, however, splice junctions and alternative splicing can only be elucidated by comparison of genomic clones to cDNA clones. Alternative splicing and RNA editing are mechanisms generate a variety of proteins from the same gene. An example for how alternative splicing is used to generate thousands of different proteins from only a few genes is represented by the Neurexin gene family (for review of Neurexins, see Missler M. and Suedhof, T., 1998, Trends in Genetics, 14: 20-25). Comparative analysis of CLASP-4 genomic clones and cDNA clones revealed that CLASP-4 is composed of numerous exons and that distinct CLASP-4 transcripts are generated by alternative splicing.

Numerous diseases are caused or are thought to be caused by splice site mutations that can cause exon skipping or otherwise result in a truncated protein product Some of these diseases include, e.g., Marfan Syndrome (Liu W, et al., 1997, Nat. Genet. 16: 328-9), Hunter disease (Bonucelli G, et al., 2000, Hum. Mutat. (Online) 2000 15(4): 389, Duchenne muscular dystrophy (Wibawa T, et al., 2000, Brain Dev. 22(2): 107-112), Myelomonocytic leukemia (Wutz D, et al., 1999, Leuk. Lymphoma 35: 491-9.), and Isovaleric acidemia (Vockley J, et al., 2000, Am. J. Hum. Genet. 66: 356-67). This is especially true for genes composed of many exons (such as CLASP-4). The genomic sequence around CLASP-4 exon/intron boundaries is useful for diagnostic approaches towards the identification of diseases caused by splice site mutations. The abundance or presence of CLASP-4 isoforms in cell populations (e.g., hematopoietic cells, lymphocytes) may be correlated with a disease state by comparing the abundance of CLASP-4 in cells from subjects suffering from the disease with the level of CLASP-4 in cells from healthy subjects. This can be accomplished by utilizing any number of assays (e.g., PCR). In some embodiments, CLASP introns are included in “minigenes” for improved expression of the CLASP proteins in eukaryotic cells.

A CLASP-4 alternative transcript has been found that inserts 98 nucleotides between nucleotides 3951 and 3952 of FIG. 5 that results in a premature stop codon. The protein produced from this transcript could be a soluble form of CLASP-4. This soluble form of CLASP-2 could act as a natural antagonist or agonist of CLASP-2 function. Further, the soluble form of CLASP-2 could similarly affect the function of other CLASP family members since there is a high degree of amino acid sequence similarity among the CLASP family. Finally, the soluble form of CLASP-2 could have a unique function.

Additionally, a BAC encoding an intronless copy of human CLASP-4 was identified that is thought to be an unexpressed pseudogene. Sequence of this BAC genomic region using primers encoded by the nCLASP-4 cDNA sequence revealed no intron/exon boundaries; however, several point mutations were observed in the genomic region. Some of these point mutations resulted in nonsense codons in the first 100 amino acides of the cDNA sequence, indicating that any protein encoded by this copy of the gene would not be expressed as a polypeptide containing the major functional regions of CLASP family members. It may also produce a soluble, truncated form of CLASP-4 if translated.

### CLASP Superfamily Members

As is illustrated in FIG. 3, CLASP-3 is a member of a superfamily of immune-cell associated proteins with similar motifs (e.g., CLASP-1, 2/6, 3, 4, 5, 7). CLASP-1 is described in WO 00/20434. CLASP-1 uniquely among the known CLASPs contains SH3 binding domain motifs. CLASP-2 is described in WO 00/61747. CLASP-2 polypeptides have no adaptor binding sites or SH3 binding domains found in CLASP-1. Other CLASP family members are described in Application Nos ______; ______; ______ [Attorney Docket Nos. 020054-000311US, 020054-000511US, 020054-000611US] (all filed Dec. 13, 2000), 60/240,508, 60/240,503, 60/240,539, and 60/240,543 (all filed Oct. 13, 2000). The aforementioned publications and applications are all incorporated by reference herein in its entirely for all purposes.

### CLASP-4 mRNA Expression

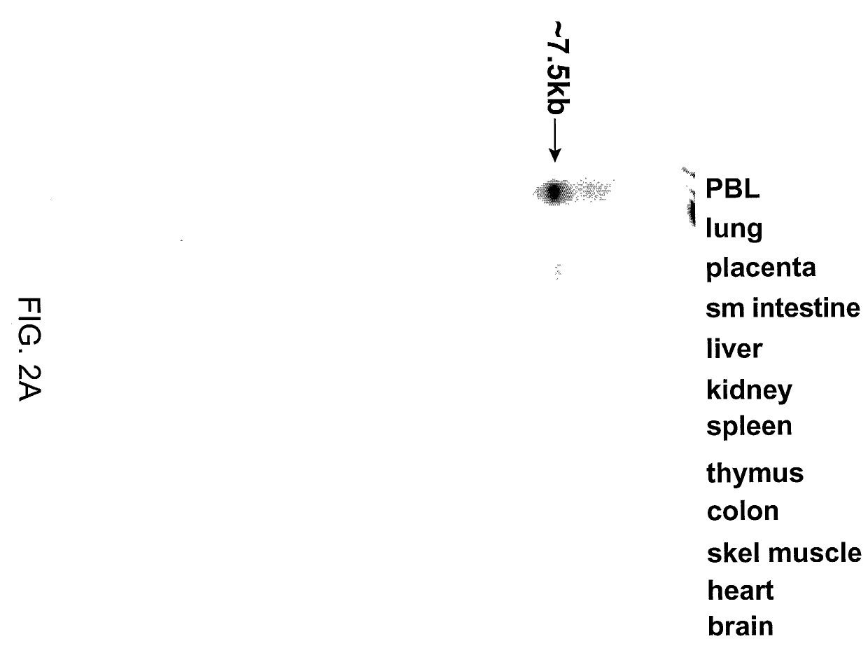

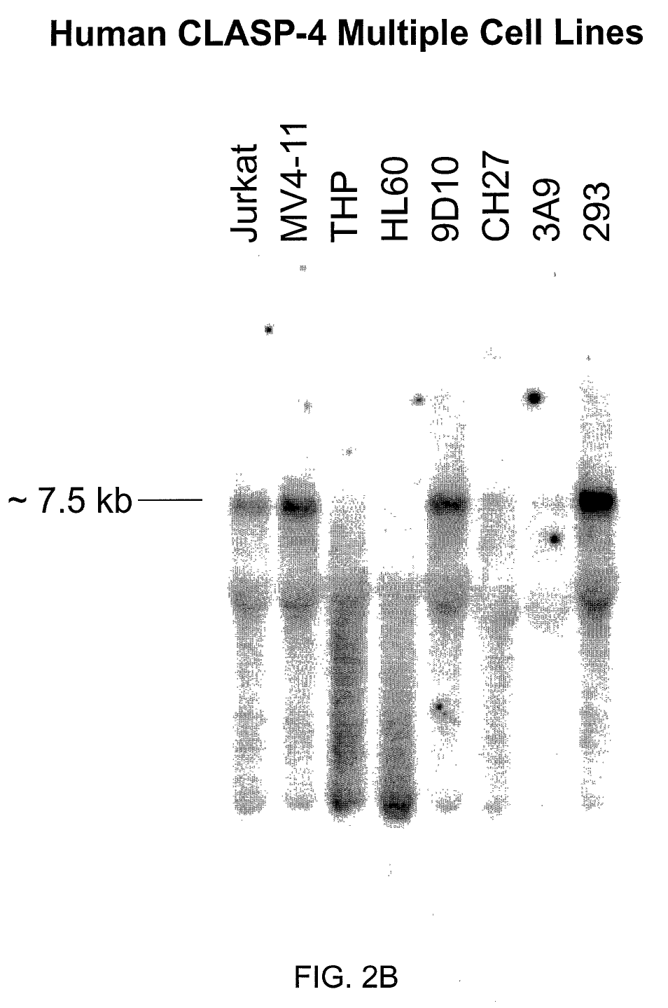

As described in Example 5, CLASP-4 mRNA expression was assayed in tissues and cell lines by Northern analysis. The results are shown in FIG. 2A and B. The results of Northern Analysis of CLASP-4 expression and expression of other members of the CLASP family are summarized in Table 2.

TABLE 2

| | CLASP | | | | | |
|---|---|---|---|---|---|---|
| Tissue/Cell Line¹ | 1 | 2^{3,4} | 3 | 4 | 5 | 7 |
| PBL | +² | − | − | +++ | ++ | − |
| Lung | − | + | − | − | −/+ | +++ |
| Placenta | −/+ | +++ | + | −/+ | + | + |
| Sm Intestine | −/+ | − | − | − | −/+ | + |
| Liver | −/+ | −/+ | −/+ | − | −/+ | + |
| Kidney | −/+ | + | +++ | −/+ | + | ++ |
| Spleen | ++ | − | − | −/+ | + | −/+ |
| Thymus | ++ | − | − | −/+ | + | − |
| Colon | − | − | − | − | − | − |
| Skel Muscle | − | −/+ | ++ | − | − | −/+ |
| Heart | −/+ | ++ | +++ | −/+ | − | +++ |
| Brain | +++ | −/+ | −/+ | − | − | − |
| Jurkat | ++ | ++ | ++ | + | − | − |
| MV411 | ++ | − | ++ | + | + | + |
| THP1 | ++ | − | − | − | − | −/+ |
| HL60 | − | − | − | − | −/+ | − |
| 9D10 | ++ | ++⁵ | + | + | + | + |
| 3A9 | + | −/+ | − | − | − | − |
| CH27 | + | −/+ | − | − | − | − |
| 293 | − | ++ | +++ | + | − | + |

¹Jurkat = human T cell line; MV4-11 = B myelomonocyte; 9D10 = B cell line; THP-1 = monocyte; 3A9 = mouse T cell; CH27 = mouse B cell line; HL60 = human promyclocyte; 293 = embryonic kidney epithelial cells (293)
²Table Legend (based on Northern blot results): − = no expression; −/+ = low expression; + = medium expression; ++ = medium high expression; +++ = high expression.
³A CLASP-2 EST (EST 815795) was identified from a bone marrow cDNA library.
⁴The probe used (HC4.3) encompasses nucleotides 2987 to 3660 from CLASP-4 cDNA sequence as shown in FIG. 1 (nucleotides 4956 to 5629 from the CLASP-4 cDNA sequence as shown in FIG. 5).

As indicated in Table 2 and shown in FIG. 2, CLASP-2 is expressed most strongly in placenta followed by lung, kidney and heart; CLASP-3 is expressed strongly in kidney and heart, and less strongly in placenta and skeletal muscle; CLASP-4 is expressed exclusively in peripheral blood lymphocytes; CLASP-5 is expressed strongly in peripheral blood leukocytes, present in placenta, kidney, spleen and thymus, and weakly in lung, small intestine and liver. It is not expressed in brain, heart, skeletal muscle and large intestine; CLASP-7 is expressed strongly in lung, heart, liver and kidney, but not in PBL, brain or thymus.

Differences in tissue expression patterns for different CLASP proteins indicate different CLASPs have differential roles in immune function and, accordingly, can be separately targeted to achieve different functions. For example, since CLASP proteins are necessary for proper function or signaling by the T cell receptor (TCR), the tissue specific distribution of different CLASPs permits differential modulation of the immune response in different tissues. Since CLASP-2 is present in heart, blocking CLASP-2 function or expression is useful to selectively block immune response in the heart (for example, to selectively stop immune response in the heart compartment, e.g., following cardiac transplant rejection or post-MI inflammation, without compromising immunity elsewhere. Similarly, blocking CLASP-3 can block rejection of the kidney following kidney transplant. Furthermore, by adjusting the level of inhibition, the degree of immune blockage versus response can be modulated in the compartments represented by each CLASP.

### CLASP-4 Polynucleotides And Methods Of Use

The present invention provides a variety of CLASP-4 polynucleotides and methods for using them. In one aspect, the polynucleotide of the invention encodes a polypeptide comprising at least a fragment (e.g., an immunogenic fragment) of a CLASP-4 protein (e.g., at least a fragment of SEQ. ID. NO:2) or variant thereof. In another aspect, the molecules that comprise a CLASP-4 polynucleotide that, while not necessarily encoding a CLASP-4 protein or fragment, is useful as a probe or primer for detecting CLASP-4 expression, for inhibition of CLASP-4 expression (e.g., antisense or ribozyme-mediated inhibition), for gene knockout, and the like.

### CLASP-4 Polynucleotides

The invention also provides isolated or purified nucleic acids having at least 8 nucleotides (i.e., a hybridizable portion) of a CLASP-4 sequence or its complement; in other embodiments, the nucleic acids consist of at least about 25 (continuous) nucleotides, about 50 nucleotides, about 100 nucleotides, about 150 nucleotides, about 200 nucleotides, about 250 nucleotides, about 500 nucleotides, about 550 nucleotides, about 600 nucleotides, or about 650 nucleotides or more of a CLASP-4 sequence, or a full-length CLASP-4 coding sequence. In another embodiment, the nucleic acids are smaller than about 35, about 200 or about 500 nucleotides in length. Polynucleotides can be single or double stranded, and may be DNA, RNA, PNA or a hybrid molecule.

In specific aspects, nucleic acids are provided which comprise a sequence complementary to at least about 10, 25, 50, 100, 150, 200, 250, 500, 550, 600, or 650 nucleotides or the entire coding region of a CLASP-4 coding sequence. Usually, the isolated polynucleotide is less than about 100 kbp, generally less than about 50 kbp, and often less than about 20 kbp, less than about 10 kbp, less than about 5 kbp, or less than about 1000 nucleotides in length.

In a specific embodiment, a nucleic acid that is hybridizable to a CLASP-4 nucleic acid or its complement, or to a nucleic acid encoding a CLASP-4 derivative, under conditions of low stringency is provided. Derivatives of CLASP-4 contemplated include, but are not limited to, splice variants of a gene encoding a CLASP-4, other members of a CLASP-4 gene family which differ from one of the CLASP-4 nucleotide or amino acid sequences disclosed herein by the insertion or deletion of one or several domains, and the like.

In one embodiment, the CLASP-4 polynucleotide is identical or exactly complementary to SEQ. ID NO: 1 or selectively hybridizes to an aforementioned sequence. In various embodiments, the polynucleotide is identical or exactly complementary to, or selectively hybridizes to, the nucleotide sequence encoding a particular protein domain or region, or a particular gene exon of the CLASP-4 mRNA or genomic sequence. Such polynucleotides are particularly useful as probes, because they can be selected to identify a defined species of CLASP-4.

In addition to the polypeptide and polynucleotide sequences specifically exemplified herein, the invention contemplates CLASP-4 homologues from other species, allelic and splice variants, and other variants disclosed herein.

### Substantial Identity

In some embodiments, the CLASP-4 polynucleotides of the invention are substantially identical to SEQ ID NOs: 1 or to a fragment thereof.

An indication that two nucleic acid sequences are substantially identical is that the two polynucleotides have a specified percentage sequence identity e.g., usually at least about 75%, at least about 80%, at least about 85%, at least about 90%, at least about 95%, or at least about 98 identity over a specified region when optimally aligned.

Another indication that two nucleic acid sequences are substantially identical is that a polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

Yet another indication that two nucleic acid sequences are substantially identical (e.g., a naturally occurring allele of the CLASP-4 sequence of SEQ ID NO:1) is that the same primers can be used to amplify the sequence. For example, CLASP-4 polynucleotides can be PCR amplified from cDNA derived from human lymphocytes using the primer pairs shown in Table 3.

The primers of Table 3 are also useful for amplification of CLASP-4 splice variants. Another indication that two nucleic acid sequences are substantially identical is that they selective hybridize under stringent conditions (i.e., one sequence hybridizes to the complement of the second sequence), as described infra.

### Selective Hybridization

The invention also relates to nucleic acids that selectively hybridize to exemplified CLASP-4 sequences (including hybridizing to the exact complements of these sequences). Selective hybridization can occur under conditions of high stringency (also called “stringent hybridization conditions”), moderate stringency, or low stringency.

### High Stringency

“Stringent hybridization conditions” are conditions under which a probe will hybridize to its target subsequence, typically in a complex mixture of nucleic acid, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5× SSC and 1% SDS incubated at 42° C. or 5× SSC and 1% SDS incubated at 65° C., with a wash in 0.2× SSC and 0.1% SDS at 65° C. In a specific embodiment, a nucleic acid which is hybridizable to a CLASP-4 nucleic acid under the following conditions of high stringency is provided: Prehybridization of filters containing DNA is carried out for 8 h to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 8-16 h at 65° C. in prehybridization mixture containing 100 μg/ml denatured salmon sperm DNA and 5-20×10⁶ cpm of ³²P-labeled probe. Washing of filters is done at 65° C. for 15-30 h in a solution containing 2× SSC, 0.1% SDS. This is followed by a wash in 0.2× SSC and 0.1% at 50° C. for 15-30 min before autoradiography.

### Moderate Stringency

In another specific embodiment, a nucleic acid, which is hybridizable to a CLASP-4 nucleic acid under conditions of moderate stringency is provided. Examples of procedures using such conditions of moderate stringency are as follows: Filters containing DNA are pretreated for 6 h at 55° C. in a solution containing 6× SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 12-16 h at 55° C., and then washed twice for 30 minutes at 50° C. in a solution containing 1× SSC and 0.1% SDS. Filters are blotted dry and exposed for autoradiography. Other conditions of moderate stringency which can be used are well-known in the art. Washing of filters is done at 45° C. for 1 h in a solution containing 0.2× SSC and 0.1% SDS.

### Low Stringency

By way of example and not limitation, procedures using such conditions of low stringency are as follows (see also Shilo and Weinberg, 1981, Proc. Natl. Acad. Sci. U.S.A. 78: 6789-6792): Filters containing DNA are pretreated for 6 h at 40° C. in a solution containing 35% formamide, 5× SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 g/ml salmon sperm DNA, 10% (wt/vol) dextran sulfate, and 5-20×106 cpm 32P-labeled probe is used. Filters are incubated in hybridization mixture for 18-20 h at 40° C., and then washed for 1.5 h at 55° C. in a solution containing 2× SSC and 0.1% SDS. The wash solution is replaced with fresh solution and incubated an additional 30 minutes at 50-55° C. Filters are blotted dry and exposed for autoradiography. If necessary, filters are washed for a third time at 60-65° C. and reexposed to film. Other conditions of low stringency that can be used are well known in the art (e.g., as employed for cross-species hybridizations).

### CLASP-4 Variants and Fragments

The CLASP-4 variants of the invention can contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. CLASP-4 polynucleotide variants can be produced for a variety of reasons, e.g., to optimize codon expression for a particular host (change codons in the human mRNA to those preferred by a bacterial host such as E. coli).

Exemplary CLASP-4 polynucleotide fragments are preferably at least about 15 nucleotides, and more preferably at least about 20 nucleotides, still more preferably at least about 30 nucleotides, and even more preferably, at least about 40 nucleotides in length, or larger, e.g., at least about 50, 150, 200, 250, 300, 350, 400, 450, 500, 550, 600, 650 nucleotides. Exemplary fragments include fragments having at least a sequence from about nucleotide number 1-50, 51-100, 101-150, 151-200, 201-250, 251-300, 301-350, 351-400, 401-450, 451-500, 501-550, 551-600 to the end of the CLASP-3 polynucleotide sequence shown in FIG. 5 or comprising the cDNA coding sequence in a deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In one embodiment, the CLASP-4 variants differ from SEQ ID NO:1 by virtue of incorporating a different combination of exons than found in the exemplified sequences.

Using known methods of protein engineering and recombinant DNA technology, variants can be generated to improve or alter the characteristics of the CLASP-4 polypeptides. For instance, one or more amino acids can be deleted from the N-terminus or C-terminus of the CLASP-4 protein without substantial loss of biological function.

Furthermore, even if deleting one or more amino acids from the N-terminus or C-terminus of a polypeptide results in modification or loss of one or more biological functions, other biological activities can still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking Nor C-terminal residues of a protein retains such immunogenic activities can readily be determined by routine methods described herein and otherwise known in the art.

Thus, the invention further includes CLASP-4 polypeptide variants which show biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247: 1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at 30 specific positions of a cloned gene to identify regions critical for protein function. For example., site directed mutagenesis or alanine-scanning mutagenesis (introduction of single alanine mutations at every residue in the molecule) can be used. (Cunningham and Wells, 1989, Science 244: 1081-1085) The resulting mutant molecules can then be tested for biological activity.

In various embodiments, CLASP-4 polynucleotide fragments include coding regions for, or regions hybridizable to, the CLASP-4 structural or functional domains described supra. As set out in the Figures, such preferred regions include the following domains/motifs: ITAM, DOCK, COILED/COILED, and PBM. Thus, for example, polypeptide fragments of SEQ ID NO:2 falling within conserved domains are specifically contemplated by the present invention (see FIG. 3). Moreover, polynucleotide fragments encoding these domains are also contemplated. Such polypeptide fragments find use, for example, as inhibitors of CLASP-4 function in CLASP-4-expressing cells.

### Uses of CLASP-4 Polynucleotides

The CLASP-4 polynucleotides of the invention are useful in a variety of applications. In one aspect of the invention, the polypeptide-encoding CLASP-4 polynucleotides of the invention are used to express CLASP-4 polypeptides (e.g., as described herein) for example to produce anti-CLASP-antibodies or for use as therapeutic polypeptides. In another aspect, the CLASP-4 polynucleotide or fragments thereof can be used for diagnostic purposes (e.g., as probes for CLASP-4 expression). In particular, since CLASP-4s can be expressed in lymphocytes, a CLASP-4 polynucleotide can be used to detect the expression of CLASP-4 as a lymphocyte marker. For diagnostic purposes, a CLASP-4 polynucleotide can be used to detect CLASP-4 gene expression or aberrant CLASP-4 gene expression in disease states. In another aspect, the CLASP-4 polynucleotide or fragments are used for therapeutic purposes. For example, included in the scope of the invention are methods for inhibiting CLASP-4 expression, e.g., using oligonucleotide sequences, such as antisense RNA and DNA molecules and ribozymes, that function to inhibit expression of CLASP-4. In another aspect, CLASP-4 polynucleotides can be used to construct transgenic and knockout animals, e.g., for screening of CLASP-4 agonists and antagonists. In another aspect, CLASP-4 polynucleotides can be used for screening of CLASP-4 agonists and antagonists.

### Uses of CLASP-3 Promoter Sequence

A variety of uses of the CLASP promoter sequence provided herein will be apparent to one of skill reviewing this disclosure. In an embodiment, reporter genes are operably linked to CLASP upstream sequences containing promoter elements. The resulting vectors have numerous uses, including identification of cis and trans transcriptional regulatory factors in vivo and for screening of agents capable of modulating (e.g., activating or inhibiting) CLASP expression (e.g., drug screening). In an embdoiment, for example, a modulator of CLASP expression can be identified by detecting the effect of the modulator on expression of a reporter gene whose expression is regulated, in whole or part, by a naturally occurring CLASP regulatory element (e.g., promoter or enhancer). A number of reporters may be used (e.g., firefly luciferase, β-glucuronidase, β-galactosidase, chloramphenicol acetyl transferase, SEAP, GFP). In a related embodiment, a CLASP coding sequence is used in place of a reporter and changes in CLASP protein expression (or activity) is detected using the methods disclosed herein. In a related embodiment, the ability of a test compound to bind to a CLASP gene regulatory sequence is assayed.

Changes in CLASP activity or expression can be measured by any suitable method (e.g., monitoring levels of CLASP gene products (e.g., protein and RNAs) by hybridization immunoassays, RNAse protection assays, amplification assays, or any other suitable detection means described herein or known in the art. Quantitating amounts of nucleic acid in a sample (e.g., evaluating levels of RNA) is also useful in evaluating cis- or trans- transcriptional regulators. Assay formats for identification of compounds that affect expression and activity of proteins are well known in the biotechnological and pharmaceutical industries, and numerous additional assays and variations of the illustrative assays provided herein will be apparent to those of skill. The promoter sequences of the invention can also be used in the preparation of gene “knock-out vectors” discussed herein.

### Use of CLASP-4 Polynucleotides for Detection, Diagnosis, and Treatment

The CLASP-4 polynucleotides of the invention are useful for detection of CLASP-4 expression in cells and in the diagnosis of diseases or disorders (e.g., immunodeficient states) resulting from aberrant expression of CLASP-4. Aberrant expression of CLASP-4 mRNA or protein means expression in lymphocytes (e.g., T lymphocytes or B lymphocytes) or other CLASP-4 expressing cells of at least 2-fold, preferably at least 5-fold greater or less than expression in control lymphocytes obtained from a healthy subject. CLASP-4 polypeptide expression is easily measured by ELISA using anti-CLASP-4 antibodies of the invention. CLASP-4 mRNA expression (including expression of specific species or splice variants of CLASP-4) can be measured by quantitative Northern analysis or quantitative PCR, LCR, or other methods, using the probes and primers of the invention.

In one embodiment, the assays of the present invention are amplification-based assays for detection of an CLASP-4 gene product. In an amplification based assay, all or part of a CLASP-4 mRNA or cDNA (hereinafter also referred to as “target”) is amplified, and the amplification product is then detected directly or indirectly. When there is no underlying gene product to act as a template, no amplification product is produced (e.g., of the expected size), or amplification is non-specific and typically there is no single amplification product. In contrast, when the underlying gene or gene product is present, the target sequence is amplified, providing an indication of the presence and/or quantity of the underlying gene or mRNA. Target amplification-based assays are well known to those of skill in the art.

The present invention provides a wide variety of primers and probes for detecting CLASP-4 genes and gene products. Such primers and probes are sufficiently complementary to the CLASP-4 gene or gene product to hybridize to the target nucleic acid. Primers are typically at least 6 bases in length, usually between about 10 and about 100 bases, typically between about 12 and about 50 bases, and often between about 14 and about 25 bases in length, often PCR primers of 15-30 (e.g., 18-22 nucleotides) are used. However, the length of primers can be adjusted by one skilled in the art. One of skill, having reviewed the present disclosure, will be able, using routine methods, to select primers to amplify all, or any portion, of the CLASP-4 gene or gene product, or to distinguish between variant gene products, CLASP-4 alleles, and the like. Single oligomers (e.g., U.S. Pat. No. 5,545,522), nested sets of oligomers, or even a degenerate pool of oligomers can be employed for amplification.

It will be appreciated that probes and primers can be selected to distinguish between species and splice variants based on the guidance of this disclosure, by targeting primers or probes to differentially used exons (or exon-exon junctions that differ between variants).

Methods can include the steps of collecting a sample of cells from a patient, isolating nucleic acid (e.g., genomic, mRNA or both) from the cells of the sample, contacting the nucleic acid sample with one or more primers which specifically hybridize to an CLASP-4 gene under conditions such that hybridization and amplification of the CLASP-4-gene (if present) occurs, and detecting the presence or absence of an amplification product, or detecting the size of the amplification product and comparing the length to a control sample. See U.S. Pat. Nos. 4,683,195 and 4,683,202, Landegran et al., 1988, Science 241: 1077-1080; Nakazawa et al., 1994, Proc. Natl. Acad. Sci. U.S.A. 91: 360-364, Abravaya et al., 1995, Nucleic Acids Res. 23: 675-682).

Because CLASP-4 gene products are expressed in the immune system (e.g., T lymphocytes, B lymphocytes and macrophages), expression will be typically assayed in these cells. Methods which are well known to those skilled in the art can be used to isolate lymphocytes, macrophages, and alike (See, e.g., Coligan, J. E., et al. (eds.), 1991, Current Protocols in Immunology, John Wiley & Sons, N.Y.; this reference is incorporated by reference for all purposes). In one embodiment, assays are carried out on biopsy or autopsy-derived tissue.

In various embodiments, CLASP-4 gene expression is detected by hybridization of a detectable probe to mRNA or cDNA obtained from cells (e.g., lymphocytes). A variety of methods for specific DNA and RNA measurement using nucleic acid hybridization techniques are known to those of skill in the art (see Sambrook et al., supra). Hybridization based assays refer to assays in which a probe nucleic acid is hybridized to a target nucleic acid, forming a hybridization complex. Usually the nucleic acid hybridization probes of the invention are entirely or substantially identical to a contiguous sequence of the CLASP-4 gene or RNA sequence. Preferably, nucleic acid probes are at least about 50 bases, often at least about 20 bases, and sometimes at least about 200 bases, at least about 300-500 nucleotides or more in length. Various hybridization techniques are well known in the art, and are in fact the basis of many commercially available diagnostic kits.

Methods of selecting nucleic acid probe sequences for use in nucleic acid hybridization are discussed in Sambrook et al., supra. In some formats, at least one of the target and probe is immobilized. The immobilized nucleic acid can be DNA, RNA, or another oligo- or poly-nucleotide, and can comprise natural or non-naturally occurring nucleotides, nucleotide analogs, or backbones. Such assays can be in any of several formats including: Southern, Northern, dot and slot blots, high-density polynucleotide or oligonucleotide arrays (e.g., GeneChips™ Affymetrix), dip sticks, pins, chips, or beads. All of these techniques are well known in the art and are the basis of many commercially available diagnostic kits. Hybridization techniques are generally described in Hames et al., ed., 1985, Nucleic Acid Hybridization, A Practical Approach IRL Press; Gall and Pardue, 1969, Proc. Natl. Acad. Sci. U.S.A., 63: 378-383; and John et al., 1969, Nature, 223: 582-587.

A variety of nucleic acid hybridization formats are known to those skilled in the art. For example, one common format is direct hybridization, in which a target nucleic acid is hybridized to a labeled, complementary probe. Typically, labeled nucleic acids are used for hybridization, with the label providing the detectable signal. One method for evaluating the presence, absence, or quantity of CLASP-4 mRNA is carrying out a Northern transfer of RNA from a sample and hybridization of a labeled CLASP-4 specific nucleic acid probe A useful method for evaluating the presence, absence, or quantity of DNA encoding CLASP-4 proteins in a sample involves a Southern transfer of DNA from a sample and hybridization of a labeled CLASP-4 specific nucleic acid probe.

Other common hybridization formats include sandwich assays and competition or displacement assays. Sandwich assays are commercially useful hybridization assays for detecting or isolating nucleic acid sequences. Such assays utilize a “capture” nucleic acid covalently immobilized to a solid support and a labeled “signal” nucleic acid in solution. The biological or clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

In one embodiment, CLASP-4 polypeptides or polynucleotides are useful in treating deficiencies or disorders of the immune system, by activating or inhibiting the activation, differentiation of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders can be genetic, somatic, such as cancer or some autoimmune disorders, acquired (e.g., by chemotherapy or toxins), or infectious.

In another embodiment, CLASP-4 polynucleotides or polypeptides are useful in treating or detecting deficiencies or disorders of hematopoietic cells. CLASP-4 polypeptides or polynucleotides could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g., agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

In one embodiment, CLASP-4 polynucleotides or polypeptides are useful in treating or detecting autoimmune diseases. The term “autoimmune disease” as used herein has the normal meaning in the art and refers to a spontaneous or induced malfunction of the immune system of mammals in which the immune system fails to distinguish between foreign immunogenic substances within the mammal and/or autologous (“self”) substances and, as a result, treats autologous (“self”) tissues and substances as if they were foreign and mounts an immune response against them. Autoimmune disease is characterized by production of either antibodies that react with self tissue, and/or the activation of immune effector T cells that are autoreactive to endogenous self antigens. Three main immunopathologic mechanisms act to mediate autoimmune diseases: 1) autoantibodies are directed against functional cellular receptors or other cell surface molecules, and either stimulate or inhibit specialized cellular function with or without destruction of cells or tissues; 2) autoantigen--autoantibody immune complexes form in intercellular fluids or in the general circulation and ultimately mediate tissue damage; and 3) lymphocytes produce tissue lesions by release of cytokines or by attracting other destructive inflammatory cell types to the lesions. These inflammatory cells in turn lead to production of lipid mediators and cytokines with associated inflammatory disease.

Since many autoimmune disorders result from inappropriate recognition of self as foreign material by immune cells. This inappropriate recognition results in an immune response leading to the destruction of the host tissue. Therefore, the administration of CLASP-4 polypeptides or polynucleotides that can inhibit an immune response, particularly the proliferation, or differentiation of T-cells, can be an effective therapy in preventing autoimmune disorders.

Examples of autoimmune disorders that can be treated or detected by CLASP-4 include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, can also be treated by CLASP-4 polypeptides or polynucleotides. Moreover, CLASP-4 can be used to treat anaphylaxis or hypersensitivity to an antigenic molecules.

In one embodiment CLASP-4 polynucleotides or polypeptides are used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of CLASP-4 polypeptides or polynucleotides that inhibits an immune response, particularly the proliferation, differentiation of T-cells, can be an effective therapy in preventing organ rejection or GVHD.

Similarly, in another embodiment, CLASP-4 polypeptides or polynucleotides are used to modulate inflammation. The term “inflammation” refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils.

For example, CLASP-4 polypeptides or polynucleotides can inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.). Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

In another embodiment CLASP-4 polypeptides or polynucleotides are used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. CLASP-4 polypeptides or polynucleotides can be used to treat or detect any of these symptoms or diseases.

### Use of CLASP-4 Polynucleotides in Screening

The presence or absence of hCLASP-4 nucleotide and amino acid sequences in a biological sample can be used in screening assays as medical diagnostics to aid in clinical decision-making. As discussed above, hCLASP-4 is expressed at high levels in peripheral blood leukocytes (PBMCs). Therefore, in one embodiment, hCLASP-4-based diagnostics involves screening assays for the detection hCLASP-4 nucleotide and amino acid sequences in PBMCs. Detection can be achieved by standard assays known to one of skill in the art including quantitative RT-PCR, Northern analysis, Western analysis, flow cytometry/fluorescence-activated cell sorting (FACS), ELISA, immunoflourescence and immunoperoxidase staining using anti- hCLASP-4 antibodies (Sambrook, Fritsch and Maniatas, 1989, Molecular Cloning, 2nd Ed, Cold Spring Harbor Lab. Press; Harlow et. al. ,1988, Antibodies, a laboratory manual, Cold Spring Harbor Lab. Press).

In another embodiment, hCLASP-4-based diagnostics involves screening assays for the diagnosis of infections of the urinary system. In another embodiment, hCLASP-4-based diagnostics involves screening assays to determine the presence or absence of leukocyte esterase in urine (Fauci et al Eds., Harrison's Principles of Internal Medicine, 14th Ed. McGraw Hill, 1998, pp. 817-22). Detection of the presence or absence of elevated numbers of leukocytes in urine is useful for the diagnosis of infections of the urinary system. Current diagnostics routinely test urine for the presence of leukocyte esterase (Fauci et al., (eds.), Harrison's Principles of Internal Medicine, 14th Ed. McGraw Hill, 1998, pp. 817-22). Detection of hCLASP-4 would provide a viable alternative to current methods.

In another embodiment, hCLASP-4-based diagnostics involves screening assays for the detection of elevated numbers of leukocytes in cerebrospinal fluid for the diagnosis of meningitis and similar disorders. In another embodiment, hCLASP-4-based diagnostics involves screening assays for detecting autoimmune disorders of the central nervous system (e.g., multiple sclerosis) (Fauci et al Eds., Harrison's Principles of Internal Medicine, 14th Ed. McGraw Hill, 1998, pp. 2409-39). Detection of elevated levels of hCLASP-4 nucleic acid or amino acid sequence in cerebrospinal fluid can indicate the presence of these disorders. Synovial fluid, which contains elevated numbers of leukocytes during joint infection and autoimmune arthritis (e.g. rheumatoid arthritis or systemic lupus erythematosis) could also be screened using hCLASP-4-based diagnostics.

In another embodiment, hCLASP-4-based diagnostics involve screening assays for identifying disorders of cells of hematopoietic lineage. hCLASP-4 is expressed in human T cells, B cell lines and in myelomonocytic cells but not monocytic or myelocytic cells. In the French-American-British (FAB) classification of acute myelogenous leukemias, M4 (acute myelomonocytic leukemia) has a subset (M4eo) that is associated with excellent prognosis (Cotran, et al., 1999, Robbins Pathologic Basis of Disease, 6th edition, Saunders). The use of hCLASP-4 may help to further subdivide M4 and permit a better forecast of prognosis. Precise identification of hematopoietic cell types is vital to guide chemotherapy and radiation therapy therapy of patients with leukemia and lymphoma (Fauci et al., (eds.), 1998, Harrison's Principles of Internal Medicine, 14th Ed., McGraw Hill, pp. 695-712). hCLASP-4 expression differences can be detected, for example, by using FACS, immunofluorescence, immunoperoxidase staining, RT-PCR, in situ hybridization or RNA blot analysis (Sambrook, Fritsch and Maniatas, 1989, Molecular Cloning, 2nd Ed., Cold Spring Harbor Lab. Press; Ward MS, 1999, Pathology 31(4): 382-92).

In another embodiment, hCLASP-4-based diagnostics involve screening assays for identifying activated immune system cells by FACS. Although hCLASP-4 is generally expressed at high levels in PBMCs, it is known that the surface expression of the closely related mouse CLASP-1 protein is altered during the process of lymphocyte activation. An analogous change in expression is expected for the hCLASP-4 protein. Subtyping lymphocytes specific for a particular antigen, for example, using MHC-based multimeric staining reagents (Altman et. al., 1996, Science 274: 94-6), for separating cell populations into hCLASP-4 high and hCLASP-4 low populations, can aid in determining the nature of the immune response against that antigen. Such understanding is critical, for example, in predicting the course of chronic viral infections such as hepatitis B, hepatitis C, and HIV, and to designing appropriate treatment regimens for patients suffering from these infections.

### CLASP-4 Antisense, Ribozyme and Triplex Polynucleotides and Methods of Use

Oligonucleotide sequences, that include anti-sense RNA and DNA molecules and ribozymes that function to inhibit the translation of a CLASP-4 mRNA are within the scope of the invention. Such molecules are useful in cases where downregulation of CLASP-4 expression is desired. Anti-sense RNA and DNA molecules act to directly block the translation of mRNA by binding to targeted mRNA and preventing protein translation. The invention provides methods and antisense oligonucleotide or polynucleotide reagents which can be used to reduce expression of CLASP-4 gene products in vitro or in vivo. Administration of the antisense reagents of the invention to a target cell results in reduced CLASP activity. As will be apparent to one of skill and as discussed supra (Table 3), specific CLASP-4 splice variants can be specifically targeted for inhibition. Alternatively, by designing an, e.g., antisense molecule that recognizes a sequence found in several or all CLASP-4 species, a general inhibition can be achieved.

### A. Antisense

Without intending to be limited to any particular mechanism, it is believed that antisense oligonucleotides bind to, and interfere with the translation of, the sense CLASP-4 mRNA. Alternatively, the antisense molecule can render the CLASP-4 mRNA susceptible to nuclease digestion, interfere with transcription, interfere with processing, localization or otherwise with RNA precursors (“pre-mRNA”), repress transcription of mRNA from the CLASP-4 gene, or act through some other mechanism. However, the particular mechanism by which the antisense molecule reduces CLASP-4 expression is not critical.

The antisense polynucleotides of the invention comprise an antisense sequence of at least 7 to 10 to typically 20 or more nucleotides that specifically hybridize to a sequence from mRNA encoding CLASP-4 or mRNA transcribed from the CLASP-4 gene. More often, the antisense polynucleotide of the invention is from about 10 to about 50 nucleotides in length or from about 14 to about 35 nucleotides in length. In other embodiments, antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors. Generally, to assure specific hybridization, the antisense sequence is substantially complementary to the target CLASP-4 mRNA sequence. In certain embodiments, the antisense sequence is exactly complementary to the target sequence. The antisense polynucleotides can also include, however, nucleotide substitutions, additions, deletions, transitions, transpositions, or modifications, or other nucleic acid sequences or non-nucleic acid moieties so long as specific binding to the relevant target sequence corresponding to CLASP-4 RNA or its gene is retained as a functional property of the polynucleotide.

It will be appreciated that the CLASP-4 polynucleotides and oligonucleotides of the invention can be made using nonstandard bases (e.g., other than adenine, cytidine, guanine, thymine, and uridine) or nonstandard backbone structures to provides desirable properties (e.g., increased nuclease-resistance, tighter-binding, stability or a desired TM). Techniques for rendering oligonucleotides nuclease-resistant include those described in PCT publication WO 94/12633. A wide variety of useful modified oligonucleotides may be produced, including oligonucleotides having a peptide-nucleic acid (PNA) backbone (Nielsen et al., 1991, Science 254: 1497) or incorporating 2′-O-methyl ribonucleotides, phosphorothioate nucleotides, methyl phosphonate nucleotides, phosphotriester nucleotides, phosphorothioate nucleotides, phosphoramidates. Still other useful oligonucleotides may contain alkyl and halogen-substituted sugar moieties comprising one of the following at the 2′ position: OH, SH, SCH3, F, OCN, OCH3OCH3, OCH3O(CH2)nCH3, O(CH2)nNH2 or O(CH2)nCH3, where n is from 1 to about 10; C1 to C10 lower alkyl, substituted lower alkyl, alkaryl or aralkyl; Cl; Br; CN; CF3; OCF3; O-, S-, or N-alkyl; O-, S-, or N-alkenyl; SOCH3 ; SO2CH3; ONO2; NO2; N3; NH2; heterocycloalkyl; heterocycloalkaryl; aminoalkylamino; polyalkylamino; substituted silyl; an RNA cleaving group; a cholesteryl group; a folate group; a reporter group; an intercalator; a group for improving the pharmacokinetic properties of an oligonucleotide; or a group for improving the pharmacodynamic properties of an oligonucleotide and other substituents having similar properties. Folate, cholesterol or other groups that facilitate oligonucleotide uptake, such as lipid analogs, may be conjugated directly or via a linker at the 2′ position of any nucleoside or at the 3′ or 5′=0 position of the 3′-terminal or 5′-terminal nucleoside, respectively. One or more such conjugates may be used. Oligonucleotides may also have sugar mimetics such as cyclobutyls in place of the pentofuranosyl group. Other embodiments may include at least one modified base form or “universal base” such as inosine, or inclusion of other nonstandard bases such as queosine and wybutosine as well as acetyl-, methyl-, thio- and similarly modified forms of adenine, cytidine, guanine, thymine, and uridine which are not as easily recognized by endogenous endonucleases. The antisense oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The invention further provides oligonucleotides having backbone analogues such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, chiral-methyl phosphonates, nucleotides with short chain alkyl or cycloalkyl intersugar linkages, short chain heteroatomic or heterocyclic intersugar (“backbone”) linkages, or CH2-NH-O-CH2, CH2-N(CH3)-OCH2, CH2-O-N(CH3)-CH2, CH2-N(CH3)-N(CH3)-CH2 and O-N(CH3)-CH2-CH2 backbones (where phosphodiester is O-P-O-CH2), or mixtures of the same. Also useful are oligonucleotides having morpholino backbone structures (U.S. Pat. No. 5,034,506).

Useful references include Oligonucleotides and Analogues, A Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan et al., Jul. 9, 1993, J. Med. Chem. 36(14): 1923-1937; Antisense Research and Applications (1993, CRC Press), in its entirety and specifically Chapter 15, by Sanghvi, entitled “Heterocyclic base modifications in nucleic acids and their applications in antisense oligonucleotides;” and Antisense Therapeutics, ed. Sudhir Agrawal (Humana Press, Totowa, N.J., 1996).

In one embodiment, the antisense sequence is complementary to relatively accessible sequences of the CLASP-4 mRNA (e.g., relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner et al., 1997, Nature Biotechnology 15: 537). Examples of oligonucleotides that can be tested in cells for antisense suppression of CLASP-4 function are those capable of hybridizing to (i.e., substantially complementary to) CLASP-4 at the following positions:

| Oligo | Sequence 5′-3′ | length | notes/comments |
|---|---|---|---|
| 1 | TGGGAACATAAGCAG | 32-mer | spans nucleotides −23 |
| | ATCTCGGAGGGGGTCG | | to +9 of the sequence |
| | C | | of FIG. 5, and covers |
| | | | the putative |
| | | | initiator methionine |
| 2 | GGGAAATCTCTGGCCT | 31-mer | spans nucleotides |
| | CGGGGAAGCTTAATC | | 2748 to 2778 of the |
| | | | sequence of FIG.5, |
| | | | and is complementary |
| | | | to the region |
| | | | encoding the cadherin |
| | | | cleavage site |
| 3 | CTAGAGCTGCTACATG | 31-mer | spans nucleotides |
| | GACATAACACATCGC | | 4720 to 4750 of the |
| | | | sequence of FIG.5, |
| | | | and |
| | | | is complementary to |
| | | | the region encoding |
| | | | the transmembrane |
| | | | domain |

In some embodiments, administration of antisense oligonucleotides can result in reduction of hCLASP-mRNA expression by at least about 50%, as assessed by Northern analysis after administration of an antisense phosphorothioate oligonucleotide at a concentration of 1 μM, 5 μM, 10 μM or 20 μM.

The invention also provides an antisense polynucleotide that has sequences in addition to the antisense sequence (i.e., in addition to anti-CLASP-4-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention can be prepared by de novo chemical synthesis or by cloning. For example, an antisense RNA that hybridizes to CLASP-4 mRNA can be made by inserting (ligating) an CLASP-4 DNA sequence (e.g., SEQUENCE ID No: 1, or fragment thereof) in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention. The term “operably linked” refers to a functional linkage between a nucleic acid expression control sequence (such as a promoter or enhancer) and a second nucleic acid sequence, wherein the expression control sequence directs transcription of the nucleic acid corresponding to the second sequence.

In one embodiment, antisense DNA oligodeoxyribonucleotides derived from the translation initiation site, e.g., between −10 and +10 regions of a CLASP-4 nucleotide sequence, are used. For general methods relating to antisense polynucleotides, see ANTISENSE RNA AND DNA, 1988, D. A. Melton, Ed., Cold Spring Harbor Laboratory, Cold Spring Harbor, N.Y.). See also, Dagle et al., 1991, Nucleic Acids Research, 19: 1805. For a review of antisense therapy, see, e.g., Uhlmann et al., 1990, Chem. Reviews, 90: 543-584.

### B. Ribozyme

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of CLASP-4 RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features such as secondary structure that can render the oligo-nucleotide sequence unsuitable. The suitability of candidate targets can also be evaluated by testing their accessibility to hybridization with complemen-tary oligonucleotides, using ribonuclease protection assays.

### C. Triplex

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6): 569-584; Helene et al., 1992, Ann. N.Y. Acad. Sci., 660: 27-36; and Maher, 1992, Bioassays 14(12): 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleotide sequences can be pyrimidine-based, which will result in TAT and CGC+triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

### D. General

The anti-sense RNA and DNA molecules, ribozymes and triple helix molecules of the invention can be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules can be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

Methods for introducing polynucleotides into such cells or tissue include methods for in vitro introduction of polynucleotides such as the insertion of naked polynucleotide, i e., by injection into tissue, the introduction of a CLASP-4 polynucleotide in a cell ex vivo, the use of a vector such as a virus, (e.g., a retrovirus, adenovirus, adeno-associated virus, and the like), phage or plasmid, and the like or techniques such as electroporation or calcium phosphate precipitation.

### Gene Therapy

By introducing gene sequences into cells, gene therapy can be used to treat conditions in which the cells do not express normal CLASP-4 or express abnormal/inactive CLASP-4. In some instances, the polynucleotide encoding a CLASP-4 is intended to replace or act in the place of a functionally deficient endogenous gene. Alternatively, abnormal conditions characterized by overexpression can be treated using the gene therapy techniques described below.

In a specific embodiment, nucleic acids comprising a sequence encoding a CLASP-4 protein or functional derivative thereof, are administered to promote CLASP-4 function, by way of gene therapy. Gene therapy refers to therapy performed by the administration of a nucleic acid to a subject. In this embodiment of the invention, the nucleic acid produces its encoded protein that mediates a therapeutic effect by promoting CLASP-4 function.

Any of the methods for gene therapy available in the art can be used according to the present invention. Exemplary methods are described below.

For general reviews of the methods of gene therapy, see, Goldspiel et al., 1993, Clinical Pharmacy 12: 488-505; Wu and Wu, 1991, Biotherapy 3: 87-95; Tolstoshev, 1993, Ann. Rev. Pharmacol. Toxicol. 32: 573-596; Mulligan, 1993, Science 260: 926-932; and Morgan and Anderson, 1993, Ann. Rev. Biochem. 62: 191-217; Can, 1993, TIBTECH 11(5): 155-215). Methods commonly known in the art of recombinant DNA technology which can be used are described in Ausubel et al., supra; and Kriegler, 1990, Gene Transfer and Expression, A Laboratory Manual, Stockton Press, N.Y.

In one aspect, the therapeutic composition comprises a CLASP-4 nucleic acid that is part of an expression vector that encodes a CLASP-4 protein or fragment or chimeric protein thereof in a suitable host. In particular, such a nucleic acid has a promoter operably linked to the CLASP-4 coding region, said promoter being inducible or constitutive, and, optionally, tissue-specific. In another particular embodiment, a nucleic acid molecule is used in which the CLASP-4 coding sequences and any other desired sequences are flanked by regions that promote homologous recombination at a desired site in the genome, thus providing for intrachromosomal expression of the CLASP-4 nucleic acid (Koller and Smithies, 1989, Proc. Natl. Acad. Sci. U.S.A. 86: 8932-8935; Zijlstra et al., 1989, Nature 342: 435-438).

Delivery of the nucleic acid into a patient can be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see, e.g., Wu and Wu, 1987, J. Biol. Chem. 262: 4429-4432) (which can be used to target cell types specifically expressing the receptors), and the like. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO 92/20316 dated Nov. 26, 1992; WO 93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. U.S.A. 86: 8932-8935; Zijlstra et al., 1989, Nature 342: 435-438).

In a specific embodiment, a viral vector that contains the CLASP-4 nucleic acid is used. For example, a retroviral vector can be used (see, Miller et al., 1993, Meth. Enzymol. 217: 581-599). These retroviral vectors have been modified to delete retroviral sequences that are not necessary for packaging of the viral genome and integration into host cell DNA. The CLASP-4 nucleic acid to be used in gene therapy is cloned into the vector, which facilitates delivery of the gene into a patient. More detail about retroviral vectors can be found in Boesen et al., 1994, Biotherapy 6: 291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., 1994, J. Clin. Invest. 93: 644-651; Kiem et al., 1994, Blood 83: 1467-1473; Salmons and Gunzberg, 1993, Human Gene Therapy 4: 129-141; and Grossman and Wilson, 1993, Curr. Opin. in Genetics and Devel. 3: 110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson 1993, Current Opinion in Genetics and Development 3: 499-503) present a review of adenovirus-based gene therapy. Bout et al., 1994, Human Gene Therapy 5: 3-10, demonstrated the use of adenovirus vectors to transfer genes to the respiratory epithelia of rhesus monkeys. Other instances of the use of adenoviruses in gene therapy can be found in Rosenfeld et al., 199 1, Science 252: 431-434; Rosenfeld et al., 1992, Cell 68: 143-155; and Mastrangeli et al., 1993, J. Clin. Invest. 91: 225-234. Adeno-associated virus (AAV) has also been proposed for use in gene therapy (Walsh et al., 1993, Proc. Soc. Exp. Biol. Med. 204: 289-300.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, and the like. Numerous techniques are known in the art for the introduction of foreign genes into cells (see, e.g., Loeffler and Behr, 1993, Meth. Enzymol. 217: 599-618; Cohen et al., 1993, Meth. Enzymol. 217: 618-644; Cline, 1985, Pharmac. Ther. 29: 69-92) and can be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells can be applied as a skin graft onto the patient. Recombinant blood cells (e.g., hematopoietic stem or progenitor cells) are preferably administered intravenously. The amount of cells envisioned for use depends on the desired effect, patient state, and the like., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and the like. In a preferred embodiment, the cell used for gene therapy is autologous to the patient.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### Knockout Cells

In one aspect of the invention, endogenous target gene expression can also be reduced by inactivating or “knocking out” the target gene or its promoter using targeted homologous recombination (see, e.g., Smithies et al., 1985, Nature 317: 230-234; Thomas and Capecchi, 1987, Cell 51: 503-512; Thompson et al., 1989, Cell 5: 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (see, e.g., Thomas and Capecchi, 1987 and Thompson, 1989, supra). However, this approach can be adapted for use in humans provided the recombinant DNA constructs are directly administered or targeted to the required site in vivo using appropriate viral vectors.

### Transgenic and Knockout Animals

The CLASP-4 gene product can also be expressed in transgenic animals. Animals of any species, including, but not limited to, mice, rats, rabbits, guinea pigs, pigs, micro-pigs, goats, sheep, and non-human primates, e.g., baboons, monkeys, and chimpanzees can be used to generate CLASP-4 transgenic animals. The term “transgenic,” as used herein, refers to animals expressing CLASP-4 gene sequences from a different species (e.g., mice expressing human CLASP-4 gene sequences), as well as animals that have been genetically engineered to overexpress endogenous (i.e., same species) CLASP-4 sequences or animals that have been genetically engineered to no longer express endogenous CLASP-4 gene sequences (i.e., “knock-out” animals), and their progeny.

Any technique known in the art can be used to introduce a CLASP-4 transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten et al., 1985, Proc. Natl. Acad. Sci., U.S.A. 82: 6148-6152); gene targeting in embryonic stem cells (Thompson et al., 1989, Cell 56: 313-321); electroporation of embryos (Lo, 1983, Mol. Cell. Biol. 3: 1803-1814); and sperm-mediated gene transfer (Lavitrano et al., 1989, Cell 57: 717-723) (For a review of such techniques, see Gordon, 1989, Transgenic Animals, Intl. Rev. Cytol. 115, 171-229) Any technique known in the art can be used to produce transgenic animal clones containing a CLASP-4 transgene, for example, nuclear transfer into enucleated oocytes of nuclei from cultured embryonic, fetal or adult cells induced to quiescence (Campbell et al., 1996, Nature 380: 64-66; Wilmut et al., Nature 385: 810-813).

The present invention provides for transgenic animals that carry a CLASP-4 transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teaching of Lasko et al. (1992, Proc. Natl. Acad. Sci. U.S.A. 89: 6232-6236). The regulatory sequences required for such a cell-type specific activation will depend upon the particular cell type of interest, and will be apparent to those of skill in the art. When it is desired that the CLASP-4 transgene be integrated into the chromosomal site of the endogenous CLASP-4 gene, gene targeting is preferred. Briefly, when such a technique is to be utilized, vectors containing some nucleotide sequences homologous to the endogenous CLASP-4 gene are designed for the purpose of integrating, via homologous recombination with chromosomal sequences, into and disrupting the function of the nucleotide sequence of the endogenous CLASP-4 gene. The transgene can also be selectively introduced into a particular cell type, thus inactivating the endogenous CLASP-4 gene in only that cell type, by following, for example, the teaching of Gu et al. (1994, Science 265: 103-106). The regulatory sequences required for such a cell-type specific inactivation will depend upon the particular cell type of interest, and will be apparent to those of skill in the art.

Once transgenic animals have been generated, the expression of the recombinant CLASP-4 gene can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques that include, but are not limited to, Northern blot analysis of tissue samples obtained from the animal, in situ hybridization analysis, and RT-PCR (reverse transcriptase PCR). Samples of CLASP-4 gene-expressing tissue, can also be evaluated immunocytochemically using antibodies specific for the CLASP-4 transgene product.

### Other Uses of CLASP-4 Polynucleotides

There exists an ongoing need to identify new chromosome marking reagents. Sequences can be mapped to chromosomes by preparing PCR primers from SEQ ID NO: 1. These primers can be can be less than 50 nucleotides in length, generally less than 46 nucleotides, more generally less than 41 nucleotides, most generally less than 36 nucleotides, preferably less than 31 nucleotides, more preferably less than 26 nucleotides, and most preferably less than 21 nucleotides in length. The probes can also be less than 16 nucleotides, less than 13 nucleotides in length, less than 9 nucleotides in length and less than 7 nucleotides in length. Primers can be selected so that the primers do not span more than one predicted exon in the genomic DNA. These primers are then used for PCR screening of somatic cell hybrids containing individual human chromosomes (i.e., chromosome 13). Only those hybrids containing the human CLASP-4 gene corresponding to SEQ ID NO: 1 will yield an amplified fragment.

Similarly, somatic hybrids provide a rapid method of PCR mapping the polynucleotides to particular chromosomes. Precise chromosomal location of the CLASP-4 polynucleotides can also be achieved using fluorescence in situ hybridization (FISH) of a metaphase chromosomal spread. See Verma, et al, Human Chromosomes: A Manual of Basic Techniques, Pergamon Press. N.Y., 1988. Once a polynucleotide has been mapped to an exact chromosomal location, the physical position of the polynucleotide can be used in linkage analysis. Linkage analysis establishes coinheritance between a chromosomal location and presentation of a particular disease. See McKusick, V., 1998, Mendelian Inheritance in Man: A Catalog of Human Genes and Genetic Disorders, 12th Ed, Johns Hopkins University Press.

The CLASP-4 polynucleotides can be used for identifying individuals from minute biological samples as DNA markers for restriction fragment length polymorphism (RFLP). An individual's genomic DNA is digested with one or more restriction enzymes, and probed on a Southern blot with CLASP-4 DNA markers to yield unique bands for identifying the individual

As described above, it has demonstrated that upon sequencing of numerous independent cDNA products, single nucleotide polymorphisms (SNPs) and small deletion possibly due to RNA editing have been discovered within CLASP-4. These alterations and differences are presented in FIG. 5B. The SNPs represent mis-sense alterations. The small deletion of nucleotides 2556-2560 causes a frame shift that leads to a premature stop codon. Protein translated from this transcript could generate a truncated form of CLASP-4 protein that could have unique fuction or act as an agonist or antagonist for CLASP-4 function.

If it is determined that certain SNPs are deleterious or advantageous, SNPs could be used as a diagnostic tool through SNP mapping or direct sequencing of the SNP region to determine which isoform is expressed. Additionally, the SNPs could be used as a general SNP marker for chromosomal defects such as rearrangement and translocations.

CLASP-4 polynucleotides can be also be used as polymorphic markers for forensic analysis. See generally National Research Council, The Evaluation of Forensic DNA Evidence (Eds.), 1996, Pollard et al., National Academy Press, Washington D.C.). The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample. The CLASP-4 polynucleotide sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the CLASP-4 nucleotide sequences or portions thereof, e.g., fragments derived from the noncoding regions of SEQ ID NO:1 having a length of at least 20 bases, preferably at least 25 bases, and more preferably at least 30 bases.

CLASP-4 polynucleotides can also be used as reagents for paternity testing. The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child. Of course, the present invention can be expanded to the use of this procedure to determine if one individual is related to another. Even more broadly, the present invention can be employed to determine how related one individual is to another, for example, between races or species.

Bacterial infections are a major cause of health-related problems. However, the emergence of drug resistant bacteria is compromising the therapeutic value of the present spectrum of antibiotics. All the currently used antibiotics are small organic molecules, with certain level of structural similarity. This provides an advantage for bacteria to develop drug resistance, since they need to modify a limited number of genes in order to become resistant to a wide variety of antibiotics. The development of antibiotics with different chemical structure and targets can overcome antibiotic resistance, and provide therapeutic superiority in preventing infection by bacterial pathogens. Additionally, most antibiotics are not naturally occurring compounds and cause minor or sometimes serious side effects. For example, antibiotics used to treat TB can cause hearing loss.

The present invention provides new antibacterial agents. Certain CLASP-4 DNA sequences were difficult to clone and subclone (see Example 1). Bacteria harboring certain pieces of CLASP cDNA products were unable to be isolated, indicating that introduction of CLASP sequencescompromised bacterial viability. There can be at least two possible reasons why the CLASP cDNA were unable to be cloned, which can reflect a variation of the well-established Modification and Restriction systems found in bacteria (reviewed in Wilson and Murray. (1991) Annu. Rev. Genet. 25:585-627; Bickle and Kruger (1993) Microbiol. Rev. 57:29-67). This well-described system is used by bacteria to prevent deleterious effects caused by the introduction of foreign DNA. Bacteria can recognize foreign DNA since it does not have the same modifications (e.g. methylation) as the native DNA. After recognition, the bacteria then digest and eliminate the foreign DNA (restriction). In the first scenario, the CLASP cDNA can be recognized as foreign DNA, and digested and eliminated as in the Modification and Restriction system. However, this would be unique for CLASP cDNA since the bacteria used for cloning cDNA are compromised in the Modification and Restriction system, which makes cloning of cDNA into bacteria a practice common in the art. If this is the case, the bacterial apparatus that specifically recognizes or eliminates CLASP cDNA can provide a novel target to develop antimicrobial agents. The CLASP DNA sequence would be useful in targeting the apparatus as well as an entry point for designing screens to identify potential targets. The second possibility is that CLASP CDNA behaves as an antimicrobial agent (i.e., antibiotic), and prevents bacterial growth. This, in effect, would create a new type of antibiotic mediated by the presence of foreign DNA (i.e. CLASP cDNA). In the case for the CLASP cDNA, the bacteria can recognize the DNA but instead of digesting and eliminating the DNA, the CLASP cDNA can cause a variation of the restriction and prevent the bacteria from growing, imposing a bacteriacidal effect upon the bacteria.

DNA as an antimicrobial agent has significant advantages over currently available agents. First, it is structurally unrelated to any existing antibiotics, and can overcome the present growing drug-resistance problem to structurally common agents. Second, since DNA antimicrobials composed of naturally-occurring human DNA, are expected to have minimal side effects and immune rejection. Third, DNA sequences can be tailored with sequence variation and numerous chemical modifications to circumvent the problem of resistance. Fourth, the antimicrobial DNA can be delivered specifically to bacterial cells through the use of bacteriophages (i.e., bacterial virus) which specifically infect bacteria and do not infect human cells. Further specificity can be generated to infect certain bacteria and bacterial subpopulations. Finally, this system can be economically robust since the generation of DNA and delivery vehicles are inexpensive.

### Polypeptides Encoded by the CLASP-4 Gene Coding Sequence

In accordance with the invention, a CLASP-4 polynucleotide which encodes the CLASP-4 polypeptides, mutant polypeptides, peptide fragments, CLASP-4 fusion proteins or functional equivalents thereof, can be used to express CLASP-4 proteins in appropriate host cells. In various embodiments, the CLASP-4 polypeptides expressed will be identical or substantially similar to SEQ ID NOs: 2 or a fragment thereof.

In some embodiments, altered DNA sequences which can be used in accordance with the invention include deletions, additions or substitutions of different nucleotide residues resulting in a sequence that encodes the same or a functionally equivalent gene product. For example, due to the inherent degeneracy of the genetic code, other DNA sequences which encode substantially the same or a functionally equivalent amino acid sequence, can be used in the practice of the invention for the expression of the CLASP-4 protein. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. One of skill will recognize that each codon in a nucleic acid sequence such SEQ ID NO:1 (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Thus, for example, due to the degeneracy of the genetic code, a polypeptide having the sequence of SEQ ID NO:2 or a fragment thereof, can be encoded by numerous polynucleotides other than SEQ ID NO:1. Typically, the degenerate sequence will hybridize with SEQ ID NO: 1 under high or moderate stringency conditions, but this is not strictly required (e.g., when a copy of a nucleic acid is created using the maximum codon degeneracy permitted by the genetic code. In such cased, the nucleic acids typically hybridize under moderately stringent hybridization conditions.)

The gene product itself can contain deletions, additions or substitutions of amino acid residues within a CLASP-4 sequence, which result in a silent change thus producing a functionally equivalent CLASP-4 protein. Such conservative amino acid substitutions can be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine, histidine and arginine; amino acids with uncharged polar head groups having similar hydrophilicity values include the following: glycine, asparagine, glutamine, serine, threonine, tyrosine; and amino acids with nonpolar head groups include alanine, valine, isoleucine, leucine, phenylalanine, proline, methionine, tryptophan. Creighton, 1984, PROTEINS, has grouped amino acids that are conservative substitutions for one another as follows: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M).

The DNA sequences of the invention can be engineered in order to alter a CLASP-4 coding sequence for a variety of ends, including but not limited to, alterations which modify processing and expression of the gene product. For example, mutations can be introduced using techniques which are well known in the art, e.g., site-directed mutagenesis, to insert new restriction sites, to alter glycosylation patterns, phosphorylation, and the like. Based on the domain organization of the CLASP-4 proteins, a large number of CLASP-4 mutant polypeptides can be constructed by modifying or rearranging the nucleotide sequences that encode the CLASP-4 extracellular, transmembrane and cytoplasmic domains.

In various embodiments, the present invention provides homologues of the CLASP-4 polypeptides which function as either an CLASP-4 agonists or an CLASP-4 antagonist. In a preferred embodiment, the CLASP-4 agonists and antagonists stimulate or inhibit, respectively, a subset of the biological activities of the naturally occurring form of the CLASP-4 polypeptide. Thus, specific biological effects can be elicited by treatment with a homologue of limited function. In one embodiment, treatment of a subject with a homologue having a subset of the biological activities of the naturally occurring form of the polypeptide has fewer side effects in a subject relative to treatment with the naturally occurring form of the CLASP-4 polypeptide.

The invention contemplates both full-length CLASP-4 polypeptides and fragments, e.g., fragments having a length of at least about 10, often 20, frequently 50 or 100 residues substantially identical to the exemplified CLASP-4 polypeptide sequences of the invention. Protein fragments can be “free-standing,” or comprised within a larger polypeptide of which the fragment forms a part or region, most preferably as a single continuous region. Representative examples of polypeptide fragments of the invention, include, for example, fragments from about amino acid number 1-20, 2 1-40, 4 1-60, 61-80, 81-100, 102-120, 121-140, 141-160, 161-180, 181-200, or 201 to the end of the coding region. Moreover, polypeptide fragments can be about 20, 30, 40, 50, 60, 70, 80, 90, 100, 110, 120, 130, 140, 150, 200 amino acids in length. In this context “about” includes the particularly recited ranges, larger or smaller by several (5, 4, 3, 2, or 1) amino acids, at either extreme or at both extremes.

Preferred polypeptide fragments include the CLASP-4 protein. Further preferred polypeptide fragments include the CLASP-4 protein having a continuous series of deleted residues from the amino or the carboxy terminus, or both. For example, any number of amino acids, ranging from 1-X, can be deleted from the amino terminus of either the CLASP-4 polypeptide. Furthermore, any combination of the above amino and carboxy terminus deletions are preferred. Similarly, polynucleotide fragments encoding these CLASP-4 polypeptide fragments are also preferred.

Even if deletion of one or more amino acids from the N-terminus of a protein results in modification of loss of one or more biological functions of the protein, other biological activities can still be retained. Thus, the ability of shortened CLASP-4 muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a CLASP-4 mutein with a large number of deleted N-terminal amino acid residues can retain some biological or immunogenic activities. In fact, peptides composed of as few as four CLASP-4 amino acid residues can often evoke an immune response.

Homologues of the CLASP-4 polypeptide can be generated by mutagenesis, e.g., discrete point mutation or truncation of the CLASP-4 polypeptide. As used herein, the term “homologue” refers to a variant form of the CLASP-4 polypeptide which acts as an agonist or antagonist of the activity of the CLASP-4 polypeptide. An agonist of the CLASP-4 polypeptide can retain substantially the same, or a subset, of the biological activities of the CLASP-4 polypeptide. An antagonist of the CLASP-4 polypeptide can inhibit one or more of the activities of the naturally occurring form of the CLASP-4 polypeptide, by, for example, competitively binding to a downstream or upstream member of the CLASP-4 molecular pathway which includes the CLASP-4 polypeptide.

Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT, β-galactosidase, β-glucuronidase, GFP (see, e.g., Mistili & Spector, 1997, Nature Biotechnology 15: 961-964); changes in signal transduction, phosphorylation and dephosphorylation, receptor-ligand interactions, second messenger concentrations (e.g., cGMP, cAMP, IP3, and Ca2+), and cell growth. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

### Synthesis or Expression of CLASP-4 Polypeptide Expression Systems

In order to express a biologically active CLASP-4, the nucleotide sequence coding for CLASP-4, or a functional equivalent, is inserted into an appropriate expression vector. The CLASP-4 gene product as well as host cells or cell lines transfected or transformed with recombinant CLASP-4 expression vectors can be used for a variety of purposes. These include, but are not limited to, generating antibodies (i.e., monoclonal or polyclonal) that competitively inhibit activity of CLASP-4 protein and neutralize its activity; antibodies that activate CLASP-4 function and antibodies that detect its presence on the cell surface or in solution. Anti-CLASP-4 antibodies can be used in detecting and quantifying expression of CLASP-4 levels in cells and tissues such as lymphocytes and macrophages, as well as isolating CLASP-4-positive cells from a cell mixture.

Methods which are well known to those skilled in the art can be used to construct recombinant expression vectors containing the CLASP-4 coding sequence and appropriate transcriptional/translational control signals. These methods include in vitro recombinant DNA techniques, synthetic techniques and in vivo recombination/genetic recombination. (See, e.g., the techniques described in Sambrook et al., 1989, Molecular Cloning A Laboratory Manual, Cold Spring Harbor Laboratory, N.Y. and Ausubel et al., supra). The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. It will be appreciated by those skilled in the art that the design of the expression vector can depend on such factors as the choice of the host cell to be transformed, the level of expression of polypeptide desired, and the like. The expression vectors of the invention can be introduced into host cells to thereby produce polypeptides or peptides, including fusion polypeptides or peptides, encoded by nucleic acids as described herein (e.g., CLASP-4 polypeptides, mutant forms of CLASP-4, fusion polypeptides, and the like).

A variety of host-expression vector systems can be utilized to express a CLASP-4 coding sequence. These include, but are not limited to, microorganisms such as bacteria transformed with recombinant bacteriophage DNA, plasmid DNA, or cosmid DNA expression vectors containing the CLASP-4 coding sequence; yeast transformed with recombinant yeast expression vectors containing the CLASP-4 coding sequence; insect cell systems infected with recombinant virus expression vectors (e.g., baculovirus) containing the CLASP-4 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the CLASP-4 coding sequence; or animal cell systems. The expression elements of these systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter; cytomegalovirus promoter) and the like can be used; when cloning in insect cell systems, promoters such as the baculovirus polyhedron promoter can be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) can be used; when cloning in mammalian cell systems, promoters derived from the genome of mammalian cells (e.g., metallothionein promoter) or from mammalian viruses (e.g., the adenovirus late promoter; the vaccinia virus 7.5K promoter) can be used; when generating cell lines that contain multiple copies of the CLASP-4 DNA, SV40-, BPV- and EBV-based vectors can be used with an appropriate selectable marker.

In bacterial systems a number of expression vectors can be advantageously selected depending upon the use intended for the expressed CLASP-4 product. For example, when large quantities of CLASP-4 protein are to be produced for the generation of antibodies or to screen peptide libraries, vectors which direct the expression of high levels of fusion protein products that are readily purified can be desirable. Such vectors include, but are not limited to, the E. coli expression vector pUR278 (Ruther et al., 1983, EMBO J. 2: 1791), in which the CLASP-4 coding sequence can be ligated into the vector in frame with the lacZ coding region so that a hybrid protein is produced; pIN vectors (Inouye & Inouye, 1985, Nucleic acids Res. 13: 3101-3109; Van Heeke & Schuster, 1989, J. Biol. Chem. 264: 5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In yeast, a number of vectors containing constitutive or inducible promoters can be used. (Current Protocols in Molecular Biology, Vol. 2, 1988 (Suppl. 1999), Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II.) In cases where plant expression vectors are used, the expression of the CLASP-4 coding sequence can be driven by any of a number of promoters. For example, viral promoters such as the 35S RNA and 19S RNA promoters of CaMV (Brisson et al., 1984, Nature 310: 511-514), or the coat protein promoter of TMV (Takamatsu et al., 1987, EMBO J. 6: 307-311) can be used; alternatively, plant promoters such as the small subunit of RUBISCO (Coruzzi et al., 1984, EMBO J. 3: 1671-1680; Broglie et al., 1984, Science 224: 838-843); or heat shock promoters, e.g., soybean hspl7.5-E or hspl7.3-B (Gurley et al., 1986, Mol. Cell. Biol. 6: 559-565) can be used. These constructs can be introduced into plant cells using Ti plasmids, Ri plasmids, plant virus vectors, direct DNA transfor-mation, microinjection, electroporation, and the like. (Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.)

An alternative expression system which could be used to express CLASP-4 is an insect system. In one such system, Autographa californica nuclear polyhedrosis virus (AcNPV) is used as a vector to express foreign genes. The virus grows in Spodoptera frugiperda cells. The CLASP-4 coding sequence can be cloned into non-essential regions (e.g., the polyhedron gene) of the virus and placed under control of an AcNPV promoter (e.g., the polyhedron promoter). Successful insertion of the CLASP-4 coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al., 1983, J. Viol. 46: 584; Smith, U.S. Pat. No. 4,215,051).

In mammalian host cells, a number of viral based expression systems can be utilized. In cases where an adenovirus is used as an expression vector, the CLASP-4 coding sequence can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing CLASP-4 in infected hosts. (See, e.g., Logan & Shenk, 1984, Proc. Natl. Acad. Sci. U.S.A. 81: 3655-3659). Alternatively, the vaccinia 7.5K promoter can be used. (See, e.g., Mackett et al., 1982, Proc. Natl. Acad. Sci. U.S.A. 79: 7415-7419; Mackett et al., 1984, J. Virol. 49: 857-864; Panicali et al., 1982, Proc. Natl. Acad. Sci. U.S.A. 79: 4927-4931). Regulatable expression vectors such as the tetracycline repressible vectors can also be used to express a coding sequence in a controlled fashion.

Specific initiation signals can also be required for efficient translation of inserted CLASP-4 coding sequences. These signals include the ATG initiation codon and adjacent sequences. In cases where the entire CLASP-4 gene, including its own initiation codon and adjacent sequences, is inserted into the appropriate expression vector, no additional translational control signals can be needed. However, in cases where only a portion of the CLASP-4 coding sequence is inserted, exogenous translational control signals, including the ATG initiation codon, must be provided. Furthermore, the initiation codon must be in phase with the reading frame of the CLASP-4 coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, and the like. (see Bittner et al., 1987, Methods in Enzymol. 153: 516-544).

In addition, a host cell strain can be chosen which modulates the expression of the inserted sequences, or modifies and processes the gene product in a specific fashion desired. Such modifications (e.g., glycosylation) and processing (e.g., cleavage) of protein products can be important for the function of the protein. The presence of several consensus N-glycosylation sites in CLASP-4 extracellular domains support the possibility that proper modification can play a role in CLASP-4 function. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, W138, and the like.

Host cells transformed with nucleotide sequences encoding CLASP-4 may be cultured under conditions suitable for the expression and recovery of the soluble protein from cell culture. The protein produced by a transformed cell may be secreted or contained intracellularly depending on the sequence and/or the vector used. As will be understood by those of skill in the art, expression vectors containing polynucleotides which encode CLASP-4 may be designed to contain signal sequences which direct secretion of CLASP-4 through a prokaryotic or eukaryotic cell membrane. Other constructions may be used to join sequences encoding CLASP-4 to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin,

For long-term, high-yield production of recombinant proteins, stable expression is preferred. For example, cell lines which stably express CLASP-4 proteins can be engineered. Rather than using expression vectors which contain viral origins of replication, host cells can be transformed with the CLASP-4 DNA controlled by appropriate expression control elements (e.g., promoter, enhancer, sequences, transcription terminators, polyadenylation sites, and the like.), and a selectable marker. Following the introduction of foreign DNA, engineered cells can be allowed to grow for 1-2 days in an enriched medium, and then switched to a selective medium. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the CLASP-4 protein(s) on the cell surface. Such engineered cell lines are particularly useful in screening for molecules or drugs that affect CLASP-4 function.

A number of selection systems can be used, including but not limited to, the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. U.S.A. 48: 2026), and adenine phosphoribosyltransferase (Lowy et al., 1980, Cell 22: 817) genes which can be employed in tk-, hgprt- or aprt- cells, respectively. Also, antimetabolite resistance can be used as the basis of selection for dhfr, which confers resistance to methotrexate (Wigler et al., 1980, Natl. Acad. Sci. U.S.A. 77: 3567; O'Hare et al., 1981, Proc. Natl. Acad. Sci. U.S.A. 78: 1527); gpt, which confers resistance to mycophenolic acid (Mulligan & Berg, 1981), Proc. Natl. Acad. Sci. U.S.A. 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. U.S.A. 85: 8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10: 169).

In an alternate embodiment of the invention, the coding sequence of CLASP-4 could be synthesized in whole or in part, using chemical methods well known in the art. (See, e.g., Caruthers et al., 1980, Nuc. Acids Res. Symp. Ser. 7: 215-233; Crea and Horn, 180, Nuc. Acids Res. 9(10): 2331; Matteucci and Caruthers, 1980, Tetrahedron Letter 21: 719; and Chow and Kempe, 1981, Nuc. Acids Res. 9(12): 2807-2817.) Alternatively, the protein itself could be produced using chemical methods to synthesize a CLASP-4 amino acid sequence in whole or in part. For example, peptides can be synthesized by solid phase techniques, cleaved from the resin, and purified by preparative high perform-ance liquid chromatography. (See Creighton, 1983, Proteins Structures And Molecular Principles, W. H. Freeman and Co., N.Y. pp. 50-60). The composition of the synthetic polypeptides can be confirmed by amino acid analysis or sequencing (e.g., the Edman degradation procedure; see Creighton, 1983, Proteins, Structures and Molecular Principles, W. H. Freeman and Co., N.Y., pp. 34-49).

In some embodiments, the CLASP-4 polypeptide contains non-naturally occurring amino acids or amino acid analogs (i.e., compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium).

### Identification of Cells That Express CLASP-4

The recombinant host cells which contain the coding sequence and which express a CLASP-4 gene product or fragments thereof can be identified by at least four general approaches; (a) DNA-DNA or DNA-RNA hybridization; (b) the presence or absence of “marker” gene functions; (c) assessing the level of transcription as measured by the expression of CLASP-4 mRNA transcripts in the host cell; and (d) detection of the gene product as measured by immunoassay or by its biological activity. Prior to the identification of gene expression, the host cells can be first mutagenized in an effort to increase the level of expression of CLASP-4, especially in cell lines that produce low amounts of CLASP-4.

In the first approach, the presence of the CLASP-4 coding sequence inserted in the expression vector can be detected by DNA-DNA or DNA-RNA hybridization using probes comprising nucleotide sequences that are homologous to the CLASP-4 coding sequence, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, and the like). For example, if the CLASP-4 coding sequence is inserted within a marker gene sequence of the vector, recombinants containing the CLASP-4 coding sequence can be identified by the absence of the marker gene function. Alternatively, a marker gene can be placed in tandem with the CLASP-4 sequence under the control of the same or different promoter used to control the expression of the CLASP-4 coding sequence. Expression of the marker in response to induction or selection indicates expression of the CLASP-4 coding sequence.

In the third approach, transcriptional activity for the CLASP-4 coding region can be assessed by hybridization assays. For example, RNA can be isolated and analyzed by Northern blot using a probe homologous to the CLASP-4 coding sequence or particular portions thereof. Alternatively, total nucleic acids of the host cell can be extracted and assayed for hybridization to such probes. Additionally, reverse transcription-polymerase chain reactions can be used to detect low levels of gene expression.

In the fourth approach, the expression of the CLASP-4 protein product can be assessed immunologically, for example by Western blots, immunoassays such as radioimmuno-precipitation, enzyme-linked immunoassays, fluorescent activated cell sorting (“FACS”), and the like. This can be achieved by using an anti-CLASP-4 antibody. Alternatively, CLASP-4 protein can be expressed as a fusion protein with green-fluorescent protein to facilitate its detection in cells (U.S. Pat. Nos. 5,491,084; 5,804,387; 5,777,079).

Identification of cells or tissues expressing CLASP protein or mRNA, especially CLASP-4 isoforms, can be useful for determining normal and abnormal CLASP expression in a given cell or tissue. As discussed above, a number of CLASP-4 isoforms have been identified, e.g., in Jurkat cells, peripheral blood, and brain. The identification of mRNA or protein expression in various cell types and tissues can allow for identification of isoforms improperly expressed in either a spatial or temporal manner.

### Uses of CLASP-4 Engineered Host Cells

In one embodiment of the invention, the CLASP-4 protein and/or cell lines that express CLASP-4 can be used to screen for antibodies, peptides, small molecules, natural and synthetic compounds or other cell bound or soluble molecules that bind to the CLASP-4 protein resulting in stimulation or inhibition of CLASP-4 function. For example, anti- CLASP-4 antibodies can be used to inhibit or stimulate CLASP-4 function and to detect its presence. Alternatively, screening of peptide libraries with recombinantly expressed soluble CLASP-4 protein or cell lines expressing CLASP-4 protein can be useful for identification of therapeutic molecules that function by inhibiting or stimulating the biological activity of CLASP-4. The uses of the CLASP-4 protein and engineered cell lines, described in the subsections below, can be employed equally well for homologous CLASP-4 genes in various species.

In a specific embodiment of the invention, cell lines may be engineered to express the extracellular or intracellular domain of CLASP fused to another molecule such as GST. In addition, CLASP, its extracellular domain or its intracellular domain may be fused to an immunoglobulin constant region (Hollenbaugh and Aruffo, 1992, Current Protocols in Immunology, Unit 10.19; Aruffo et al., 1990, Cell 61: 1303) to produce a soluble molecule with increased half life. The soluble protein or fusion protein can be used in binding assays, affinity chromatography, immunoprecipitation, Western blot, and the like. Synthetic compounds, natural products, and other sources of potentially biologically active materials can be screened in assays that are well known in the art.

Random peptide libraries consisting of all possible combinations of amino acids attached to a solid phase support can be used to identify peptides that are able to bind to a specific domain of CLASP-4 (Lam, K. S. et al., 1991, Nature 354: 82-84). The screening of peptide libraries can have therapeutic value in the discovery of pharmaceutical agents that stimulate or inhibit the biological activity of CLASP-4.

Identification of molecules that are able to bind to the CLASP-4 protein can be accomplished by screening a peptide library with recombinant soluble CLASP-4 protein. Methods for expression and purification of CLASP-4 are described in Section 5.7, supra, and can be used to express recombinant full length CLASP-4 or fragments of CLASP-4 depending on the functional domains of interest. Such domains include CLASP-4 extracellular domain, transmembrane domain, CLASP-4 intracellular domain, ITAM containing domain, tyrosine phosphorylation site containing domain, cysteine cluster containing domain, cadherin motif containing domain, and coil/coil domain.

To identify and isolate the peptide/solid phase support that interacts and forms a complex with CLASP-4, it is necessary to label or “tag” the CLASP-4 molecule. The CLASP-4 protein can be conjugated to enzymes such as alkaline phosphatase or horseradish peroxidase or to other reagents such as fluorescent labels which can include fluorescein isothiocyanate (FITC), phycoerythrin (PE) or rhodamine. Conjugation of any given label to CLASP-4 can be performed using techniques that are well known in the art. Alternatively, CLASP-4 expression vectors can be engineered to express a chimeric CLASP-4 protein containing an epitope for which a commercially available antibody exist. The epitope-specific antibody can be tagged with a detectable label using methods well known in the art including an enzyme, a fluorescent dye or colored or magnetic beads.

The “tagged” CLASP-4 conjugate is incubated with the random peptide library for 30 minutes to one hour at 22° C. to allow complex formation between CLASP-4 and peptide species within the library. The library is then washed to remove any unbound protein. If CLASP-4 has been conjugated to alkaline phosphatase or horseradish peroxidase the whole library is poured into a petri dish containing substrates for either alkaline phosphatase or peroxidase, for example, 5-bromo-4-chloro-3-indoyl phosphate (BCIP) or 3,3′,4,4″-diaminobenzidine (DAB), respectively. After incubating for several minutes, the peptide/solid phase- CLASP-4 complex changes color, and can be easily identified and isolated physically under a dissecting microscope with a micromanipulator. If a fluorescent tagged CLASP-4 molecule has been used, complexes can be isolated by fluorescence activated sorting. If a chimeric CLASP-4 protein expressing a heterologous epitope has been used, detection of the peptide/CLASP-4 complex can be accomplished by using a labeled epitope-specific antibody. Once isolated, the identity of the peptide attached to the solid phase support can be determined by peptide sequencing.

In addition to using soluble CLASP-4 molecules, in another embodiment, it is possible to detect peptides that bind to cell-associated CLASP-4 using intact cells. The use of intact cells is preferred for use with cell surface molecules. Methods for generating cell lines expressing CLASP-4 are described in Section 5.8. The cells used in this technique can be either live or fixed cells. The cells can be incubated with the random peptide library and bind to certain peptides in the library to form a “rosette” between the target cells and the relevant solid phase support/peptide. The rosette can thereafter be isolated by differential centrifugation or removed physically under a dissecting microscope. Techniques for screening combinatorial libraries are known in the art (Gallop et al., 1994, J. Med. Chem., 37: 1233; Gordon, 1994, J. Med. Chem., 37: 1385).

As an alternative to whole cell assays for membrane bound receptors or receptors that require the lipid domain of the cell membrane to be functional, CLASP-4 molecules can be reconstituted into liposomes where label or “tag” can be attached.

### CLASP-4 Fusion Proteins

In another embodiment of the invention, a CLASP-4 or a modified CLASP-4 sequence can be ligated to a heterologous sequence to encode a fusion protein. For example, for screening of peptide libraries for molecules that bind CLASP-4, it can be useful to produce a chimeric CLASP-4 protein expressing a heterologous epitope that is recognized by a commercially available antibody. A fusion protein can also be engineered to contain a cleavage site located between a CLASP-4 sequence and the heterologous protein sequence, so that the CLASP-4 can be cleaved away from the heterologous moiety. In one embodiment, fusion proteins of the invention can contain the CLASP-4 putative extracellular domain comprising at least about residues 1 through 1569 or fragments thereof. In another embodiment, fusion proteins can contain the CLASP-4 intracellular domain comprising at least about residue 843 of FIG. 1 (amino acid residue 1589 of FIG. 7) through the end of the CLASP-4 sequence or fragment thereof.

### Cloning Alleles, Variants, and Species Homologs of CLASP-4

In order to clone the full length cDNA sequence from any species encoding a CLASP-4 cDNA, or to clone variant forms of the molecule, labeled DNA probes made from nucleic acid fragments corresponding to any partial cDNA disclosed herein can be used to screen a cDNA library derived from lymphoid cells or brain cells. More specifically, oligonucleotides corresponding to either the 5′ or 3′ terminus of the cDNA sequence can be used to obtain longer nucleotide sequences. Briefly, the library can be plated out to yield a maximum of 30,000 pfu for each 150 mm plate. Approximately 40 plates can be screened. The plates are incubated at 37° C. until the plaques reach a diameter of 0.25 mm or are just beginning to make contact with one another (3-8 hours). Nylon filters are placed onto the soft top agarose and after 60 seconds, the filters are peeled off and floated on a DNA denaturing solution consisting of 0.4N sodium hydroxide. The filters are then immersed in neutralizing solution consisting of 1M Tris-HCl, pH 7.5, before being allowed to air dry. The filters are prehybridized in hybridization buffer such as casein buffer containing 10% dextran sulfate, 0.5M NaCl, 50 mM Tris-HCl, pH 7.5, 0.1% sodium pyrophosphate, 1% casein, 1% SDS, and denatured salmon sperm DNA at 0.5 mg/ml for 6 hours at 60° C. The radiolabeled probe is then denatured by heating to 95° C. for 2 minutes and then added to the prehybridization solution containing the filters. The filters are hybridized at 60° C for 16 hours. The filters are then washed in 1× wash mix (10× wash mix contains 3M NaCl, 0.6M Tris base, and 0.02M EDTA) twice for 5 minutes each at room temperature, then in 1× wash mix containing 1% SDS at 60° C. for 30 minutes, and finally in 0.3× wash mix containing 0.1% SDS at 60° C. for 30 minutes. The filters are then air dried and exposed to x-ray film for autoradiography. After developing, the film is aligned with the filters to select a positive plaque. If a single, isolated positive plaque cannot be obtained, the agar plug containing the plaques will be removed and placed in lambda dilution buffer containing 0.1M NaCl, 0.01M magnesium sulfate, 0.035M Tris HCl, pH 7.5, 0.01% gelatin. The phage can then be replated and rescreened to obtain single, well isolated positive plaques. Positive plaques can be isolated and the cDNA clones sequenced using primers based on the known cDNA sequence. This step can be repeated until a full length cDNA is obtained.

It can be necessary to screen multiple cDNA libraries from different tissues to obtain a full length cDNA. In the event that it is difficult to identify cDNA clones encoding the complete 5′ terminal coding region, an often encountered situation in cDNA cloning, the RACE (Rapid Amplification of cDNA Ends) technique can be used. RACE is a proven PCR-based strategy for amplifying the 5′ end of incomplete cDNAs. 5′-RACE-Ready RNA synthesized from human tissues containing a unique anchor sequence is commercially available (Clontech). To obtain the 5′ end of the cDNA, PCR is carried out on 5′-RACE-Ready cDNA using the provided anchor primer and the 3′ primer. A secondary PCR reaction is then carried out using the anchored primer and a nested 3′ primer according to the manufacturer's instructions. Once obtained, the full length cDNA sequence can be translated into amino acid sequence and examined for certain landmarks such as a continuous open reading frame flanked by translation initiation and termination sites, a cadherin-like domain, an ITAM domain, a tyrosine phosphorylation site, a cysteine cluster, a transmembrane domain, and finally overall structural similarity to the CLASP-4 genes disclosed herein. See, Ponassi et al., 1999, Mech. Dev. 80: 207-212; Isakov, 1998, Receptor Channels 5: 243-253; Borroto et al., 1997, Biopolymers 42: 75-88; Dimitratos et al., 1997, Mech. Dev. 63: 127-130; Apperson et al., 1996, J. Neurosci. 16: 6839-6852; Ozawa et al., 1990, Mech. Dev. 33: 49-56, which discuss protein domains and are incorporated herein by reference.

### Modulating Expression of Endogenous CLASP-4 Genes

Alternatively, the expression characteristics of an endogenous CLASP-4 gene within a cell population can be modified by inserting a heterologous DNA regulatory element into the genome of the cell line such that the inserted regulatory element is operatively linked with the endogenous CLASP-4 gene. For example, an endogenous CLASP-4 gene which is normally “transcriptionally silent”, i.e., an CLASP-4 gene which is normally not expressed, or is expressed only at very low levels in a cell population, can be activated by inserting a regulatory element which is capable of promoting the expression of a normally expressed gene product in the cells. Alternatively, a transcriptionally silent, endogenous CLASP-4 gene can be activated by insertion of a promiscuous regulatory element that works across cell types.

A heterologous regulatory element can be inserted into a cell line population, such that it is operatively linked with an endogenous CLASP-4 gene, using techniques, such as targeted homologous recombination, which are well known to those of skill in the art, (see e.g., in Chappel, U.S. Pat. No. 5,272,071; PCT publication No. WO 91/06667, published Jan. 16, 1991).

### Anti-CLASP-4 Antibodies

Various procedures known in the art can be used for the production of antibodies to epitopes of the natural and recombinantly produced CLASP-4 protein. Such antibodies include, but are not limited to, polyclonal, monoclonal, chimeric, single chain, human or humanized, IgG, IgM, IgA, IgD or IgE, a complementarity determining region, Fab fragments, F(ab')2 and fragments produced by an Fab expression library as well as anti-idiotypic antibodies. Antibodies which compete for CLASP-4 binding are especially preferred for diagnostics and therapeutics.

Monoclonal antibodies that bind CLASP-4 can be radioactively labeled allowing one to follow their location and distribution in the body after injection. Radioisotope tagged antibodies can be used as a non-invasive diagnostic tool for imaging de novo lymphoid tumors and metastases that express CLASP-4.

Immunotoxins can also be designed which target cytotoxic agents to specific sites in the body. For example, high affinity CLASP-4 specific monoclonal antibodies can be covalently complexed to bacterial or plant toxins, such as diphtheria toxin or ricin. A general method of preparation of antibody/hybrid molecules can involve use of thiol-crosslinking reagents such as SPDP, which attack the primary amino groups on the antibody and by disulfide exchange, attach the toxin to the antibody. The hybrid antibodies can be used to specifically eliminate CLASP-4 expressing lymphocytes.

For the production of antibodies, various host animals can be immunized by injection with the recombinant or naturally purified CLASP-4 protein, fusion protein or peptides, including but not limited to goats, rabbits, mice, rats, hamsters, and the like Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and poten-tially useful human adjuvants such as BCG (bacilli Calmette-Guerin) and Corynebacterium parvum.

Monoclonal antibodies to CLASP-4 can be prepared by using any technique which provides for the production of antibody molecules by continuous cell lines in culture. These include, but are not limited to, the hybridoma technique originally described by Kohler and Milstein, (Nature, 1975, 256: 495-497), the human B-cell hybridoma technique (Kosbor et al., 1983, Immunology Today, 4: 72; Cote et al., 1983, Proc. Natl. Acad. Sci. U.S.A., 80: 2026-2030) and the EBV-hybridoma tech-nique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A., 81: 6851-6855; Neuberger et al., 1984, Nature, 312: 604-608; Takeda et al., 1985, Nature, 314: 452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce CLASP-4 -specific single chain antibodies. In some embodiments, phage display technology is used to identify antibodies and heteromeric Fab fragments that specifically bind to selected antigens (see, e.g., McCafferty et al., Nature 348: 552-554 (1990); Marks et al., Biotechnology 10: 779-783 (1992)).

Hybridomas can be screened using enzyme-linked immunosorbent assays (ELISA) in order to detect cultures secreting antibodies specific for refolded recombinant CLASP-4. Cultures can also be screened by ELISA to identify those cultures secreting antibodies specific for mammalian-produced CLASP-4. Confirmation of antibody specificity can be obtained by western blot using the same antigens. Subsequent ELISA testing can use recombinant CLASP-4 fragments to identify the specific portion of the CLASP-4 molecule with which a monoclonal antibody binds. Additional testing can be used to identify monoclonal antibodies with desired functional characteristics such as staining of histological sections, immunoprecipitation of CLASP-4, inhibition of CLASP-4 binding or stimulation of CLASP-4 to transmit an intracellular signal. Determination of the monoclonal antibody isotype can be accomplished by ELISA, thus providing additional information concerning purification or function.

Some anti-CLASP-4 monoclonal antibodies of the present invention are humanized, human or chimeric, in order to reduce their potential antigenicity, without reducing their affinity for their target. Humanized antibodies have been described in the art. See, e.g., Queen, et al., 1989, Proc. Natl Acad. Sci. U.S.A. 86: 10029; U.S. Pat. Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., 1991, Protein Engineering 4: 773; Kolbinger et al., 1993, Protein Engineering 6: 971. Humanized monoclonal antibodies against CLASP-4 peptides can also be produced using transgenic animals having elements of a human immune system (see, e.g., U.S. Pat. Nos. 5,569,825; 5,545,806; 5,693,762; 5,693,761; and 5,7124,350).

In some embodiments, an anti-CLASP-4 polypeptide monoclonal or polyclonal antiserum is produced that is specifically immunoreactive with a particular CLASP-4 polypeptide and is selected to have low cross-reactivity against other molecules (e.g., other CLASP polypeptides) and any such cross-reactivity is removed by immunoabsorbtion prior to use in the immunoassay. Methods for screening and characterizing monoclonal antibodies for specificity are well known in the art and are described generally in Harlow and Lane, supra. For example, polyclonal antibodies raised to hCLASP-4, as shown in SEQ ID NO:2, or splice variants, or immunogenic portions thereof, can be selected to obtain only those polyclonal or monoclonal antibodies that are specifically immunoreactive with the target protein not with other proteins. This selection may be achieved by subtracting out antibodies that cross-react with molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background. Alternatively, antibodies that cross-react with a selected set of polypeptides may be prepared.

Antibody fragments which contain specific binding sites of V can be generated by known techniques. For example, such fragments include, but are not limited to, the F(ab')2 fragments which can be produced by pepsin digestion of the antibody molecule and the Fab fragments which can be generated by reducing the disulfide bridges of the F(ab')2 fragments. Alternatively, Fab expression libraries can be constructed (Huse et al., 1989, Science, 246: 1275-1281) to allow rapid and easy identification of monoclonal Fab fragments with the desired specificity to CLASP-4.

Anti-CLASP-4 antibodies can also be used to identify, isolate, inhibit or eliminate CLASP-4-expressing cells. In one embodiment, the present invention includes a method of identifying an abnormal T cell profile of an immunocompromised subject relative to the T cell profile of a non-immunocompromised subject. The method includes (i) sorting a sample of peripheral blood mononuclear cells (PBMC) isolated from the immunocompromised subject into sets of T cell types, (ii) determining the ratio of CLASP-4+ cells relative to the total number of cells (CLASP-4+: total) in each set, and identifying an abnormal T cell profile in the immunocompromised subject by comparing the CLASP-4+: total ratios of sets from the immunocompromised subject with the CLASP-4+: total ratios of analogous sets from a non-immunocompromised subject.

In other embodiments, anti-CLASP-4 antibodies can be used for detection of hCLASP-4 protein in assays such as fluorescent activated cell sorting (FACS), ELISA, fluorescent or electron immunomicroscopy, Western blots, gel shift analyses. CLASP-4 expression in various cells, localization within cells, interactions with other proteins, and differentiation between CLASP-4 isoform expression can be determined by use of the techniques listed herein.

### Screening Assays

The invention provides methods for identifying compounds or agents that modulate (i.e., inhibit or enhance) CLASP-4 expression or activity. CLASP-4 expression or activity modulators are useful for treatment of disorders characterized by (or associated with) aberrant or abnormal CLASP-4 expression or activity. Aberrant expression of CLASP-4 mRNA or protein means expression in lymphocytes (e.g., T lymphocytes or B lymphocytes) or other CLASP-4 expressing cells of at least 2-fold, preferably at least 5-fold greater than expression in control lymphocytes obtained from a healthy subject.

The CLASP-4 expression assays can include the steps of contacting a cell expressing CLASP-4 with a compound or agent and assaying CLASP-4 expression. CLASP-4 polypeptide expression is easily measured by ELISA using anti-CLASP-4 antibodies of the invention. CLASP-4 mRNA expression (including expression of specific species or splice variants of CLASP-4) can be measured by quantitative Northern analysis or quantitative PCR.

CLASP-4 activities include, for exampler, the CLASP-4 polypeptide CLASP-4 polypeptide involvement in signal transduction (e.g., leading to T cell activation). Compounds or agents that modulate the interaction of a CLASP-4 polypeptide and a target molecule, modulate CLASP-4 nucleic acid expression, or modulate CLASP-4 polypeptide activity are all contemplated by the methods of the present invention.

Test compounds include, for example, 1) peptides (e.g., soluble peptides, including Ig-tailed fusion peptides and members of random peptide libraries (see, e.g., Lam, K. S. et al., 1991, Nature 354: 82-84; Houghten, R. et al., 1991, Nature 354: 84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al., 1993, Cell 72: 767-778); 3) CLASP-4 antibodies (as described above); 4) small organic and inorganic molecules (e.g., molecules obtained from combinatorial and natural product libraries); 5) antisense RNA and DNA molecules and ribozymes (described above).

The CLASP modulators can be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds.

In one embodiment, the invention provides assays for screening test compounds which bind to CLASP-4 polypeptides. The assays can be recombinant cell based or cell-free assays. These assays can include the steps of combining a cell expressing a CLASP-4 polypeptide or a binding fragment thereof, and a compound or agent under conditions which allow binding of the compound or agent to the CLASP-4 polypeptide to form a complex. Complex formation can then be determined. The ability of the candidate compound or agent to bind to the CLASP-4 polypeptide or fragment thereof is indicated by the presence of the candidate compound in the complex. Formation of complexes between the CLASP-4 polypeptide and the candidate compound can be quantitated, for example, using standard immunoassays.

In another embodiment, the invention provides screening assays to identify test compounds which modulate the interaction (and most likely CLASP-4 activity as well) between a CLASP-4 polypeptide and a molecule (target molecule with which the CLASP-4 polypeptide normally interacts.

In one embodiment, these CLASP-4 target molecules can be tyrosine kinases (e.g., lyn, lck, fyn, ZAP-70m SyK, and CSK). In another embodiment, these CLASP-4 target molecules can be tyrosine phosphatases (e.g., EZRIN, SHP-1, SHP-2 and PTP36). In another embodiment, these CLASP-4 target molecules can be adaptor proteins (e.g., NCK, CBL, SHC, LNK, SLP-76, HS1, SIT, VAV, GrB2, and BRDG1). In another embodiment, these CLASP-4 target molecules can be cytoskeletal associated proteins such as ankyrin, spectrin, talin, ezrin, tropomyosin, myosin, plectin, syndecans, paralemmin, Band 3 protein, cytoskeletal protein 4.1, and PTP36. In a further embodiment, CLASP-4 target molecules can be members of the integrin family.

Typically, the assays are recombinant cell based or cell-free assay. These assays can include the steps of combining a cell expressing a CLASP-4 polypeptide or a binding fragment thereof, a CLASP-4 target molecule (e.g., a CLASP-4 ligand) and a test compound, under conditions where but for the presence of the candidate compound, the CLASP-4 polypeptide or biologically active portion thereof binds to the target molecule. Detecting complex formation between the CLASP-4 polypeptide or the binding fragment thereof, the CLASP-4 target molecule and a test compound detecting the formation of a complex which includes the CLASP-4 polypeptide and the target molecule can be accomplished. Detection of complex formation can include direct quantitation of the complex by, for example, measuring inductive effects, such as T cell activation, of the CLASP-4 polypeptide. A significant change, such as a decrease, in the interaction of the CLASP-4 and target molecule (e.g., in the formation of a complex between the CLASP-4 and the target molecule) in the presence of a candidate compound (relative to what is detected in the absence of the candidate compound) is indicative of a modulation of the interaction between the CLASP-4 polypeptide and the target molecule. Modulation of the formation of complexes between the CLASP-4 polypeptide and the target molecule can be quantitated using, for example, an immunoassay. To perform cell free drug screening assays, it is desirable to immobilize either CLASP-4 or its target molecule to facilitate separation of complexes from uncomplexed forms of one or both of the polypeptides, as well as to accommodate automation of the assay. CLASP-4 binding to a target molecule, in the presence and absence of a candidate compound, can be accomplished in any vessel suitable for containing the reactants. Examples of such vessels include microtitre plates, test tubes, and microcentrifuge tubes.

In one embodiment, a fusion polypeptide can be provided which adds a domain that allows the polypeptide to be bound to a matrix. Alternatively, the complexes can be dissociated from the matrix, separated by SDS-PAGE, and the level of CLASP-4-binding polypeptide found in the bead fraction quantitated from the gel using standard electrophoretic techniques.

Other techniques for immobilizing polypeptides on matrices can also be used in the drug screening assays of the invention. For example, either CLASP-4 or its target molecule can be immobilized utilizing conjugation of biotin and streptavidin. Biotinylated CLASP-4 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with CLASP-4 but which do not interfere with binding of the polypeptide to its target molecule can be derivatized to the wells of the plate, and CLASP-4 trapped in the wells by antibody conjugation. As described above, preparations of a CLASP-4 -binding polypeptide and a candidate compound are incubated in the CLASP-4 -presenting wells of the plate, and the amount of complex trapped in the well can be quantitated. Methods for detecting such complexes include immunodetection of complexes using antibodies reactive with the CLASP-4 target molecule, or which are reactive with CLASP-4 polypeptide and compete with the target molecule; as well as enzyme-linked assays which rely on detecting an enzymatic activity associated with the target molecule.

One method of drug screening utilizes eukaryotic or prokaryotic host cells which are stably transformed with recombinant DNA molecules expressing the CLASP-4, e.g., the protein having the sequence of SEQ ID NO:2. Such cells, either in viable or fixed form, can be used for standard ligand/receptor binding assays (see, e.g., Parce et al. (1989) Science 246: 243-247; and Owicki et al. (1990) Proc. Natl Acad. Sci. U.S.A. 87: 4007-4011, which describe sensitive methods to detect cellular responses. A test compound, often labeled, can be assayed for binding or for competition with another ligand for binding. Viable cells could also be used to screen for the effects of drugs on CLASP-4 mediated functions, e.g., T cell activation, second messenger levels, and others).

In another embodiment, the invention provides a method for identifying a compound (e.g., a screening assay) capable of use in the treatment of a disorder characterized by (or associated with) aberrant or abnormal CLASP-4 nucleic acid expression or CLASP-4 polypeptide activity. This method typically includes the step of assaying the ability of the compound or agent to modulate the expression of the CLASP-4 nucleic acid or the activity of the CLASP-4 polypeptide thereby identifying a compound for treating a disorder characterized by aberrant or abnormal CLASP-4 nucleic acid expression or CLASP-4 polypeptide activity.

Methods for assaying the ability of the compound or agent to modulate the expression of the CLASP-4 nucleic acid or activity of the CLASP-4 polypeptide are typically cell-based assays. For example, cells which are sensitive to ligands which transduce signals via a pathway involving CLASP-4 can be induced to overexpress a CLASP-4 polypeptide in the presence and absence of a candidate compound. Candidate compounds which produce a change in CLASP-4-dependent responses can be identified. In one embodiment, expression of the CLASP-4 nucleic acid or activity of a CLASP-4 polypeptide is modulated in cells and the effects of candidate compounds on the readout of interest (such as T cell activation) are measured. For example, the expression of genes which are up- or down-regulated in response to a CLASP-4-dependent signal cascade can be assayed.

Alternatively, modulators of CLASP-4 expression can be identified in a method where a cell is contacted with a candidate compound and the expression of CLASP-4 mRNA or polypeptide in the cell is determined. The level of expression of CLASP-4 mRNA or polypeptide in the presence of the candidate compound is compared to the level of expression of CLASP-4 mRNA or polypeptide in the absence of the candidate compound. The candidate compound can then be identified as a modulator of CLASP-4 nucleic acid expression based on this comparison. For example, when expression of CLASP-4 mRNA or polypeptide is greater in the presence of the candidate compound than in its absence, the candidate compound is identified as a stimulator of CLASP-4 nucleic acid expression. Alternatively, when CLASP-4 nucleic acid expression is less in the presence of the candidate compound than in its absence, the candidate compound is identified as an inhibitor of CLASP-4 nucleic acid expression. The level of CLASP-4 nucleic acid expression in the cells can be determined by methods described herein for detecting CLASP-4 mRNA or polypeptide.

Modulators of CLASP-4 polypeptide activity and CLASP-4 nucleic acid expression identified according to these drug screening assays can be used to treat, for example, immune disorders. These methods of treatment include the steps of administering the modulators of CLASP-4 polypeptide activity or nucleic acid expression, e.g., in a pharmaceutical composition as described in §5.10.1 below, to a subject in need of such treatment, e.g., a subject with a disorder described herein.

### Therapeutic Administration of CLASP-4 Modulators

The CLASP-4 protein is expressed in lymphocytes and, as noted supra, play a role in regulating T cell and B cell interactions, thus making CLASP-4 activity (e.g., CLASP-4 binding of regulatory proteins) a target for diagnostic and treatment of immune disorders and for modulation of immune function (e.g., T cell activation). Additionally, since CLASP-4 contains domains capable of transducing an intracellular signal, cell surface CLASP-4 can be triggered by an anti- CLASP-4 antibody or soluble CLASP-4 or a fragment thereof in order to enhance the activation state of a lymphocyte.

### Formulation and Route of Administration

A CLASP-4 polypeptide, a fragment thereof, anti-CLASP-4 antibody, CLASP-4 polynucleotide (e.g., antisense or ribozyme), or small molecule agonists or antagonists can be administered to a subject per se or in the form of a pharmaceutical or therapeutic composition. Pharmaceutical compositions comprising the proteins of the invention can be manufactured by means of conventional mixing, dissolving, granulating, dragee-making, levigating, emulsifying, encapsulating, entrapping or lyophilizing processes. Pharmaceutical compositions can be formulated in conventional manner using one or more physiologically acceptable carriers, diluents, excipients or auxiliaries which facilitate processing of the protein or active peptides into preparations which can be used pharmaceutically. Proper formulation is dependent upon the route of administration chosen.

Currently, there are three major classes of protein-derived cell-penetrating peptides that have been used for delivering of proteins into cells and animals (Lindgren, M.; et al., 2000, Trends Pharmacol Sci. 21: 99-103). In one embodiment, the CLASP-4 protein or fragment (encoding a functional domain of CLASP-4) can be introduced into the cell as a fusion protein tied to a transporter protein derived from homeoprotein transcription factors such as ANTP. In another embodiment, the CLASP-4 protein or fragment (encoding a functional domain of CLASP-4) can be introduced into the cell as a fusion protein tied to other transcription factors such as the HIV Tat protein and the herpes simplex virus type 1 (HSV-1) VP22 protein. Members in this family have been widely used in different cellular and animal systems (Schwarze, S.; et al.; 2000, Trends Pharmacol Sci. 21: 45-48). In another embodiment, the CLASP-4 protein or fragment (encoding a functional domain of CLASP-4) can be introduced into the cell as a fusion protein tied to peptides derived from signal-sequences present in several proteins such as HIV-1 gp41. In other embodiments, there are several synthetic and/or chemeric cell-penetrating peptides such as transportan and Amphiphiloc model peptide (Lindgren, M.; et al., 2000, Trends Pharmacol Sci. 21: 99-103) that can be used. In another embodiment, the CLASP-4 protein or fragment can be introduced by using anti-DNA antibodies (see, e.g., Zack, D. J., et al., 1996, J. Immunol. 157: 2082-8

For topical administration the proteins of the invention can be formulated as solutions, gels, ointments, creams, suspensions, and the like. as are well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration.

For injection, the proteins of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, the proteins can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, a composition can be readily formulated by combining the proteins with pharmaceutically acceptable carriers well known in the art. Such carriers enable the proteins to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents can be added, such as the cross-linked polyvinylpyrrolidone, agar, or alginic acid or a salt thereof such as sodium alginate.

If desired, solid dosage forms can be sugar-coated or enteric-coated using standard techniques.

For oral liquid preparations such as, for example, suspensions, elixirs and solutions, suitable carriers, excipients or diluents include water, glycols, oils, alcohols, and the like. Additionally, flavoring agents, preservatives, coloring agents and the like can be added.

For buccal administration, the proteins can take the form of tablets, lozenges, and the like. formulated in conventional manner.

For administration by inhalation, the proteins for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The proteins can also be formulated in rectal or vaginal compositions such as suppositories or retention enemas, e.g., containing conventional suppository bases such as cocoa butter or other glycerides.

In addition to the formulations described previously, the proteins can also be formulated as a depot preparation. Such long acting formulations can be administered by implantation (for example subcutaneously or intramuscularly) or by intramuscular injection. Thus, for example, the proteins can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver the proteins or peptides of the invention. Certain organic solvents such as dimethylsulfoxide also can be employed, although usually at the cost of greater toxicity. Additionally, the proteins can be delivered using a sustained-release system, such as semipermeable matrices of solid polymers containing the therapeutic agent. Various sustained-release materials have been established and are well known by those skilled in the art. Sustained-release capsules can, depending on their chemical nature, release the proteins for a few weeks up to over 100 days. Depending on the chemical nature and the biological stability of the therapeutic reagent, additional strategies for protein stabilization can be employed.

As the proteins and peptides of the invention can contain charged side chains or termini, they can be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

### Effective Dosages

CLASP-4 polypeptides, CLASP-4 fragments and anti-CLASP-4 antibodies will generally be used in an amount effective to achieve the intended purpose. For use to inhibit an immune response, the proteins of the invention, or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC50 as determined in cell culture (i.e., the concentration of test compound that inhibits 50% of CLASP-4 binding interactions). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval can be adjusted individually to provide plasma levels of the proteins which are sufficient to maintain therapeutic effect. Usual patient dosages for administration by injection range from about 0.1 to 5 mg/kg/day, preferably from about 0.5 to 1 mg/kg/day. Therapeutically effective serum levels can be achieved by administering multiple doses each day.

In cases of local administration or selective uptake, the effective local concentration of the proteins can not be related to plasma concentration. One having skill in the art will be able to optimize therapeutically effective local dosages without undue experimentation.

The amount of CLASP-4 administered will, of course, be dependent on the subject being treated, on the subject's weight, the severity of the affliction, the manner of administration and the judgment of the prescribing physician.

The therapy can be repeated intermittently while symptoms detectable or even when they are not detectable. The therapy can be provided alone or in combination with other drugs. In the case of autoimmune disorders, the drugs that can be used in combination with CLASP-4 or fragments thereof include, but are not limited to, steroid and non-steroid immunosuppressive agents.

### Toxicity

Preferably, a therapeutically effective dose of the proteins described herein will provide therapeutic benefit without causing substantial toxicity.

Toxicity of the proteins described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch.1, p.1).

### Binding Assays

CLASP-4 polypeptides can be used to screen for molecules that bind to CLASP-4 or for molecules to which CLASP-4 binds. The binding of CLASP-4 by the molecule can activate (agonist), increase, inhibit (antagonist), or decrease activity of the CLASP-4 or the molecule bound. Examples of such molecules include antibodies, oligonucleotides, proteins (e.g., receptors), or small molecules. Preferably, the molecule is closely related to the natural ligand of CLASP-4, e.g., a fragment of the ligand, or a natural substrate, a ligand, a structural or functional mimetic. (See, Coligan et al., Current Protocols in Immunology 1(2): Chapter 5 (1991).) Similarly, the molecule can be closely-related to the natural receptor to which CLASP-4 binds, or at least, a fragment of the receptor capable of being bound by CLASP-4 (e.g., active site). In either case, the molecule can be rationally designed using known techniques.

Preferably, the screening for these molecules involves producing appropriate cells which express CLASP-4, either as a secreted protein or on the cell membrane. Preferred cells include cells from mammals, yeast, Drosophila, or E. coli. Cells expressing CLASP-4 (or cell membrane containing the expressed polypeptide) are then preferably contacted with a test compound potentially containing the molecule to observe binding, stimulation, or inhibition of activity of either CLASP-4 or the molecule.

The assay can simply test binding of a candidate compound to CLASP-4, where binding is detected by a label, or in an assay involving competition with a labeled competitor. Further, the assay can test whether the candidate compound results in a signal generated by binding to CLASP-4.

Alternatively, the assay can be carried out using cell-free preparations, polypeptide affixed to a solid support, chemical libraries, or natural product mixtures. The assay can also simply comprise the steps of mixing a candidate compound with a solution containing CLASP-4, measuring CLASP-4 activity or binding, and comparing the CLASP-4 activity or binding to a standard. Preferably, an ELISA assay can measure CLASP-4 level or activity in a sample (e.g., biological sample) using a monoclonal or polyclonal antibody. The antibody can measure CLASP-4 level or activity by either binding, directly or indirectly, to CLASP-4 or by competing with CLASP-4 for a substrate.

In another aspect of the invention, the CLASP-4 polypeptides, or fragments thereof, can be used as “bait proteins” in a two-hybrid assay (see, e.g., U.S. Pat. No. 5,283,317; Zervos et al., 1993, Cell 72: 223-232; Madura et al., 1993, J. Biol. Chem. 268: 12046-12054; Bartel et al., 1993, Biotechniques 14: 920-924; Iwabuchi et al., 1993, Oncogene 8: 1693-1696; and Brent WO 94/10300), to identify other proteins, which bind to or interact with CLASP-4 (“CLASP-4-binding proteins” or “CLASP-4-bp”) and modulate CLASP-4 polypeptide activity. Such CLASP-4-binding proteins are also likely to be involved in the propagation of signals by the CLASP-4 polypeptides as, for example, upstream or downstream elements of the CLASP-4 pathway.

All of these above assays can be used as diagnostic or prognostic markers. The molecules discovered using these assays can be used to treat disease or to bring about a particular result in a patient by activating or inhibiting the CLASP-4 molecule. Moreover, the assays can discover agents which can inhibit or enhance the production of CLASP-4 from suitably manipulated cells or tissues.

Therefore, the invention includes a method of identifying compounds or agents that bind to CLASP-4 polypeptides comprising the steps of: (a) contacting a CLASP-4 polypeptide with a compound or agent under conditions which allow binding of the compound to the CLASP-4 polypeptide to form a complex and (b) determining if binding has occurred. Moreover, the invention includes a method of identifying agonists or antagonists comprising the steps of: (a) incubating a candidate compound with CLASP-4, (b) assaying a biological activity, and (b) determining if a biological activity of CLASP-4 has been altered.

Several methods of automating assays have been developed in recent years so as to permit screening of tens of thoU.S.A.nds of compounds in a short period. See, e.g., Fodor et al., 1991, Science 251: 767-773, and other descriptions of chemical diversity libraries, which describe means for testing of binding affinity by a plurality of compounds.

### Other Uses of CLASP-4 Polynucleotides and Polypeptides

The polynucleotides, polypeptides, polypeptide homologues, modulators, and antibodies described herein can be used in one or more of the following methods: a) drug screening assays; b) diagnostic assays particularly in disease identification, allelic screening and pharmocogenetic testing; and c) pharmacogenomics. A CLASP-4 polypeptide of the invention has one or more of the activities described herein and can thus be used to, for example, modulate an immune response in an immune cell, for example by binding to a CLASP-4 binding partner making it unavailable for binding to the naturally present CLASP-4 polypeptide.

In one embodiment, these CLASP-4 binding partners can be tyrosine kinases (e.g., lyn, Ick, fyn, ZAP-70m SyK, and CSK). In another embodiment, these CLASP-4 binding partners can be tyrosine phosphatases (e.g., EZRIN, SHP-1, SHP-2 and PTP36). In another embodiment, these CLASP-4 target molecules can be adaptor proteins (e.g., NCK, CBL, SHC, LNK, SLP-76, HS1, SIT, VAV, GrB2, and BRDG1. In another embodiment, these CLASP-4 binding partners can be cytoskeletal associated proteins such as ankyrin, spectrin, talin, ezrin, tropomyosin, myosin, plectin, syndecans, paralemmin, Band 3 protein, cytoskeletal protein 4.1, and PTP36. In a further embodiment, CLASP-4 binding partners can be members of the integrin family.

The isolated nucleic acid molecules of the invention can be used to express CLASP-4 polypeptide (e.g., via a recombinant expression vector in a host cell or in gene therapy applications), to detect CLASP-4 mRNA (e.g., in a biological sample) or a naturally occurring or recombinantly generated genetic mutation in an CLASP-4 gene, and to modulate CLASP-4 activity, as described further below. In addition, the CLASP-4 polypeptides can be used to screen drugs or compounds which modulate CLASP-4 polypeptide activity as well as to treat disorders characterized by insufficient production of CLASP-4 polypeptide or production of CLASP-4 polypeptide forms which have decreased activity compared to wild type CLASP-4. Moreover, the anti-CLASP-4 antibodies of the invention can be used to detect and isolate an CLASP-4 polypeptide, particularly fragments of CLASP-4 present in a biological sample, and to modulate CLASP-4 polypeptide activity.

### Diagnostic Assays

The invention further provides a method for detecting the presence of CLASP-4, or fragment thereof, in a biological sample. Usually the biological sample contains lymphocytes (e.g., from blood). The method involves contacting the biological sample with a compound or an agent capable of detecting CLASP-4 polypeptide or mRNA such that the presence of CLASP-4 is detected in the biological sample.

A preferred agent for detecting CLASP-4 mRNA is a directly or indirectly labeled nucleic acid probe capable of hybridizing to CLASP-4 mRNA. The nucleic acid probe can be, for example, the full-length CLASP-4 cDNA of SEQ ID NO:1, or a portion thereof, such as an oligonucleotide of at least 15, 30, 50, 100, 250 or 500 nucleotides in length and sufficient to specifically hybridize under stringent conditions to CLASP-4 mRNA.

A preferred agent for detecting CLASP-4 polypeptide is a directly or indirectly labeled antibody capable of binding to a CLASP-4 polypeptide. Antibodies can be polyclonal, or more preferably, monoclonal. An intact antibody, or a fragment thereof (e.g., Fab or F(ab)2) can be used. The term “directly or indirectly”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The detection method of the invention can be used to detect CLASP-4 mRNA or polypeptide in a biological sample in vitro as well as in vivo. For example, in vitro techniques for detection of CLASP-4 mRNA include Northern hybridizations and in situ hybridizations. In vitro techniques for detection of CLASP-4 polypeptide include enzyme linked immunosorbent assays (ELISAs), Western blots, immunoprecipitations and immunofluorescence. Alternatively, CLASP-4 polypeptide can be detected in vivo in a subject by introducing into the subject a labeled anti-CLASP-4 antibody. For example, the antibody can be labeled with a radioactive marker whose presence and location in a subject can be detected by standard imaging techniques. Particularly useful are methods which detect the allelic variant of CLASP-4 expressed in a subject and methods which detect fragments of an CLASP-4 polypeptide in a sample.

The invention also encompasses kits for detecting the presence of CLASP-4 in a biological sample. For example, the kit can comprise a directly or indirectly labeled compound or agent capable of detecting CLASP-4 polypeptide or mRNA in a biological sample; means for determining the amount of CLASP-4 in the sample; and means for comparing the amount of CLASP-4 in the sample with a standard. The compound or agent can be packaged in a suitable container. The kit can further comprise instructions for using the kit to detect CLASP-4 mRNA or polypeptide.

The methods of the invention can also be used to detect naturally occurring genetic mutations in an CLASP-4 gene, thereby determining if a subject with the mutated gene is at risk for a disorder characterized by aberrant or abnormal CLASP-4 nucleic acid expression or CLASP-4 polypeptide activity as described herein. In preferred embodiments, the methods include detecting, in a sample of cells from the subject, the presence or absence of a genetic mutation characterized by at least one of an alteration affecting the integrity of a gene encoding an CLASP-4 polypeptide, or the misexpression of the CLASP-4 gene.

### Biological Activities of CLASP-4

As described herein, CLASP-4 mediates a variety of cell functions in lymphocytes and other cells. As described herein, a variety of assays are useful for detecting or quantitating CLASP-4 activity, or for identifying agents (including polynucleotides, polypeptides, and antibodies of the invention) that modulate CLASP-4 activity (i.e., biological activity, e.g., binding) or expression. Such agents are useful for treatment of diseases and conditions associated with aberrant CLASP-4 expression or activity. Further, following the guidance provided herein, other CLASP-4-mediated activities can be identified by those of skill using routine assays, such as those described below.

Exemplary assays for CLASP-4 function (or modulation of function) include assays for modulation of an in vitro or in vivo cell response (e.g., an immune response such as lymphocyte activation, antibody production, inflammation) by detecting a change in an activity (e.g., cytokine production, calcium flux, tyrosine phosphorylation, regulation of early activation markers, cell metabolism, proliferation, and the like, as described below) of cells in vitro or in vivo. In one embodiment, the cells are lymphocytes.

In one assay, for example, recombinant CLASP-4 protein, peptides, or antibodies corresponding to the CLASP-4 extracellular domain can be mixed directly with T and B cells. Cytokine production by these cells can then be measured and the degree of modulation of the immune response quantitated. Alternatively, antigen-presenting B cells are mixed with untransfected T cells or T cells that have been transfected with CLASP-4 isoforms. Cytokine production (or calcium flux or other assays described below) is be measured at the appropriate time to determine the effect of CLASP-4 on such an immune response. In a similar assay, B cells transfected with CLASP-4 constructs are tested for their ability to stimulate a T cell to generate an immune response. Transfected constructs in any of these cases could encode, for example, full or partial length CLASP-4 sequences, or antisense constructs to inhibit translation of endogenous CLASP-4 gene. Any of the examples described herein can be used to stimulate an immune response in the presence or absence of CLASP-4 isoforms or antibodies and assay the resulting effects on immune response by the methods listed below.

### Methods for Generating an Immune Response in vitro

In various assays, an effect of an agent on immune cells is detected using an in vitro assay. The degree of an immune response can be measured or quantitated by a number of standard assays including those described below.

In one assay, human peripheral blood mononuclear cells (PBMC), human T cell clones (e.g., Jurkat E6, ATCC TIB-152), EBV-transformed B cell clones (e.g., 9D10, ATCC CRL-8752), antigen-specific T cell clones or lines can be used to examine immune responses in vitro. Activation, enhanced activation or inhibition of activation of these cells or cell lines can be used for the evaluation of potential CLASP therapeutics. Standard methods by which hematopoietic cells are stimulated to undergo activation characteristic of an immune response are, for example:

A) Antigen specific stimulation of immune responses. Either pre-immunized or naïve mouse splenocytes can be generated by standard procedures. In addition, antigen-specific T cell clones and hybridomas (e.g., MBP-specific), and numerous B cell lymphoma cell lines (e.g., CH27), have been previously characterized are available for the assays discussed below. Antigen specific splenocytes or B-cells can be mixed with specific T-cells in the presence of antigen to generate an immune response. This can be performed in the presence or absence of CLASP-4 to assay whether CLASP-4 modulates the immune response as measured by any of the assays in below.

B) Non-specific T cell activation. The following methods can be used to activate T cells in the absence of antigen: 1) cross-linking T cell receptor (TCR) by addition of antibodies against receptor activation molecules (e.g., TCR, CD3, or CD2) together with antibodies against co-stimulator molecules, for example anti-CD28; 2) activating cell surface receptors in a non-specific fashion using lectins such as concanavalin A (con A) and phytohemagglutinin (PHA); 3) mimicking cell surface receptor-mediated activation using pharmacological agents that activate protein kinase C (e.g., phorbol esters) and increase cytoplasmic Ca2+(e.g., ionomycin).

C) Non-specific B cell activation: 1) application of antibodies against cell surface molecules such as IgM, CD20, or CD21. 2) Lipopolysaccharide (LPS), phorbol esters, calcium ionophores and ionomycin can also be used to by-pass receptor triggering.

D) Mixed lymphocyte reaction (MLR). Mix donor PBMC with recipient PBMC to activate lymphocytes by presentation of mismatched tissue antigens, which occurs in all cases except identical twins.

E) Generation of a specific T cell clone or line that recognizes a particular antigen. A standard approach is to generate tetanus toxin-specific T cells from a donor that has recently been boosted with tetanus toxin. Major histocompatability complex- (MHC-) matched antigen presenting cells and a source of tetanus toxin are used to maintain antigen specificity of the cell line or T cell clone (Lanzavecchia, A., et al., 1983, Eur. J. Immun. 13: 733-738).

The anticipated mechanism of action of a CLASP-4 polypeptide or polynucleotide should define the appropriate assay to use to investigate its potential enhancement or inhibition of lymphocyte activation. For example, soluble proteins containing the CLASP extracellular domain may interfere with the interaction between T cells and antigen presenting cells. Such interaction plays a role in the MLR and in antigen-specific T cell activation, but not in non-specific T or B cell activation. The assays described above have the advantage of several possible detection methods for quantitation.

### Methods for Generating an Immune Response in vivo

In various assays, an effect of an agent on immune cells is detected using an in vivo assay. The degree of an immune response can be measured or quantitated by a number of standard assays including those described below.

(A) Animal Model for Transplantation Rejection: Ectopic Heart Transplantation

In one embodiment, a standard animal model for graft versus host rejection is ectopic heart transplantation (Fulmer et al., 1963, Am. J. Anat. 113: 273-281). This method involves using BALB/C mice (either sex, and range from 1-9 months) for transplanting cardiac tissue into a surgically-created pocket on the dorsum for both ears made by slitting the skin over the auricular artery at the base of the ear. Small curved forceps are forced into the slit, bluntly dissecting between the skin and the cartilage plate. Donor tissue is eased into the base of the pocket near the distal edge of the ear. The auricular artery is used to seal off the opening of the pocket. Within 10 to 14 days pulsatile activity of the transplant should be observed. Gross appearance of the graft, patterns of vacuolar supply to the graft area and pulsatile activity can be easily observed utilizing transilluminated light during the first three weeks post-transplantation. Follow-up can continue for for several months.

(B) Animal model for Autoimmune Disease: Induction of Collagen Induced Arthritis (CIA)

Collagen Induced Arthritis (CIA) is a standard model for studying progression and immune (Courtenay et al., 1980, Nature 283: 666 and Wooley et al., 1981, J. Exp. Med. 154: 688). DBA/a mice can be used as an assay for the in vivo relevance of CLASP-4 in vitro testing potential immune therapeutics. In vivo experiments will be performed to examine the ability of potential therapeutics to prevent CIA. We will use 3-5 mice per group to statistically justify our results.

Once a titer of the potency of collagen type II (CII) is obtained therapeutics can be tested. In one embodiment, three mice will be immunized with three different concentrations of CII 50, 200, and 400 μg per animal (Nabozny et al.., 1996, J. Exp. Med., 183: 27-37). To induce CIA, animals can be immunized with an appropriate concentration of CII, determined as described above. One half of a 1:1 ratio of antigen:CFA can be injected at the base of the tail and the remainder equally divided in each hind footpad. Mice can be carefully monitored every day for the onset and progression of CIA thoughout the experiment until its termination 12 weeks post-immunization with CII. The pieces of heart transplanted can be approximately 3×3 mm in size. The severity of arthritis can be assessed following standard procedures known to one of skill in the art.

### Assay Quantitation

(A) Tyrosine phosphorylation

Tyrosine phosphorylation of early response proteins such as HS1, PLC-r, ZAP-76, and Vav is an early biochemical event following T cell activation. The tyrosine phosphorylated proteins can be detected by Western blot using antibodies against phosphorylated tyrosine residues. Tyrosine phosphorylation of these early response proteins can be used as a standard assay for T cell activation (J. Biol. Chem., 1997, 272(23): 14562-14570). Any change in the phosphorylation pattern of these or related proteins when immune responses are generated in the presence of CLASP-4 is indicative of a CLASP-4 modulation of this response.

(B) Intracellular Calcium Flux

The kinetics of intracellular Ca2+ concentrations are measured over time after stimulation of cells preloaded with a calcium sensitive dye. Upon binding the Ca2+ indicator dye, Fluor-4 (Molecular Probes), exhibits an increase in fluorescence level using flow cytometry, solution fluorometry, and confocal microscopy. Any change in the level or timing of calcium flux when immune responses are generated in the presence of CLASP-4 is indicative of a CLASP-4 modulation of this response

(C) Regulation of early activation markers

Increased and diminished expression/regulation of early lymphocyte activation marker levels such as CD69, IL-2R, MHC class II, B7, and TCR are commonly measured with fluorescently labeled antibodies using flow cytometry. All antibodies are commercially available. Any change in the expression levels of lymphocyte activation markers when immune responses are generated in the presence of CLASP-4 is indicative of a CLASP-4 modulation of this response.

(D) Increased metabolic activity/acid release

Activation of most known signal transduction pathways trigger increases in acidic metabolites. This reproducible biological event is measured as the rate of acid release using a microphysiometer (Molecular Devices), can be used as an early activation marker when comparing the treatment of cells with potential biological therapeutics (McConnell, H. M. et al., 1992, Science 257: 1906-1912 and McConnell, H. M., 1995, Proc. Natl. Acad. Sci. 92: 2750-2754). Any statistically significant increase or decrease in acid release of CLASP-4-treated sample, as compared to control sample (no treatment), suggest and effect of CLASP-4 on biological function.

(E) Cell proliferation/cell viability assays

(1) ³H-thimidine incorporation

Exposure of lymphocytes to antigen or mitogen in vitro induces DNA synthesis and cellular proliferation. The measurement of mitotic activity by 3H-thimidine incorporation into newly synthesized DNA is one of the most frequently used assays to quantitative T cell activation. Depending on the cell population and form of stimulation used to activate the T cells, mitotic activity can be measured within 24-72 hrs. in vitro, post 3H-thimidine pulse (Mishell, B. B. and S. M. Shiigi, 1980, Selected Methods in Cellular Immunology, W. H. Freeman and Company and Dutton, R. W. and Pearce, J. D., 1962, Nature 194: 93). Any statistically significant increase or decrease in CPM of CLASP-4-treated sample, as compared to control sample (no treatment), suggest and effect of CLASP-4 on biological function.

(2) MTS [5-(3-carboxymethoxyphenyl)-2-(4,5-dimethylthiazolyl)-3(4-sulfophenyl)tetrazolium, inner salt] is a calorimetric method for determining the number of viable cells in proliferation or cytotoxicity assays (Barltrop, J. A. et al., 1991, Bioorg. & Med. Chem. Lett. 1: 611). 1-5 days after lymphocyte activation, MTS tetrazolium compound, Owen's reagent, is bioreduced by cells into a colored formazan product that is soluble in tissue culture media. Color intensity is read at 490 nm minus 650 nm using a microplate reader. Any statistically significant increase or decrease in color intensity of CLASP-4-treated sample, as compared to control sample (no treatment), can suggest an effect of CLASP-4 on biological function (Mosmann, T., 1983, J. Immunol. Methods 65: 55 and Barltrop, J. A. et al. (1991)).

(3) Bromodeoxyuridine (BrdU), a thymidine analogue, readily incorporates into cells undergoing DNA synthesis. BrdU-pulsed cells are labeled with an enzyme-conjugated anti-BrdU antibody (Gratzner, H. G., 1982, Science 218: 474-475.). A colorimetric, soluble substrate is used to visualize proliferating cells that have incorporated BrdU. Reaction is stopped with sulfuric acid and plate is read at 450 nm using a microplate reader. Any statistically significant increase or decrease in color intensity of CLASP-4-treated sample, as compared to control sample (no treatment), suggest an effect of CLASP-4 on biological function.

(F) Apoptosis by Annexin V

Programmed cell death or apoptosis is an early event in a cascade of catabolic reactions leading to cell death. A lose in the integrity of the cell membrane allows for the binding of fluorescently conjugated phosphatidylserine. Stained cells can be measured by fluorescence microscopy and flow cytometry (Vennes, I., 1995, J. Immunol. Methods. 180: 39-52). In one embodiment, any statistically significant increase or decrease in apoptotic cell number of CLASP-4-treated sample, as compared to control sample (no treatment), suggest an effect of CLASP-4 on biological function. For evaluating apoptosis in situ, assays for evaluating cell death in tissue samples can also be used in vivo studies.

(G) Quantitation of cytokine production

Cell supernatants harvested after cell stimulation for 16-48 hrs are stored at −80° C. until assayed or directly tested for cytokine production. Multiple cytokine assays can be performed on each sample. IL-2, IL-3, IFN-γ and other cytokine ELISA Assays are available for mouse, rat, and human (Endogen, Inc. and BioSource). Cytokine production is measured using a standard two-antibody sandwich ELISA protocol as described by the manufacturer. The presence of horseradish peroxidase is detected with 3, 3′5, 5′ tertamethyl benziidine (TMB) substrate and the reaction is stopped with sulfuric acid. The absorbency at 450 mm is measured using a microplate reader. Any statistically significant increase or decrease in color intensity of CLASP-4-treated sample, as compared to control sample (no treatment), suggest an effect of CLASP-4 on biological function.

(H) NF-AT can be visualized by Immunostaining

T cell activation requires the import of nuclear factor of activated T cells (NFAT) to the nucleus. This translocation of NF-AT can be visualized by immunostaining with anti-NF-AT antibody (Cell 1998, 93: 851-861). Therefore, NF-AT nuclear translocation has been used to assay T cell activation. Similarly, NF-AT/luciferase reporter assays have been used as a standard measurement of T cell activation (MCB 1996, 12: 7151-7160).

(I) ELISA for collagen type II (CII)-specific antibodies (see above for related in vivo assay)

C(II) titers from serum of animals immunized with CLASP-4 can be measured and compared. Both TH1-dependent IgG2a and TH2-dependent IgGI and IgE CII-specific antibody isotypes will be measured by ELISA. Mouse blood will be obtained by orbital bleed one and two months post-immunization with CII. Samples will be allowed to coagulate and centrifuge to obtain sera, and stored at −80° C. until assayed by ELISA. Coat ELISA plates with CII and dilute sera. HRP conjugated goat, isotype specific antibody. Plates are then expose to TMB substrate and read at 450 nm using a microplate reader (Nabozny et al., 1996, J. Exp. Med. 183: 27-37). Any change in the levels of Collagen specific antibodies by calorimetric test when immune responses are generated in the presence of CLASP-4 is indicative of a CLASP-4 modulation of this response.

(J) Antibody Production by ELISPOT Assay

A solid-phase enzyme-linked immunospot (ELISPOT) assay for the quantification of isotype-specific antibody secreting cells (Czerkinsky et al., 1983, J Immunol. Methods. 65: 109-121). Both human and mouse B cells can be tested for isotype and antigen specific antibody production. Although based on a standard ELISA, this technique becomes more sensitive by detecting antibody secretion from single cells. Any change in ELISPOT levels when immune responses are generated in the presence of CLASP-4 is indicative of a CLASP-4 modulation of this response.

(K) Cellular degranulation following IgE cross-linking.

Two cell lines have been obtained from ATCC (MEG10 and HEL-17.92), both of which express the human FCεR1 receptor. FCεR1 is the high affinity receptor for IgE complexes, which when coupled to biotin can be cross-linked with avidin to induce degranulation and histamine release of lymphocytes. Following acylatation of the sample, histamine is quantified with an enzyme immunoassay competition assay (Immunotech). Histamine release. A statistically significant increase or decrease in histamine concentration of a CLASP-4 treated sample, as compared to control sample (no treatment), suggest an effect of CLASP-4 on biological function. Any change in frequency of degranulation or histamine levels when immune responses are generated in the presence of CLASP-4 is indicative of a CLASP-4 modulation of this response.

(L) Cellular phenotyping of lymphocytes by flow cytometry and Immunocytochemistry

Determining the tissue distribution of lymphocytes following a pathological disorder can aid in identifying specific organ, tissue and lymphocyte involved in an immune response. Cellular phenotyping of lymphocyte trafficking is generally performed with by flow cytometry and Immunocytochemistry. There are several cluster determination (CD) molecules that are routinely used to identify phenotype, activation kinetics, and regulation events of cells. Any change in levels or distribution of CD molecules when immune responses are generated in the presence of CLASP-4 is indicative of a CLASP-4 modulation of this response.

(M) Structure/Function Assays: Homotypic and/or Heterotypic, Calcium-dependant Cell Adhesion

L929 cells can be transfected with CLASP-4 and Neomycin. G418-resistant clones can be screened for CLASP-expression with anti-CLASP peptide-specific antibodies. These CLASP-expressing clones can then be used to test for homotypic and/or heterotypic calcium dependent cell adhesion using the “cell aggregation assay” described for cadherin molecules (Murphy-Erdosh, C. et al., 1995, J. Cell Biol. 129: 1379-1390). Any change in the levels of cellular aggregation when immune responses are generated in the presence of CLASP-4 is indicative of a CLASP-4 modulation of this response.

The following cDNA clones described in the Specification and further described in the Examples below have been deposited with the American Type Culture Collection, 10801 University Boulevard, Manassas, Va. 20110-2209 under the Budapest Treaty on Mar. 24, 2000 and given the Accession Nos. indicated:

hCLASP-4 3′ clone (AVC-PD4) ATCC Accession Number PTA-1566

hCLASP-4 5′ clone (AVC-PD11) ATCC Accession Number PTA-1572.

The following cDNA clones described in the Specification and further described in the Examples below have been deposited with the American Type Culture Collection, 10801 University Boulevard, Manassas, Va. 20110-2209 under the Budapest Treaty on Oct. 17, 2000 and given the Accession Nos. indicated:

hCLASP-4 clone C4P2AS4 (AVC-PD16) ATCC Accession Number PTA-2608

hCLASP-4 clone hC4S5-AS12 (AVC-PD17) ATCC Accession Number PTA-2610.

### EXAMPLES

### EXAMPLE 1

Cloning of CLASP-4

The cloning of the CLASP gene family has not been a straghtforward process. The cloning of each CLASP family member required the use of multiple techniques and resources. CLASP-4 was cloned in the following manner: an expressed sequence tag or EST clone (IMAGE clone 968931, derived from 12 week human embryo) was identified based on a BLAST search of human GenBank human EST database using CLASP-1 sequences. IMAGE clone 968931 was sequenced completely. A polynucleotide probe prepared from 968931 sequence was labeled with ³²P-dCTP and used to screen human cDNA libraries including Jurkat (Stratagene) and Ramos B cell cDNA library (James Boulter, UCLA). The screening methods employed were as described in Maniatis et al., 1989, Molecular Cloning A Laboratory Manual, Cold Spring Harbor Laboratory, New York. Two clones were identified, cloned, sequenced (ABI dye-sequencing system, PE Applied Biosystems; Perkin-Elmer Corporation, 761 Main Avenue, Norwalk, Conn., U.S.A.) and were aligned to generate a contiguous cDNA sequence with 3477 base pairs, which was incomplete. Commercial libraries from multiple tissue sources including human placenta, B cell, T cell and peripheral blood were exhaustively screened and re-screened resulting in the acquisition of only partial cDNAs. Generation of cDNA libraries using oligo dT or CLASP-specific primers also resulted in the acquisition of partial cDNAs. Genomic libraries were screened to obtain a portion of the genomic locus for each of the CLASP genes, and a genomic walk was initiated to obtain 5′ exons and extend the cDNA sequence.

A genomic BAC library was screened (Genome Systems, St. Louis, Mo.) and a BAC containing sequence homologous to the 5′ portion of our sequence identified at that point was found (BAC human release 1 filters, filter 1, section 1, well 2d, plate 1). This turned out to encode an intronless pseudogene copy of hCLASP-4 with multiple mutations in the coding region that resulted in predicted nonsense, missense and frameshift mutations. Primers were designed from this sequence and used to amplify expressed hCLASP-4 cDNAs using RT-PCR. These were distinct from the genomic pseudogene sequence and represented a hCLASP-4 gene that must be expressed from an independent locus.

To obtain additional 5′ CLASP-4 sequence, portions of the cDNA and genomic sequence from a BAC (Bacterial Artificial Chromosome) genomic library were compared to the NCBI database by BLAST. A genomic clone (Genbank identifier: gi7248268) was identified that contained the sequence of the intronless pseudogene that represented a mutated copy of human CLASP-4. Using TFASTX (Pearson and Lipman, PNAS (1988) 85:2444-2448), the amino-terminal sequence of human CLASP-4 was compared to 6 frame translation of GI:3876325 (C. elegans homolog). Areas of gi8158664 that encoded amino acids with high similarity to the C. elegans GI:3876325 amino acid sequence were used to design CLASP-4-specific oligonucleotides for RTPCR (reverse transcriptase polymerase chain reaction according to manufacturers instructions: Reverse transcriptase Gibco/BRL, Taq Polymerase from Sigma). Using sense oligonucleotides such as C4gS5 (5′ CAGAGATCTGCTTATGTTTC; nucleotides −13 to +6 of FIG. 5, 2 mismatches) and antisense oligonucleotide C4AS12 (5′ CCAACAAGTATGTGGCCATC; reverse complement of nucleotides 2718 to 2737 of FIG. 5 - one mismatch) an RTPCR product of approximately 2.7 kb was generated, sequenced (dideoxytnucleotide termination sequencing, Beckman Coulter CEQ2000) and shown to be additional human CLASP-4 5′ sequence from a locus other than the pseudogene. Further complicating the cloning full-length CLASP cDNA products was the difficulty to clone (and subclone) certain CLASP cDNA products. Standard isolation of some of the CLASP cDNAs from a pure phage population following screening of commercially available cDNA libraries (“ZAP-out” procedure, Stratagene) resulted in no bacterial colonies. Similarly, certain RT-PCR products could not be cloned into standard plasmid vectors. No colonies were isolated by cloning these fragments into vectors lacking promoters, reverse orientations, low copy vectors, or by growth at altered temperatures or levels of antibiotic for plasmid selection (examples: CLASP-7 - HC7gS6 to HC7gAS1 and HC7gS3 to HC7AS14; CLASP-4 - C4P2 to hC4ASTM and C4P2 to HC4AS3′; CLASP-1 - hClS5′ to hC1AS3′Kpn and C1S7 to hC1AS3′Kpn; see Primer Table below). One possibility is that sequences contained within certain regions of CLASP cDNAs are bacteriacidal and therefore not amenable to cloning. To circumvent these problems direct sequencing of RT-PCR products was performed.

PRIMER TABLE

| | | | | |
|---|---|---|---|---|
| CLASP | Sense | | Antisense | |
| gene | Primer | Sense sequence | Primer | Antisense sequence |

| | | | | |
|---|---|---|---|---|
| CLASP-7 | HC7gS5 | AGGCCTTGTCTCTGTTTACCTG | HC7gAS1 | TGTCATGTACTGCACTCGCACAGC |
| CLASP-7 | HC7gS3 | ACAGGAACCTGCTGTACGTGTAC | HC7AS14 | TCGTGGCTGCACAGGATGCGGGTG |
| CLASP-4 | C4P2 | GACCCATTAGGAGGTCTAC | HC4AS3' | CGGGATCCATTGTCACCGTACATCT |
| | | | | GC |
| CLASP-4 | C4P2 | GACCCATTAGGAGGTCTAC | HC4AS3' | CGGGATCCATTGTCACCGTACATCT |
| | | | | GC |
| CLASP-1 | hC1S5' | TATGTCTCAGTCACCTACCTG | HC1AS3'Kpn | CTTGGTACCACTTCAGCACTAGATG |
| | | | | AGATG |
| CLASP-1 | C1S7 | TCAAGACCAGGGCATGCAAG | HC1AS3'Kpn | CTTGGTACCACTTCAGCACTAGATG |
| | | | | AGATG |

In-frame stop codons were not present suggesting that the cDNA was not full length. To obtain the 5′ terminus of CLASP-4, 5′ RACE was employed. Antisense oligonucleotides directed against the 5′ end of the longest CLASP-4 sequence were generated:

Primers used for human CLASP-4 5′ RACE

| Primer sequence(5′ TO 3′) | nucleotide position |
|---|---|
| hC4RACE2 | |
| GACGGCGCATTTTGGGGCACTGAACAAC | 490-517 |
| HC4RACE1 | |
| CTGCACCGTTCTGCGCTGACGACCTATC | 33-60 |

RACE was carried out using Invitrogen's Generacer kit according to manufacturers specifications with polyA selected mRNA from 9D 10 and MV411 B cell tissue culture lines. The sequence of the oligonucleotides presented is the reverse complement (i.e. antisense) of the the CLASP-4 cDNA at the indicated position based upon numbering in FIG. 5. Many RACE clones were isolated and completely sequenced resulting in the identification of 10 potential start sites for transcription. These were used to search genbank for additional genomic information resulting in the identification of gi5836219, a genomic clone of 122674 bp that contains the promoter region and exons 2 through 17 (FIG. 6A). Upon mapping these potential transcriptional start sites it was noted that the fall into 5 distinct overlapping groups, several of which are delineated in FIG. 5A and FIG. 6B.

The full length cDNA sequence (presented in FIG. 5) is therefore a compilation of overlapping sequences isolated from cDNA libraries, RT-PCR products and 5′ RACE products.

### EXAMPLE 2

Expression of human CLASP-4 in Mammalian cells

Expression of HC4 was evaluated in different cell lines using DNA transfection of tagged fusion protein constructs. We used two standard methods for DNA transfection, DNA precipitation by calcium phosphate (Graham, F. and van der Eb, A. and Gorman, C. et al.,) and electroporation (Potter, H.). The expression system was used to analyze CLASP expression in mouse and human lymphocytic cells and in human 293 cells was based on red fluorescent protein (RFP).

Material and Methods

Cell lines used for transfection of CLASP-tagged constructs

Jurkat E6 human T cells, (ATCC TIB-152), were maintained and tested in complete IMDM (IMDM medium supplemented with 2 mM glutamine, 100 U/mL penicillin, 100 μg/mL streptomycin, 0.1 mM nonessential amino acids, 1 mM sodium pyruvate (Gibco BRL), 50 μM beta mercaptoethanol (Sigma), and 10% fetal calf serum (Gemini Bio-Products)). Human embryonic kidney cells, 293,were maintained and tested in complete DMEM (DMEM medium supplemented with 2 mM glutamine, 10 mM HEPES, 100 U/mL penicillin, 100 μg/mL streptomycin, and 10% fetal calf serum). CH27 mouse B cell lymphoma and 2B4 mouse T cell hybrid were maintained and tested in complete RPMI (RPMI 1640 medium supplemented with 2 mM glutamine, 100 U/mL penicillin, 100 μg/mL streptomycin, 0.1 mM nonessential amino acids, 1 mM sodium pyruvate, 10 mM HEPES, 50 μM beta mercaptoethanol, and 10% fetal calf serum).

Transfection Methods

DNA precipitation by calcium phosphate was used to transfect 293 cells. At least 2 hrs before transfection, ˜5×10⁵ cells, in 5 ml of complete DMEM, were plated onto one 60 mm TC-treated dish for each transfection. For each plate, 1-10 μg of DNA was brought to a volume of 110 μl with deionized H2O. Fifteen μl of 2M CaCl₂ was added to the DNA solution, which was then slowly added to 125 μl of 2× HBS pH 7.0, (2× HBS=1.64% NaCl 1.188% and Hepes 0.04% Na₂HPO₄). A fine precipitate formed, which was then added dropwise to the plate of cells and incubated at 37 C, 5% CO₂. Cells were analyzed for expression on day 1, 2 and 3 post-transfection (see Analysis of CLASP-tagged constructs).

The lymphocytic cell lines (Jurkat E6, CH27, and 2B4) cells were transfected by electroporation and tested for protein expression on day 1 and 2 post-transfection. The BTX ECM830 generator was used to transfect the Jurkat E6 cells as follows: two cuvettes, with a 4 mm electrode gap, containing 5×10⁶ cells in 0.5 ml serum free-IMDM and 5-30 μg of DNA, were electroporated with a single pulse at 260 volts for 50 msec. Cuvettes were immediately placed on ice for 10-15 minutes before being transferred to 10 ml of complete medium. CH27, and 2B4 cells were transfected in cuvettes with a 4 mm electrode gap, using the BioRad Gene pulser. The protocol using the BioRad Gene pulser is the same as for the BTX ECM830 generator except that they were electroporated in serum free-RPMI @ 0.45kV, 960 μF, and unlimited resistance. The time constant using the BioRad electroporator ranged from 38-44.

Analysis of CLASP-tagged fusion constructs

RFP-tagged plasmid

A red fluorescent protein (RFP) vector, DsRED (Clonetech), was used to generate HC4-RFP-fusion proteins. The putative HC4 regions tested for expression in the DsRED vector cover the following regions: tm=transmembrane, D=dock domains, CC=coil-coil region, and 3′ c-terminal region. The specific constructs made and tested for RFP expression contained the following CLASP regions: 42-1 HC4 (tm-D), 43-2 HC4 (tm-CC), 44-1 HC4 (tm-3′), 46-1 HC4 (D-CC), 47-1 HC4 (D-3′), and 49-1 HC4 (CC-3′). 50-1 HC4 (CC-3) plasmid did not contain RFP and was therefore used as a negative control. The lymphocytic cell lines (Jurkat E6, CH27, and 2B4 cells) were transfected by electroporation and tested for RFP-tagged-plasmid expression on day 1 and 2 post-transfection. HC4-RFP-fusion proteins were tested using a Coulter EPICS XL flow cytometer and an inverted Nikon Diaphot fluorescent microscope.

RFP-tagged expression by flow cytometry and fluorescent microscopy

Jurkat E6 cells were found to have maximal expression of RFP control plasmid on day 1 post-transfection, whereas 293 cells had maximal expression on day 2. All HC4-RFP-tagged construct results below are for these time points. The DsRED plasmid expressed RFP in both Jurkat E6 and 293 cells but none of the HC4-RFP-tagged constructs were expressed in Jurkat E6 cells when tested either by flow cytometry or fluorescent microscopy. 293 cells did express 42-1 HC4 (tm-D), 43-2 HC4 (tm-CC), 44-1 HC4 (tm-3′), 46-1 HC4 (D-CC), and 49-1 HC4 (CC-3). The percent expression range was 15-55%. The HC4-RFP-tagged fusion protein 47-1 HC4 (D-3′) was not expressed in 293 cells. As expected, in the negative control, 50-1 HC4 (CC-3), expression of RFP was not detected by either method.

Co-transfection of Jurkat E6 with GFP and HC4-RFP fusion constructs (previously shown to not to express in Jurkat E6 cells) gave GFP expression levels equal to transfection levels from GFP only, thus suggesting that the HC4-RFP constructs are not toxic to Jurkat E6 cells.

Fluorescent microscopy revealed different expression patterns for each construct expressed in 293 cells on day 2 post-transfection. The RFP expression from the control DsRED construct was evenly diffused in the cell varying from weak to very bright fluorescence. The 42-1 HC4 (tm-D) staining pattern was polarized and globular, whereas 43-2 HC4 (tm-CC) and 44-1 HC4 (tm-3′) had occasional rings and were both more tightly polarized (than 42-1). 46-1 HC4 (D-CC) was differentially speckled and 49-1 HC4 (CC-3) was diffuse and punctated. There was no fluorescent RFP detected in 293 cells transfected with 47-1 HC4 (D-3′).

Discussion

Given the lack of expression of truncated forms of human CLASP-4 using an RFP-tagged expression system, it can be concluded that the level of CLASP expression is highly regulated in mouse and human lymphocytes, but not in human 293 cells. This regulation is at the Level of translation or post-translation, possibly affecting a lymphocyte process necessary for cell survival, protein expression, or sequestering proteins away from where they are needed for these processes.

References

Graham, F. and van der Eb, A., (1973). Virology 52:456.

Gorman, C., Science, (1983). 221, 551-553.

Potter, H. (1995). Recombinant DNA Methodology II. Academic Press, Inc. Chapter 31, pg. 467-484. Applications of Electroporation in Recombinant DNA Technology.

### EXAMPLE 31

### Expression of human CLASP-4 in activated T-cells

General experimental design

The expression profiles of human CLASP-4 in T cells upon T cell activation was determined by Northern analysis. Jurkat E6 lymphoblasts were activated by treatment with anti-CD28, PMA, and Ionomycin. Subsequently, total RNA was extracted from cell aliquots harvested at 0, 1, 2, 4, 8, and 14 hours post activation. The RNA concentration of each preparation was determined by the absorption at 260 nm using a spectrophotometer and concentrations of the different RNA preparations were adjusted such that equal quantities of each RNA preparation could be subjected to Northern analysis. Even gel loading was monitored by ethidium bromide staining of the formaldehyde-agarose gel. Northern membranes were hybridized to radioactively labeled probes corresponding to portions of human CLASP-4 and human beta-actin. Expression levels of CLASP-4 at different time points post T-cell activation are proportional to the radioactive signal generated by hybridization by the CLASP-4 specific radioactively labeled probe that remained bound to the Northern membrane under stringent washing conditions. The entire experiment was done in duplicate.

Jurkat E6 cell activation

Jurkat E6 cells were maintained and tested in complete IMDM medium supplemented with 2 mM glutamine, 10 mM HEPES, 100 u/mL penicillin, 100 μg/mL streptomycin, 0.1 mM nonessential amino acids, 1 mM sodium pyruvate (Gibco/BRL), 50 μM beta mercaptoethanol (Sigma), and 10% fetal calf serum (Gemini). T cells were activated as described per Fraser et. al., using 0.1 μg/mL mouse anti-human CD28 monoclonal antibody (PharMingen International catalog number 33741A), 50 ng/mL PMA (Sigma), and 1 μM ionomycin (Calbiochem). Following incubation at 37° C. and 5.0% v/v CO₂, 0.5×10⁶ cells were harvested by centrifugation at 500× g for 10 minutes (min) at room temperature at 0, 1, 2, 4, 8 and 14 hours post activation and subjected to RNA extraction.

For RNA preparation, probe labelling and Northern analysis protocols, see methods and procedures described in Example 2 above. Primers 129333R3(S) and 129333F2(AS) were used to generate the probe spanning from N 4958 to N 5634 of CLASP-4.

Hybridization, Washing, and Exposure

Blots were washed twice in 2× SSC 0.1% SDS for 10 min each at 60° C. and then twice in 0.2× SSC 0.1% SDS for 10 min each at 60° C., followed by a 5′ wash in 2×SSC at 60° C. Exposure to KODAK BIOMAX MS film was carried out at minus 80° C. using amplifying screens. Typically, exposure times were 10 to 36 hours. Signal intensities on Northern membranes were quantified by the use of a phosphor imager system (STORM, Molecular Dynamics). Signals were counted in the “volume report” mode.

Blots were washed twice in 2× SSC 0.1% SDS for 10 min each at 60° C. and then twice in 0.2× SSC 0.1% SDS for 10 min each at 60° C., followed by a 5′ wash in 2×SSC at 60° C. Exposure to KODAK BIOMAX MS film was carried out at minus 80° C. using amplifying screens. Typically, exposure times were 10 to 36 hours.

Signal intensities on Northern membranes were quantified by the use of a phosphor imager system (STORM, Molecular Dynamics). Signals were counted in the “volume report” mode.

Results

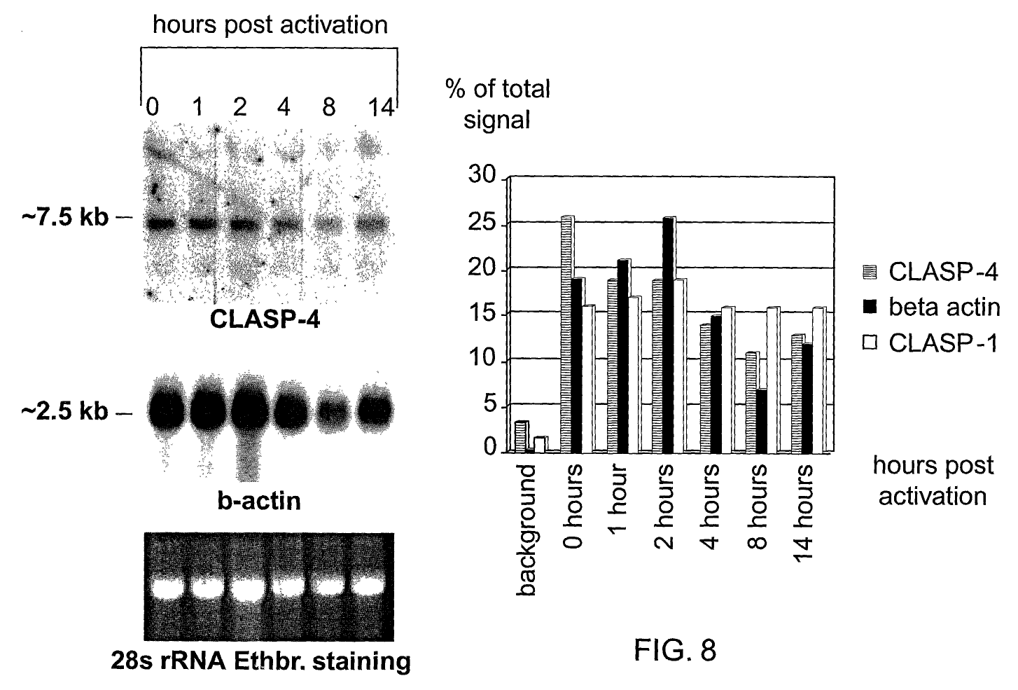

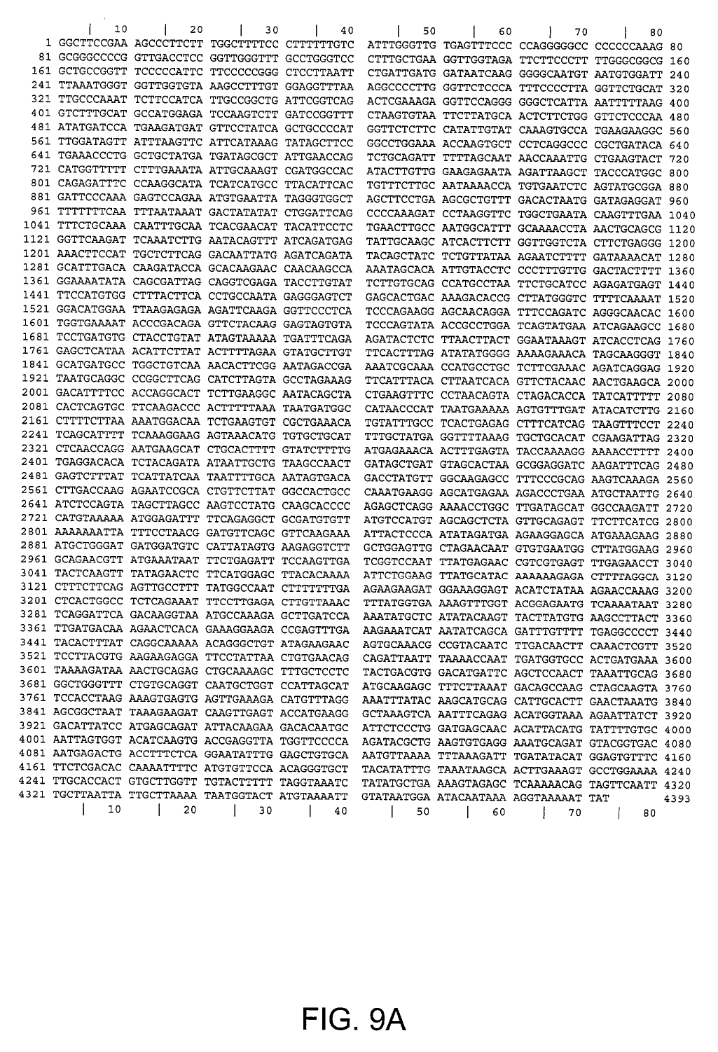

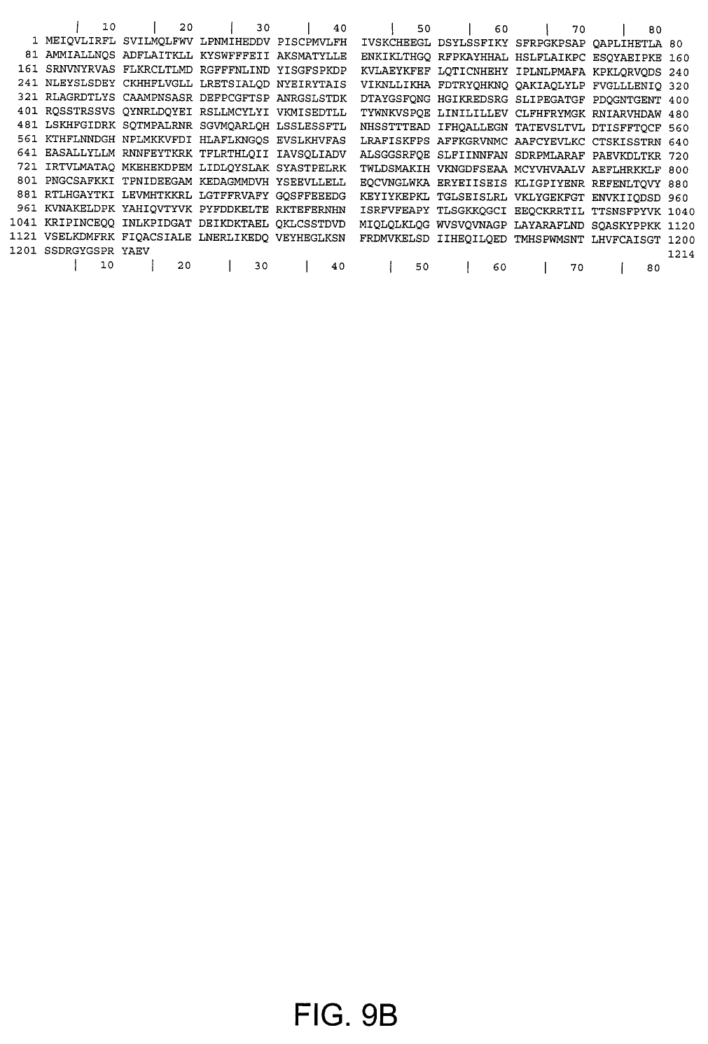

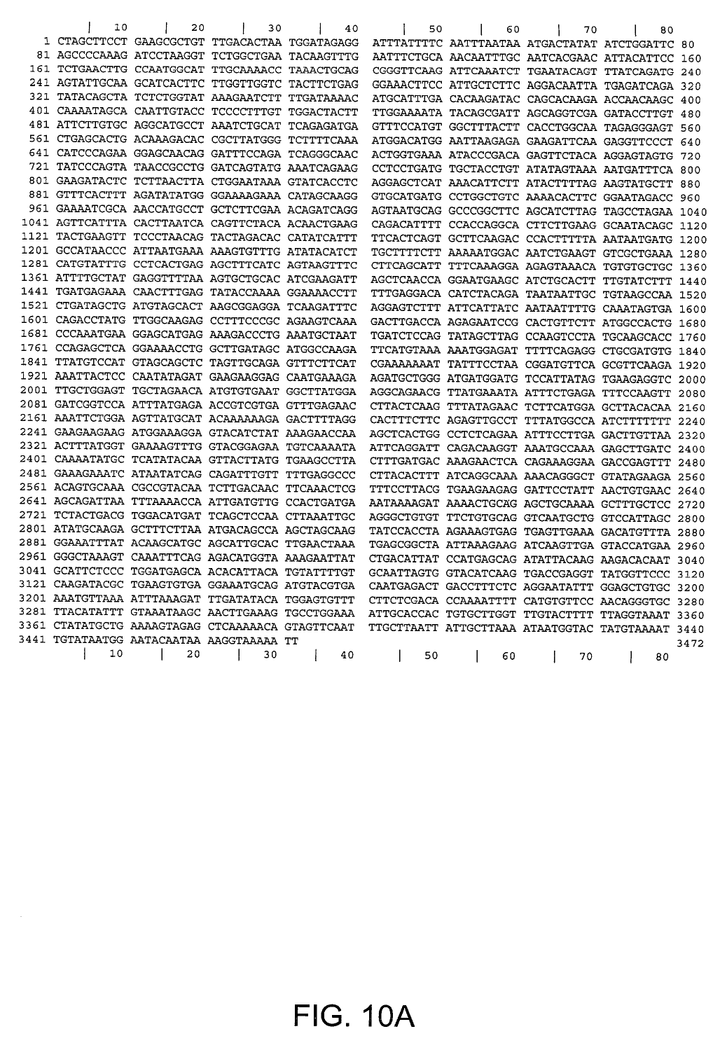

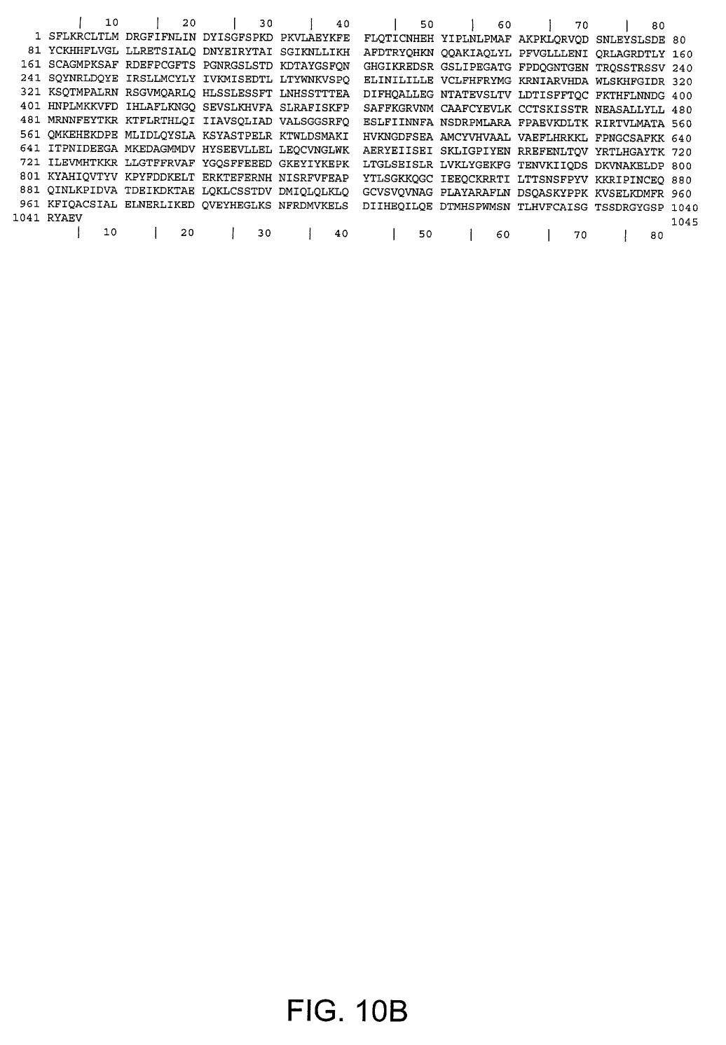

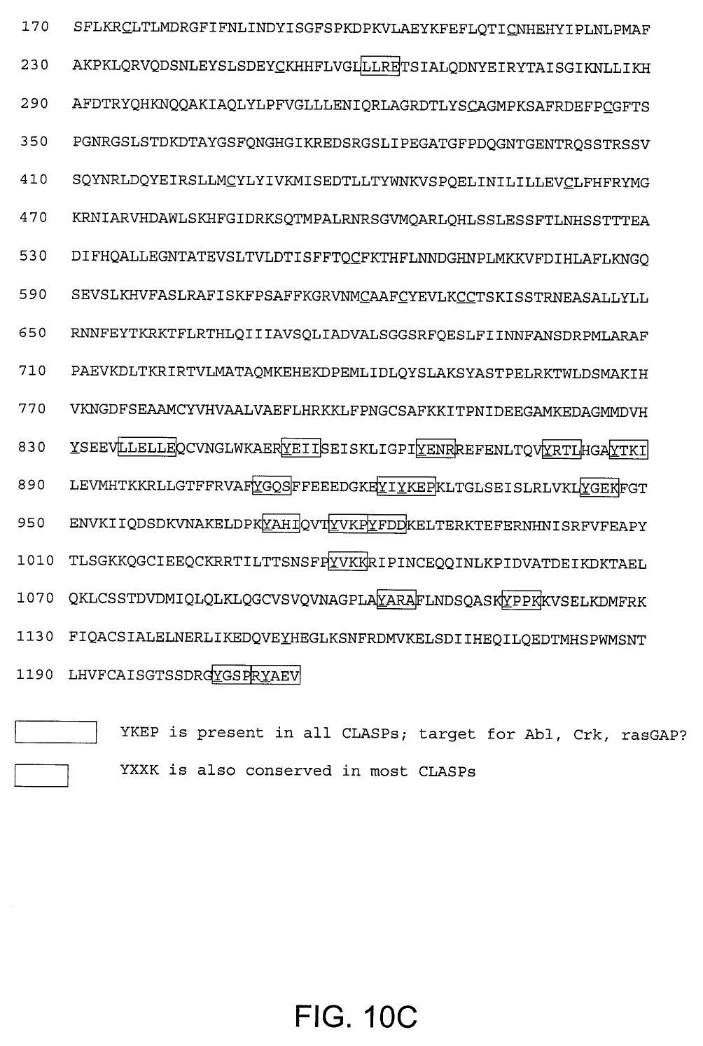

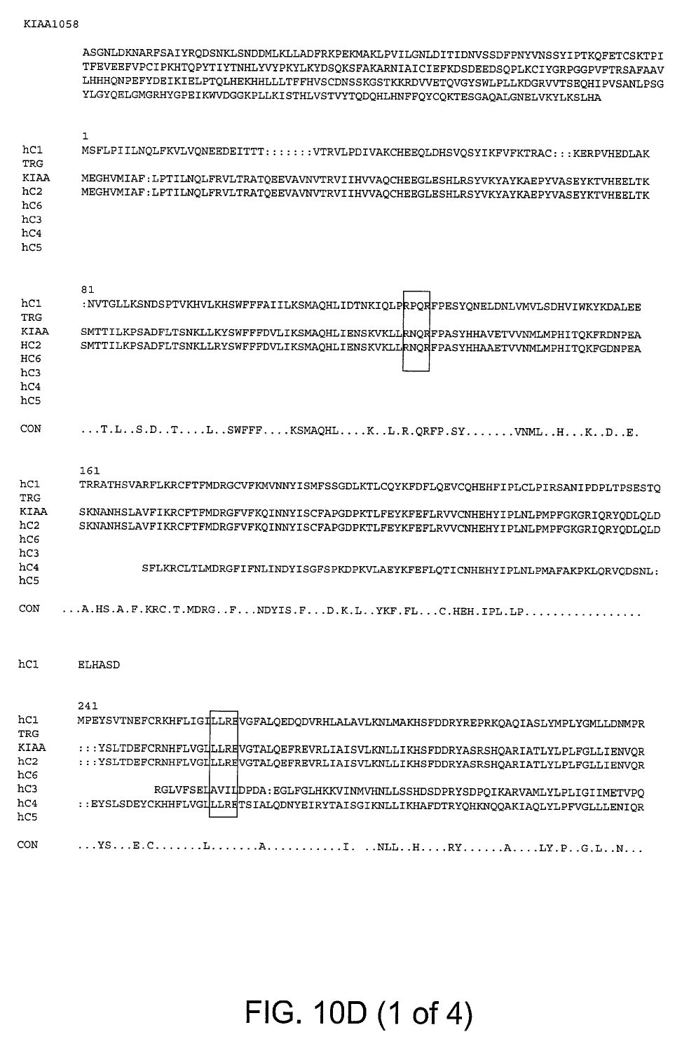

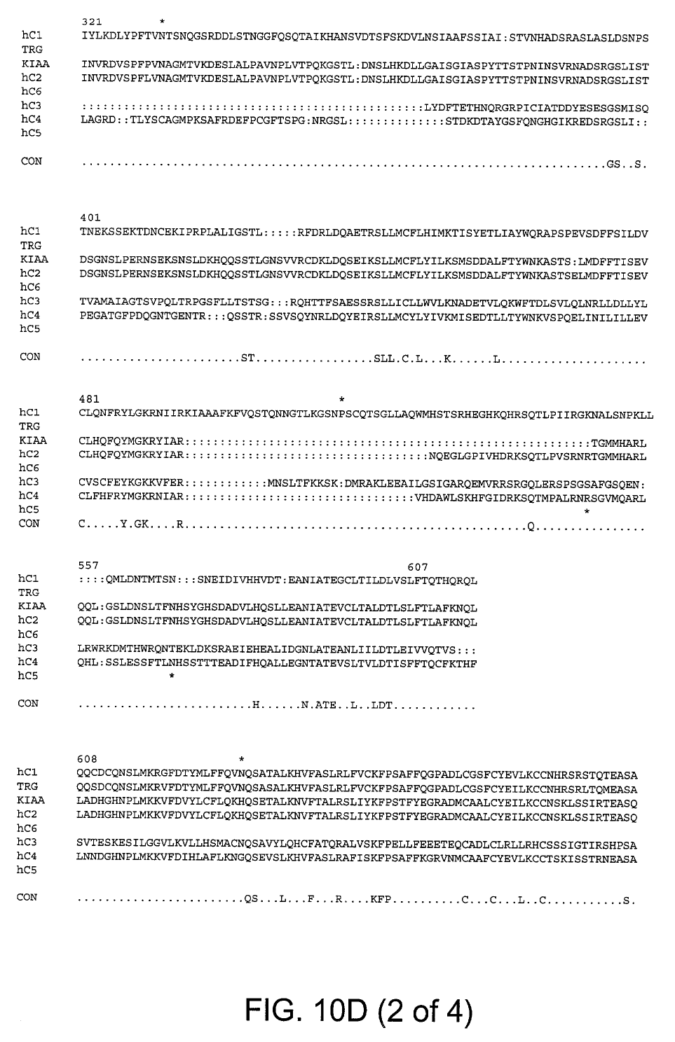

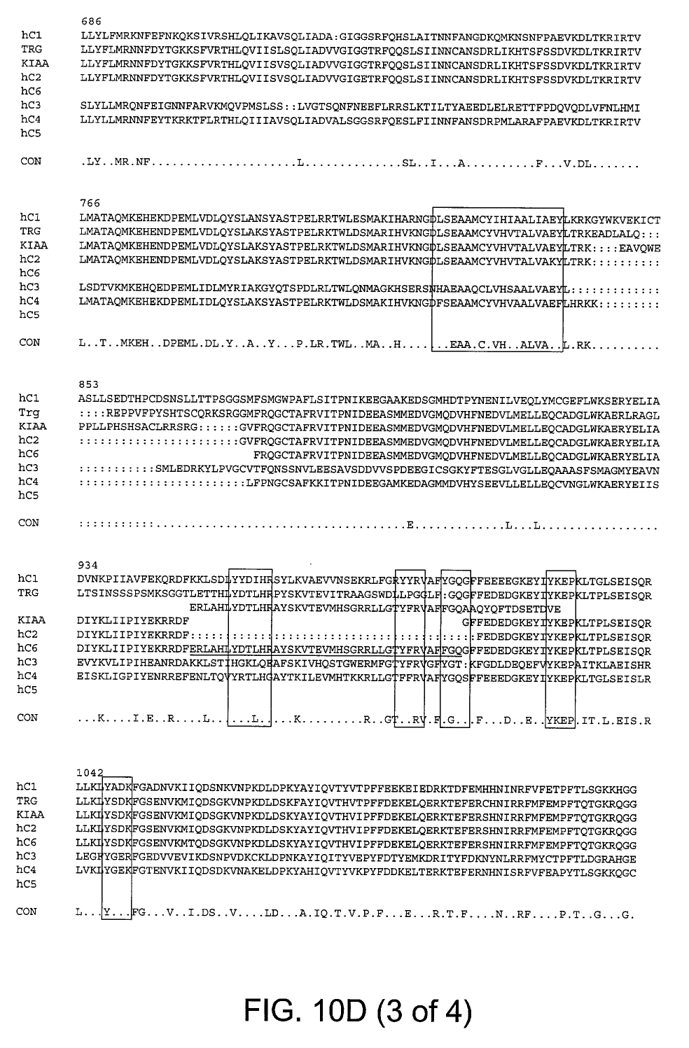

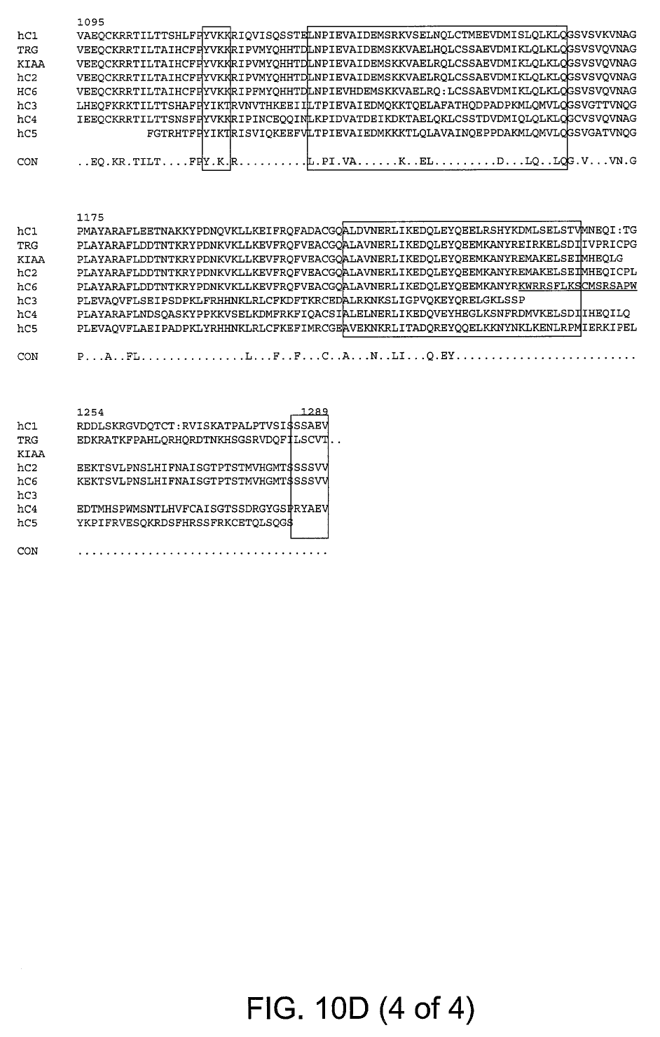

CLASP-4 expression as determined by Northern analysis (FIG. 8) decreases at 4 hours post activation. Minimum expression is detected at 8 hours post activation. A slight increase in CLASP-4-specific RNA is seen at 14 hours post activation. Intensities of CLASP-4-specific signals were quantified by phosphor imager analysis. Rectangles were drawn around the areas of CLASP-4-specific signal and total quantity of signal was determined by the “volume report” mode.

The same Northern-membrane used for the CLASP-4 expression study was hybridized to a human beta-actin probe. The expression profile throughout different post T-cell activation time points is similar to that seen for CLASP-4. This suggests, that both CLASP-4 and beta actin might play a role in the same T-cell activation-specific pathway or in two individual pathways that are functionally interconnected. Alternatively, the decrease of CLASP-4 and of beta actin transcription upon T-cell activation indicates that CLASP-4 and beta actin might form a complex that might be involved in a T-cell activation linked pathway. As determined by ethidium bromide staining, RNA samples were loaded in even quantities onto the corresponding Northern analysis gel. Therefore the changes seen for CLASP-4 and beta-actin expression post T-cell activation cannot be attributed to RNA quantity or quality. No significant change in the intensity of expression signals specific to another probe (CLASP-1) that was hybridized to the same RNA was seen (blot not shown; see diagram), indicating even RNA quality.

### EXAMPLE 4

### Chromosomal location of CLASP-4 and possible disease associations

CLASP-4 cDNA sequences have been mapped to the genomic clone (GI: 5836219) by use of sequence homology bioinformatics tools BLAST.

Clones (GI:5836219) have previously been mapped to the chromosomal location Xq22.3. The literature research reports that the mutations, deletions, rearrangements, disomies and/or breakpoints (in general: chromosomal aberations) in below listed genes make the genes strong candidates for the onset of the listed disease/disorders. Because the CLASP-4 gene is localized in the chromosome location Xq22.3, abnormal CLASP-4 gene regulation or deletion, rearrangement and/or mutations in CLASP-4 locus might be directly or indirectly associated with the onset of the listed diseases. Further, CLASP-4 gene can be used as a genetic probe to detect the abnormality in regions of these below listed genes and as a diagnostic marker for the related disease/disorders.

| CANDIDATE | | RELATED |
|---|---|---|
| GENES | LOCUS | DISEASEDISORDERS |
| COL4A5: | Xq22.3 | Alport synd.: nephritis, neurosensory deafness. |
| Collagen IV, | | AMMECR1: Alport synd. + mental retardation, |
| a5 | | midface hypoplasia |

### EXAMPLE 5

### Tissue and Cell Line Expression of the CLASP-4 gene

Multiple Tissue Northern blots were purchased from Clontech; hybridization procedures were followed according to manufacturer's procedures and recommendations. Human T cell line (Jurkat), human myelomonocyte cells (MV4-11), B cells (9D10), monocytes (THP-1), mouse T cells (3A9), mouse B cells (CH27), human promyelocyte (HL60) and human kidney epithelial cells (293 cell line) were maintained as cultured cell lines. For Multiple Cell Northems, RNA was prepared from cell suspensions using the GIBCO-BRL Trizol system. All steps were performed according to the manufacturer's procedures and recommendations. RNA concentrations were determined by the 260 nm/280 nm light absorption of the RNA solution. 20 μg RNA was ethanol precipitated and resuspended in formamide/formaldehyde buffer and incubated for 15′ at 65° C. to eliminate putative secondary structures. RNA samples were run over night on a 1.1% agarose gel containing 1.5% formaldehyde (both gel and running buffer were 20 mM sodium phosphate, pH 7.5). To visualize RNA after gel migration, approx. 0.5 μg ethidium bromide was added to each sample prior to the run together with RNA loading buffer. RNA in the gel was then visualized by 260 nm wavelength light. After soaking the gel for 15′ in deionized water to reduce the concentration of ethidium bromide in the gel, the RNA was transferred onto Amersham Hybond-N plus membrane by capillary blotting in 20× SSC buffer for 5 hours. Subsequent to blotting, the membrane was washed in 5×SSC for 3′ and RNA was crosslinked to the membrane by UV light (Stratagene Stratalinker).

A CLASP-4-specific DNA fragment (HC4.3) was generated by PCR from a CLASP-4 cDNA clone, using primers129333F2 and 12933R3 (spanning nucleotides 2987-3660 of the cDNA presented in FIG. 1). The HC4.3 probe was prepared using standard labeling kits and desalted using pasteur pipette G-50 Sephadex column in TEN (10 mM Tris-HCl, pH 8.0, 1 mM EDTA, and 100 mM NaCl).

Hybridizations of 32P dCTP labeled DNA probes to the membrane bound RNAs (multiple tissue and multiple cells) were carried out in CLONTECH EXPRESSHYB solution, at 68° C. and for 1-2 hours. Blots were washed 2 times in 2× SSC 0.1% SDS for 10′ each at 50° C. and then twice in 0.2×SSC 0.1% SDS for 10′ each at 50° C., followed by a 5′ wash in 2×SSC at 50° C. Exposure to KODAK BIOMAX MS film was carried out at minus 80° C. using amplifying screens. Typical exposure times were 10 to 36 hours.

A single band is clearly detected migrating at approximately 7.5 kb with strong expression in peripheral blood lymphocytes (PBL). Slight expression is detected in lung, placenta, small intestine, liver, kidney, spleen, thymus, and heart (FIG. 2). A similarly migrating band is detected in Jurkat (T-cell derived), MV4-11 (myelomonocytic), 9D10 (B-cell derived) and 293 (human kidney derived) cell lines.

### EXAMPLE 6

Southern Analysis of CLASP-4

BAC DNA was prepared from E. coli over night cultures using the QIAGEN DNA preparation system. All preps were performed according to the manufacturer's procedures, including the modifications for low copy number DNA constructs. Genomic DNA was prepared from HeLa cells (ATCC #CCL-17) using the methods described by Sambrook, Fritsch and Maniatis (1989); DNA concentrations were determined by the 260 mn light absorption of the DNA solution, and aliquots corresponding to 20 microgram (μg) genomic DNA or 2 μg for BAC DNA were used for restriction enzyme digests with Eco RI or HinD III (genomic DNA) or Eco RI and Pst I (BAC DNA). Digests were carried out in 150 microliter volume for 4 hours at 37° C. Digested DNA was ethanol precipitated and the pellet was resuspended in 20 microliter deionized water prior to migration over a 1.2% agarose gel at 35 V over night. Running buffer was TAE, and the gel contained 0.1 μg ethidium bromide/ml to visualize DNA.

Subsequent to gel separation, DNA was visualized by 260 nm wavelength light. The gel was then washed twice for 20′ in denaturing buffer (0.5M NaCl, 0.4N NaOH) and twice in neutralization buffer (1.5M NaCl, 0.5M TRIS pH 8.0). DNA was transferred from the gel onto AMERSHAM HYBOND N membrane by capillary blotting in 20×SSC for 5 hours. The DNA was crosslinked to the membrane by UV light using a Stratagene Stratalinker.

For probing the Southern, a CLASP-4-specific DNA fragment (HC4. 1) was generated by PCR from a CLASP-4 cDNA , using primers HC4S5′ and HC4AS10. The fragment was labeled by incorporation of radioactive 32P dCTP and desalted using pasteur pipette G-50 Sephadex column in TEN (10 mM Tris-HCl, pH 8.0, 1 mM EDTA, and 100 mM Nacl). Probe HC4.1 is 606 bp long (spanning nucleotides 353-958 of the cDNA sequence shown in FIG. 1 and nucleotides 2319-2924 of the cDNA sequence shown in FIG. 5). Hybridizations of 32P dCTP labeled DNA against DNA immobilized onto the membrane were carried out at 65° C. overnight in modified CHURCH hybridization solution (7% SDS, 0.5M sodiumphosphate, 1 mM EDTA). Membranes were then exposed to KODAK BIOMAX MS film at minus 80° C. Typical exposure times were 12 hours for genomic DNA southern analysis and 3 hours for BAC DNA Southern analysis.

The genomic DNA southern analysis revealed that the hCLASP-4 probe recognizes four fragments (approximately sized at 1.3 kb, 2.8 kb, 4.1 kb and 5.5 kb) on HindIII digested DNA and three fragments (approximately sized at 1.6 kb, 11 kb and 13 kb) on EcoRI digested DNA (FIG. 4).

The present invention is not to be limited in scope by the exemplified embodiments which are intended as illustrations of single aspects of the invention, and any clones, DNA or amino acid sequences which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims. It is also to be understood that all base pair sizes given for nucleotides are approximate and are used for purposes of description.

All publications and patent documents cited above are hereby incorporated by reference in their entirety for all purposes to the same extent as if each were so individually denoted.

CLASP proteins are described in commonly assigned Application Nos. ______; ______; ______; ______ [Attorney Docket Nos. 020054-000311US, 020054-000511US, 020054-000611US] (all filed Dec. 13, 2000), 60/240,508, 60/240,503, 60/240,539, 60/240,543 (all filed Oct. 13, 2000); 09/547,276, 60/196,267, 60/196,527, 60/196,528, 60/196,460 (all filed Apr. 11, 2000); 60/182,296 (filed Feb. 14, 2000), 60/176,195 (filed Jan. 14, 2000), 60/170,453 (filed Dec. 13, 1999), 60/162,498 (filed Oct. 29, 1999), 60/160,860 filed Oct. 21, 1999, 60/129,171 filed Apr. 14, 1999, and in published PCT publications PCT/US00/13161 (WO 00/69896); PCT/US00/13205 (WO 00/69898); PCT/US00/13166 (WO 00/69897); PCT/US00/10158 (WO 00/61747); and PCT/US99/22996 (WO 00/20434). The disclosures of each of the aforementioned applications and publications is expressly incorporated herein by reference in its entirety for all purposes.

What is claimed is:

1. An isolated CLASP-4 polynucleotide, wherein said polynucleotide is
(a) a polynucleotide that has the sequence of SEQ ID NO:1 or
(b) a polynucleotide that hybridizes under stringent hybridization conditions to (a) and encodes a polypeptide having the sequence of SEQ ID NO:2 or an allelic variant or homologue of a polypeptide having the sequence of SEQ ID NO:2; or
(c) a polynucleotide that hybridizes under stringent hybridization conditions to (a) and encodes a polypeptide with at 25 contiguous residues of the polypeptide of SEQ ID NO:2; or
(d) a polynucleotide that hybridizes under stringent hybridization conditions to (a) and has at least 12 contiguous bases identical to or exactly complementary to SEQ ID NO 1.

2. The polynucleotide of claim 1 that encodes a polypeptide having the full-length sequence of SEQ ID NO:2.

3. The isolated polynucleotide of claim 1, comprising the cDNA coding sequence of ATCC accession number PTA-1566, PTA-1572, PTA-2608 or PTA-2610.

4. An isolated CLASP-4 polynucleotide comprising a nucleotide sequence that has at least 90% percent identity to SEQ ID NO: 1.

5. An isolated polypeptide comprising a nucleotide sequence that has at least 90% sequence identity to SEQ ID NO:2 and is immunologically crossreactive with SEQ ID NO:2 or shares a biological function with native CLASP-4.

6. A vector comprising the polynucleotide of claim 1.

7. An expression vector comprising the polynucleotide of claim 1 in which the nucleotide sequence of the polynucleotide is operatively linked with a regulatory sequence that controls expression of the polynucleotide in a host cell.

8. A host cell comprising the polynucleotide of claim 1, or progeny of the cell.

9. A host cell comprising the polynucleotide of claim 1, wherein the nucleotide sequence of the polynucleotide is operatively linked with a regulatory sequence that controls expression of the polynucleotide in a host cell, or progeny of the cell.

10. The host cell of claim 8 which is a eukaryote.

11. The polynucleotide of claim 1 that is an antisense polynucleotide less than about 200 bases in length.

12. An antisense oligonucleotide complementary to a messenger RNA comprising SEQ ID NO:1 and encoding CLASP-4, wherein the oligonucleotide inhibits the expression of CLASP-4.

13. An isolated DNA that encodes a CLASP-4 protein as shown in SEQ ID NO:2.

14. The polynucleotide of claim 1 that is RNA.

15. A method for producing a polypeptide comprising:
(a) culturing the host cell of claim 8 under conditions such that the polypeptide is expressed; and
(b) recovering the polypeptide from the cultured host cell or its cultured medium.

16. An isolated polypeptide encoded by a polynucleotide of claim 1.

17. The polypeptide of claim 16 that has the amino acid sequence of SEQ ID NO:2 or a fragment thereof.

18. The isolated polypeptide of claim 16, wherein the polypeptide is cell-membrane associated.

19. The isolated polypeptide of claim 16, wherein the polypeptide is soluble.

20. The polypeptide of claim 17, wherein the polypeptide is fused with a heterologous polypeptide.

21. An isolated CLASP-4 protein having the sequence as shown in SEQ ID NO:2.

22. A protein comprising the sequence as shown in SEQ. ID. NO:1 and variants thereof that are at least 95% identical to SEQ ID. NO:2 and specifically binds spectrin.

23. An isolated antibody that specifically binds to a polypeptide having the amino acid sequence as shown in SEQ ID NO:2, or a binding fragment thereof.

24. The antibody of claim 23, that is monoclonal.

25. A hybridoma capable of secreting the antibody of claim 24.

26. A method for identifying a compound or agent that binds a CLASP-4 polypeptide comprising:
i) contacting a CLASP-4 polypeptide of claim 17 with the compound or agent under conditions which allow binding of the compound to the CLASP-4 polypeptide to form a complex and
ii) detecting the presence of the complex.

27. A method of detecting a CLASP-4 polypeptide in a sample, comprising:
(a) contacting the sample with an antibody or binding fragment of claim 24 and (b) determining whether a complex has been formed between the antibody and with CLASP-4 polypeptide.

28. A method of detecting a CLASP-4 polypeptide in a sample, comprising:
(a) contacting the sample with a polynucleotide of claim 1 or a polynucleotide that comprises a sequence of at least 12 nucleotides and is complementary to a contiguous sequence of the polynucleotide of section (a) of claim 1, and (b) determining whether a hybridization complex has been formed.

29. A method of detecting a CLASP-4 nucleotide in a sample, comprising:
(a) using a polynucleotide that comprises a sequence of at least 12 nucleotides and is complementary to a contiguous sequence of the polynucleotide of section (a) of claim 1, in an amplification process; and
(b) determining whether a specific amplification product has been formed.

30. A pharmaceutical composition comprising a polynucleotide of claim 1, a polypeptide of claim 16, or an antibody of claim 23 and a pharmaceutically acceptable carrier.

31. A method of inhibiting an immune response in a cell comprising:
(a) interfering with the expression of a CLASP-4 gene in the cell;
(b) interfering with the ability of a CLASP-4 protein to bind to another cell;
(c) interfering with the ability of a CLASP-4 protein to bind to another protein.

32. The method of claim 31, wherein the cell is a T cell or a B cell.

33. The method of claim 31 comprising contacting the cell with an effective amount of a polypeptide which comprises the amino acid sequence of SEQ ID NO:2 or a fragment thereof.

34. A method of inhibiting an immune response in a subject, comprising administering to the subject a therapeutically effective amount of an antibody which specifically binds a polypeptide having the sequence of SEQ ID NO:2.

35. A method of preventing or treating a CLASP-4-mediated disease comprising administering to a subject in need thereof a therapeutically effective amount of a pharmaceutical composition of claim 30.

36. The method claim 35, wherein the CLASP-4-mediated disease is an autoimmune disease.

37. A method of treating an autoimmune disease in a subject caused or exacerbated by increased activity of T_{H}1 cells consisting of administering a therapeutically effective amount of a pharmaceutical composition of claim 30 to the subject.
